(19) United States
(12) Patent Application Publication
(10) Pub. No.: US 2023/0416367 A1
(43) Pub. Date: Dec. 28, 2023

(54) COMBINATION THERAPY OF ANTI-PD-1 ACTIVE AGENT, ANTI-TIM-3 ACTIVE AGENT, AND ANTI-LAG-3 ACTIVE AGENT FOR TREATING CANCER

(71) Applicant: Incyte Corporation, Wilmington, DE (US)

(72) Inventors: Paul E. Hoyle, Avondale, PA (US); Zhiwan Dong, Philadelphia, PA (US); Thomas Condamine, Wynnewood, PA (US); Nithya Srinivasan, Weehawken, NJ (US); John E. Janik, North Augusta, SC (US); Kevin N. Heller, Potomac, MD (US)

(21) Appl. No.: 18/330,141

(22) Filed: Jun. 6, 2023

Related U.S. Application Data
(60) Provisional application No. 63/349,876, filed on Jun. 7, 2022.
(60) Provisional application No. 63/405,243, filed on Sep. 9, 2022.

(51) Int. Cl. C07K 16/28; A61P 35/00; A61K 9/00; A61P 35/04

(52) U.S. Cl. CPC C07K 16/2818; C07K 16/2803; A61P 35/00; A61K 9/0019; A61P 35/04; A61K 2039/507

(57) ABSTRACT

The present disclosure provides a combination therapy which comprises: (i) an active agent that binds PD-1 (e.g., an anti-PD-1 antibody), (ii) an active agent that binds TIM-3 (e.g., an anti-TIM-3 antibody), and/or (iii) an active agent that binds LAG-3 (e.g., an anti-LAG-3 antibody). The present disclosure provides pharmaceutical compositions thereof, uses thereof, and methods of treatment which include administering the combination therapy to a subject, including methods of treating cancer.

### RELATED APPLICATIONS

This application claims priority to U.S. Provisional Patent Application No. 63/349,876, filed Jun. 7, 2022, and U.S. Provisional Patent Application No. 63/405,243, filed Sep. 9, 2022. The entire contents of these applications are incorporated herein by reference.

### SEQUENCE LISTING

This application contains a Sequence Listing which has been submitted electronically in XML format and is hereby incorporated by reference in its entirety. The XML copy, created on Jun. 6, 2023, is named 12092-0051-00000_SL.XML and is 57,781 bytes in size.

### FIELD OF THE DISCLOSURE

The present disclosure provides a combination therapy which comprises: (i) an active agent that binds PD-1 (e.g., an anti-PD-1 antibody), (ii) an active agent that binds TIM-3 (e.g., an anti-TIM-3 antibody), and/or (iii) an active agent that binds LAG-3 (e.g., an anti-LAG-3 antibody). The present disclosure provides pharmaceutical compositions thereof, uses thereof, kits thereof, and methods of treatment which include administering the combination therapy to a subject, including methods of treating cancer.

### DISCLOSURE

Significant focus has been placed on understanding the activity of the immune system within the tumor microenvironment and identification of mechanisms of tumor resistance to immune surveillance. Loss of CD8+ T-cell effector function has been one such proposed mechanism. This loss of function, or T-cell exhaustion, has been described to be due in part to the accumulation and increased diversity of inhibitory receptors, or checkpoints, on the cell surface of CD8+ T-cells (Wherry et al., Nat Immunol (2011); 12:492-499).

While antibodies directed towards the PD-1/PDL-1 axis have improved the treatment of cancer, the majority of clinical patients still either fail to respond or lose response to these drugs. Patients who relapse or are resistant to therapy with PD-1 inhibitors represent a high unmet need in oncology. Early clinical evidence of the efficacy of combination therapies in patients who have failed anti-PDL-1 therapy have been evolving. Blockade of the PDL-1 axis in combination with blockade of either LAG-3 or TIM-3 has resulted in only modest responses in patients who have failed anti-PD-1 therapy alone (Ascierto et al., Ann Oncol (2017); 28(suppl 5):v605-v649; Davar et al., Society for Immunotherapy of Cancer (2018); Nov. 7-11, 2018; Washington, DC. Abstract O21).

Thus, it remains challenging to intervene and disrupt the process of T-cell exhaustion using combinatorial blockades of checkpoints along the hierarchy of exhaustion. Potentially, multiple checkpoint inhibitors are required in order to restore anti-tumor T-cell effector function in situ. A need therefore exists for improved combination therapies for the treatment of PD-1-related diseases, such as cancer.

### SUMMARY

The details of various embodiments of the present disclosure are set forth in the description below. In certain embodiments, the present disclosure provides a combination therapy comprising at least two (e.g., three) of the following: a therapeutically effective amount of an anti-PD-1 active agent, a therapeutically effective amount of an anti-TIM-3 active agent, and a therapeutically effective amount of an anti-LAG-3 active agent. In certain embodiments, the combination therapy comprises at least two (e.g., three) of the following: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; (ii) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent; and (iii) from about 300 mg to about 800 mg, in some embodiments about 350 mg to about 750 mg, and in some embodiments about 350 mg, about 450 mg or about 750 mg of an anti-LAG-3 active agent. In certain embodiments, the combination therapy comprises: (a) the anti-PD-1 active agent and the anti-TIM-3 active agent; (b) the anti-PD-1 active agent and the anti-LAG-3 active agent; (c) the anti-TIM-3 active agent and the anti-LAG-3 active agent; or (d) the anti-PD-1 active agent, the anti-TIM-3 active agent, and the anti-LAG-3 active agent. In certain embodiments, the combination therapy comprises the anti-PD-1 active agent and the anti-TIM-3 active agent. In certain embodiments, the combination therapy comprises the anti-PD-1 active agent and the anti-LAG-3 active agent. In certain embodiments, the combination therapy comprises the anti-TIM-3 active agent and the anti-LAG-3 active agent. In certain embodiments, the combination therapy comprises the anti-PD-1 active agent, the anti-TIM-3 active agent, and the anti-LAG-3 active agent.

In certain embodiments, the combination therapy is for use in the treatment of cancer in a human subject in need thereof. In certain embodiments, the combination therapy is for use in a method for treating cancer in a human subject in need thereof. In certain embodiments, the combination therapy is for use in the manufacturing/production of a medicament for use in treating cancer in a human subject in need thereof.

In certain embodiments, the present disclosure provides an anti-PD-1 active agent for use in treating cancer in a human subject in need thereof, wherein the anti-PD-1 active agent is administered at a therapeutically effective amount, concurrently or sequentially with at least one (e.g., both) of the following: a therapeutically effective amount of an anti-TIM-3 active agent and a therapeutically effective amount of an anti-LAG-3 agent. In certain embodiments, the anti-PD-1 active agent is administered at a dose from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg; and is administered concurrently or sequentially with at least one (e.g., both) of the following: (i) about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent, and (ii) about 300 mg to about 800 mg, in some embodiments about 350 mg to about 750 mg, and in some embodiments about 350 mg, about 450 mg or about 750 mg of an anti-LAG-3 active agent. In certain embodiments, the anti-PD-1 active agent is administered at a dose from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, once every three weeks or once every four weeks, and is administered concurrently or sequentially with at least one (e.g., both) of the following: (i) about 300 mg to about 1000 mg, in some embodiments about 400 to about 1000 mg, in some embodiments about 350 mg to about 500 mg, and in some embodiments about 400 mg, about 500 mg or about 1000 mg of an anti-TIM-3 active agent which is administered once every two weeks, once every three weeks, or once every four weeks, and (ii) about 300 mg to about 800 mg, in some embodiments about 350 mg to about 750 mg, and in some embodiments about 350 mg, about 450 or about 750 mg of an anti-LAG-3 active agent which is administered once every two weeks, once every three weeks, or once every four weeks. In certain embodiments, the anti-PD-1 active agent is administered concurrently or sequentially with (a) the anti-TIM-3 active agent, (b) the anti-LAG-3 active agent, or (c) both the anti-TIM-3 active agent and the anti-LAG-3 active agent. In certain embodiments, the anti-PD-1 active agent is administered concurrently or sequentially with the anti-TIM-3 active agent. In certain embodiments, the anti-PD-1 active agent is administered concurrently or sequentially with the anti-LAG-3 active agent. In certain embodiments, the anti-PD-1 active agent is administered concurrently or sequentially with both the anti-TIM-3 active agent and the anti-LAG-3 active agent.

In certain embodiments, the present disclosure provides an anti-PD-1 active agent, an anti-TIM-3 active agent, and an anti-LAG-3 agent for use in treating cancer in a human subject in need thereof, wherein the anti-PD-1 active agent is administered at a therapeutically effective amount, the anti-TIM-3 active agent is administered at a therapeutically effective amount, and the anti-LAG-3 active agent is administered at a therapeutically effective amount. In certain embodiments: (i) the anti-PD-1 active agent is administered at a dose from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg; (ii) the anti-TIM-3 active agent is administered at a dose from about 300 mg to about 1000 mg, in some embodiments about 400 mg to about 1000 mg, in some embodiments about 350 mg to about 500 mg, and in some embodiments about 400 mg, about 500 mg or about 1000 mg; and (iii) the anti-LAG-3 active agent is administered at a dose from about 300 mg to about 800 mg, in some embodiments about 350 mg to about 750 mg, and in some embodiments about 350 mg, about 450 mg or about 750 mg. In certain embodiments: (i) the anti-PD-1 active agent is administered at a dose from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, once every three to five weeks, and in some embodiments once every three weeks or once every four weeks; (ii) the anti-TIM-3 active agent is administered at a dose from about 300 mg to about 1000 mg, in some embodiments about 400 mg to about 1000 mg, in some embodiments about 350 mg to about 500 mg, and in some embodiments about 400 mg, about 500 mg or about 1000 mg, once every one to four weeks, and in some embodiments once every two weeks, once every three weeks, or once every four weeks; and (iii) the anti-LAG-3 active agent is administered at a dose from about 300 mg to about 800 mg, in some embodiments about 350 mg to about 750 mg, and in some embodiments about 350 mg, about 450 mg or about 750 mg, once every one to four weeks, and in some embodiments once every two weeks, once every three weeks, or once every four weeks. In certain embodiments, the present disclosure provides the use of the anti-PD-1 active agent, the anti-TIM-3 active agent, and the anti-LAG-3 active agent, in the treatment of cancer in a human subject in need thereof. In certain embodiments, the present disclosure provides the use of the anti-PD-1 active agent, the anti-TIM-3 active agent, and the anti-LAG-3 active agent, in the manufacturing/production of a medicament for use in treating cancer in a human subject in need thereof.

In certain embodiments, the present disclosure provides a method for treating cancer in a human subject in need thereof, the method comprising administering to the subject at least two (e.g., three) of the following: (i) a therapeutically effective amount of an anti-PD-1 active agent; (ii) therapeutically effective amount of an anti-TIM-3 active; and (iii) therapeutically effective amount of an anti-LAG-3 active agent. In certain embodiments, the method comprises administering to the subject at least two (e.g., three) of the following: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; (ii) from about 300 to about 1000 mg, in some embodiments about 400 to about 1000, in some embodiments about 350 to about 500 mg, and in some embodiments about 400 mg, about 500 mg or about 1000 mg, of an anti-TIM-3 active agent; and (iii) from about 300 mg to about 800 mg, in some embodiments about 350 mg to about 750 mg, and in some embodiments about 350 mg, about 450 mg or about 750 mg, of an anti-LAG-3 active agent. In certain embodiments, the method comprises administering to the subject at least two (e.g., three) of the following: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent once every three to five weeks, and in some embodiments one every three weeks or once every four weeks; (ii) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent once every one to four weeks, and in some embodiments once every two weeks, once every three weeks, or once every four weeks; and (iii) from about 300 mg to about 800 mg, in some embodiments about 350 mg to about 750 mg, and in some embodiments about 350 mg, about 450 mg or about 750 mg of an anti-LAG-3 active agent once every one to four weeks, and in some embodiments once every two weeks, once every three weeks, or once every four weeks. In certain embodiments, the method comprises: (a) administering to the subject the anti-PD-1 active agent and the anti-TIM-3 active agent; (b) administering to the subject the anti-PD-1 active agent and the anti-LAG-3 active agent; (c) administering to the subject the anti-TIM-3 active agent and the anti-LAG-3 active agent; or (d) administering to the subject the anti-PD-1 active agent, the anti-TIM-3 active agent, and the anti-LAG-3 active agent. In certain embodiments, the method comprises administering to the subject the anti-PD-1 active agent and the anti-TIM-3 active agent. In certain embodiments, the method comprises administering to the subject the anti-PD-1 active agent and the anti-LAG-3 active agent. In certain embodiments, the method comprises administering to the subject the anti-TIM-3 active agent and the anti-LAG-3 active agent. In certain embodiments, the method comprises administering to the subject the anti-PD-1 active agent, the anti-TIM-3 active agent, and the anti-LAG-3 active agent.

In certain embodiments, the present disclosure provides a kit for treating a cancer in a human subject in need thereof, the kit comprising at least two (e.g., three) of the following: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; (ii) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent; and (iii) from about 300 mg to about 800 mg, in some embodiments about 350 mg to about 750 mg, and in some embodiments about 350 mg, about 450 mg or about 750 mg, of an anti-LAG-3 active agent. In certain embodiments, the kit further comprises instructions to administer the anti-PD-1 active agent (e.g., once every three weeks or once every four weeks), to administer the anti-TIM-3 active agent (e.g., once every two weeks, once every three weeks, or once every four weeks), and/or to administer the anti-LAG-3 active agent (e.g., once every two weeks, once every three weeks, or once every four weeks).

In certain embodiments, the present methods, combination therapies, uses, or kits of the present disclosure comprise an anti-PD-1 active agent. In certain embodiments, the anti-PD-1 active agent comprises an anti-PD-1 antibody, or PD-1 binding-fragment thereof. In certain embodiments, the anti-PD-1 antibody is retifanlimab (also known as MGA012 and INCMGA00012; CAS Reg No. 2079108-44-2). In certain embodiments, the anti-PD-1 active agent is administered intravenously. In certain embodiments, the anti-PD-1 active agent is administered intravenously once every three weeks or once every four weeks. In certain embodiments, the anti-PD-1 active agent is administered intravenously once every three weeks. In certain embodiments, the anti-PD-1 active agent is administered intravenously once every four weeks.

In certain embodiments, the anti-PD-1 active agent comprises an anti-PD-1 antibody, or PD-1 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain. In certain embodiments, the CDRH1 Domain has the amino acid sequence SYWMN (SEQ ID NO: 1). In certain embodiments, the CDRH2 Domain has the amino acid sequence VIIIPSDSETWLDQKFK (SEQ ID NO: 2). In certain embodiments, the CDRH3 Domain has the amino acid sequence EHYGTSPFAY (SEQ ID NO: 3). In certain embodiments, the CDRH1 Domain has the amino acid sequence SYWMN (SEQ ID NO: 1); the CDRH2 Domain has the amino acid sequence VIHPSDSETWLDQKFK (SEQ ID NO: 2); and the CDRH3 Domain has the amino acid sequence EHYGTSPFAY (SEQ ID NO: 3). In certain embodiments, the CDRL1 Domain has the amino acid sequence RASESVDNYGMSFMNW (SEQ ID NO: 4). In certain embodiments, the CDRL2 Domain has the amino acid sequence AASNQGS (SEQ ID NO: 5). In certain embodiments, the CDRL3 Domain has the amino acid sequence QQSKEVPYT (SEQ ID NO: 6). In certain embodiments, the CDRL1 Domain has the amino acid sequence RASESVDNYGMSFMNW (SEQ ID NO: 4); the CDRL2 Domain has the amino acid sequence AASNQGS (SEQ ID NO: 5); and the CDRL3 Domain has the amino acid sequence QQSKEVPYT (SEQ ID NO: 6). In certain embodiments, the CDRH1 Domain has the amino acid sequence SYWMN (SEQ ID NO: 1); the CDRH2 Domain has the amino acid sequence VIIIPSDSETWLDQKFK (SEQ ID NO: 2); the CDRH3 Domain has the amino acid sequence EHYGTSPFAY (SEQ ID NO: 3); the CDRL1 Domain has the amino acid sequence RASESVDNYGMSFMNW (SEQ ID NO: 4); the CDRL2 Domain has the amino acid sequence AASNQGS (SEQ ID NO: 5); and the CDRL3 Domain has the amino acid sequence QQSKEVPYT (SEQ ID NO: 6).

In certain embodiments, the Heavy Chain Variable Domain of the anti-PD-1 antibody or fragment thereof comprises an amino acid sequence which is at least 75%, 80%, 85%, 90%, 95%, 99%, or 100% identical to: QVQLVQSGAEVKKPGASVKVSCKASGYSFTSYWMNWVRQAPGQGLEWIGVIIIPSDSE TWLDQKFKDRVTITVDKSTSTAYMELSSLRSEDTAVYYCAREHYGTSPFAYWGQGTLV TVSS (SEQ ID NO: 7). In certain embodiments, the Light Chain Variable Domain of the anti-PD-1 antibody or fragment thereof comprises an amino acid sequence which is at least 75%, 80%, 85%, 90%, 95%, 99%, or 100% identical to: EIVLTQSPATL SLSPGERATLSCRASESVDNYGMSFMNWFQQKPGQPPKLLIHAASNQGS GVPSRFSGSGSGTDFTLTISSLEPEDFAVYFCQQSKEVPYTFGGGTKVEIK (SEQ ID NO: 8). In certain embodiments, the Heavy Chain Variable Domain of the anti-PD-1 antibody or fragment thereof comprises an amino acid sequence which is at least 75%, 80%, 85%, 90%, 95%, 99%, or 100% identical to: QVQLVQSGAEVKKPGASVKVSCKASGYSFTSYWMNWVRQAPGQGLEWIGVIIIPSDSE TWLDQKFKDRVTITVDKSTSTAYMELSSLRSEDTAVYYCAREHYGTSPFAYWGQGTLV TVSS (SEQ ID NO: 7); and the Light Chain Variable Domain of the anti-PD-1 antibody or fragment thereof comprises an amino acid sequence which is at least 75%, 80%, 85%, 90%, 95%, 99%, or 100% identical to:

(SEQ ID NO: 8)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIK.

In certain embodiments, the anti-PD-1 antibody or fragment thereof comprises a Heavy Chain (HC) sequence which is at least 75%, 80%, 85%, 90%, 95%, 99%, or 100% identical to: QVQLVQSGAEVKKPGASVKVSCKASGYSFTSYWMNWVRQAPGQGLEWIGVIIIPSDSE TWLDQKFKDRVTITVDKSTSTAYMELSSLRSEDTAVYYCAREHYGTSPFAYWGQGTLV TVSSASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRVESKYGPPCPPCPAPEFLG GPSVFLFPPKPKDTLMISRTPEVTCVVVDVSQEDPEVQFNWYVDGVEVHNAKTKPREEQ FNSTYRVVSVLTVLHQDWLNGKEYKCKVSNKGLPSSIEKTISKAKGQPREPQVYTLPPS QEEMTKNQVSLTCLVKGFYPSDIAVEWESNGQPENNYKTTPPVLDSDGSFFLYSRLTVD KSRWQEGNVFSCSVMHEALHNHYTQKSLSLSLG (SEQ ID NO: 9). In certain embodiments, the anti-PD-1 antibody or fragment thereof comprises a Light Chain (LC) sequence which is at least 75%, 80%, 85%, 90%, 95%, 99%, or 100% identical to: EIVLTQSPATLSLSPGERATLSCRASESVDNYGMSFMNWFQQKPGQPPKLLIHAASNQGS GVPSRFSGSGSGTDFTLTISSLEPEDFAVYFCQQSKEVPYTFGGGTKVEIKRTVAAPSVFIF PPSDEQLKSGTASVVCLLNNFYPREAKVQWKVDNALQSGNSQESVTEQDSKDSTYSLSS TLTLSKADYEKHKVYACEVTHQGLSSPVTKSFNRGEC (SEQ ID NO: 10). In certain embodiments, the anti-PD-1 antibody or fragment thereof comprises a Heavy Chain (HC) sequence which is at least 75%, 80%, 85%, 90%, 95%, 99%, or 100% identical to: QVQLVQSGAEVKKPGASVKVSCKASGYSFTSYWMNWVRQAPGQGLEWIGVIIIPSDSE TWLDQKFKDRVTITVDKSTSTAYMELSSLRSEDTAVYYCAREHYGTSPFAYWGQGTLV TVSSASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRVESKYGPPCPPCPAPEFLG GPSVFLFPPKPKDTLMISRTPEVTCVVVDVSQEDPEVQFNWYVDGVEVHNAKTKPREEQ FNSTYRVVSVLTVLHQDWLNGKEYKCKVSNKGLPSSIEKTISKAKGQPREPQVYTLPPS QEEMTKNQVSLTCLVKGFYPSDIAVEWESNGQPENNYKTTPPVLDSDGSFFLYSRLTVD KSRWQEGNVFSCSVMHEALHNHYTQKSLSLSLG (SEQ ID NO: 9); and the anti-PD-1 antibody or fragment thereof comprises a Light Chain (LC) sequence which is at least 75%, 80%, 85%, 90%, 95%, 99%, or 100% identical to:

(SEQ ID NO: 10)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In certain embodiments, the anti-PD-1 antibody or fragment thereof comprises an Fc Region that is of the IgG1, IgG2, IgG3, or IgG4 isotype. In certain embodiments, the anti-PD-1 antibody or fragment thereof comprises an Fe Region that is of the IgG4 isotype. In certain embodiments, the antibody comprises a Hinge Domain of the IgG4 isotype that comprises a stabilizing mutation. In certain embodiments, the anti-PD-1 antibody or fragment thereof comprises a variant Fe Region that comprises: (A) one or more amino acid modifications that reduce the affinity of the variant Fc Region for an FcγR, wherein the one or more modifications that reduce the affinity of the variant Fc Region for an FcγR comprise the substitution of L234A, L235A, or L234A+L235A, wherein the numbering is that of the EU index as in Kabat; and/or (B) one or more amino acid modifications that enhance the serum half-life of the variant Fc Region, wherein the one or more modifications that enhance the serum half-life of the variant Fc Region comprise the substitution of M252Y, M252Y+S254T, M252Y+T256E, M252Y+S254T+T256E, or K288D+H435K, wherein the numbering is that of the EU index as in Kabat.

In certain embodiments, the present methods, combination therapies, uses, or kits of the present disclosure comprise an anti-TIM-3 active agent. In certain embodiments, the anti-TIM-3 active agent comprises an anti-TIM-3 antibody, or TIM-3 binding-fragment thereof. In certain embodiments, the anti-TIM-3 antibody is Antibody A, as described herein. In certain embodiments, the anti-TIM-3 active agent is administered intravenously. In certain embodiments, the anti-TIM-3 active agent is administered intravenously once every two weeks, once every three weeks, or once every four weeks. In certain embodiments, the anti-TIM-3 active agent is administered intravenously once every two weeks. In certain embodiments, the anti-TIM-3 active agent is administered intravenously once every three weeks. In certain embodiments, the anti-TIM-3 active agent is administered intravenously once every four weeks.

In certain embodiments, the anti-TIM-3 active agent comprises an anti-TIM-3 antibody, or TIM-3 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain. In certain embodiments, the CDRH1 Domain has the amino acid sequence RQNAWS (SEQ ID NO: 11). In certain embodiments, the CDRH2 Domain has the amino acid sequence WVSAISGSGGSTY (SEQ ID NO: 12). In certain embodiments, the CDRH3 Domain has the amino acid sequence AKGGDYGGNYFD (SEQ ID NO: 13). In certain embodiments, the CDRH1 Domain has the amino acid sequence RQNAWS (SEQ ID NO: 11); the CDRH2 Domain has the amino acid sequence WVSAISGSGGSTY (SEQ ID NO: 12); and the CDRH3 Domain has the amino acid sequence AKGGDYGGNYFD (SEQ ID NO: 13). In certain embodiments, the CDRL1 Domain has the amino acid sequence RASQSVSSYLA (SEQ ID NO: 14). In certain embodiments, the CDRL2 Domain has the amino acid sequence DASNRAT (SEQ ID NO: 15). In certain embodiments, the CDRL3 Domain has the amino acid sequence QQYGSSPLT (SEQ ID NO: 16). In certain embodiments, the CDRL1 Domain has the amino acid sequence RASQSVSSYLA (SEQ ID NO: 14); the CDRL2 Domain has the amino acid sequence DASNRAT (SEQ ID NO: 15); and the CDRL3 Domain has the amino acid sequence QQYGSSPLT (SEQ ID NO: 16). In certain embodiments, the CDRH1 Domain has the amino acid sequence RQNAWS (SEQ ID NO: 11); the CDRH2 Domain has the amino acid sequence WVSAISGSGGSTY (SEQ ID NO: 12); the CDRH3 Domain has the amino acid sequence AKGGDYGGNYFD (SEQ ID NO: 13); the CDRL1 Domain has the amino acid sequence RASQSVSSYLA (SEQ ID NO: 14); the CDRL2 Domain has the amino acid sequence DASNRAT (SEQ ID NO: 15); and the CDRL3 Domain has the amino acid sequence QQYGSSPLT (SEQ ID NO: 16).

In certain embodiments, the Heavy Chain Variable Domain of the anti-TIM-3 antibody or fragment thereof comprises an amino acid sequence which is at least 75%, 80%, 85%, 90%, 95%, 99%, or 100% identical to: EVQLVESGGGLVQPGGSLRLSCAASGFTFRQNAWSWVRRAPGKGLEWVSAISGSGGST YYADSVKGRFTISRDNSKNTLYLQMNSLRAEDTAVYYCAKGGDYGGNYFDYWGQGTL VTVSS (SEQ ID NO: 17). In certain embodiments, the Light Chain Variable Domain of the anti-TIM-3 antibody or fragment thereof comprises an amino acid sequence which is at least 75%, 80%, 85%, 90%, 95%, 99%, or 100% identical to: EIVLTQSPATLSLSPGERATLSCRASQSVSSYLAWYQQKPGQAPRLLIYDASNRATGIPAS FSGSGSGTDFTLTISRLEPEDFAVYYCQQYGSSPLTFGGGTKVEIK (SEQ ID NO: 18). In certain embodiments, the Heavy Chain Variable Domain of the anti-TIM-3 antibody or fragment thereof comprises an amino acid sequence which is at least 75%, 80%, 85%, 90%, 95%, 99%, or 100% identical to: EVQLVESGGGLVQPGGSLRLSCAASGFTFRQNAWSWVRRAPGKGLEWVSAISGSGGST YYADSVKGRFTISRDNSKNTLYLQMNSLRAEDTAVYYCAKGGDYGGNYFDYWGQGTL VTVSS (SEQ ID NO: 17); and the Light Chain Variable Domain of the anti-TIM-3 antibody or fragment thereof comprises an amino acid sequence which is at least 75%, 80%, 85%, 90%, 95%, 99%, or 100% identical to:

(SEQ ID NO: 18)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIK.

In certain embodiments, the anti-TIM-3 antibody or fragment thereof comprises a Heavy Chain (HC) sequence which is at least 75%, 80%, 85%, 90%, 95%, 99%, or 100% identical to: EVQLVESGGGLVQPGGSLRLSCAASGFTFRQNAWSWVRRAPGKGLEWVSAISGSGGST YYADSVKGRFTISRDNSKNTLYLQMNSLRAEDTAVYYCAKGGDYGGNYFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKRVEPKSCDKTHTCPPCPAP ELLGGPSVFLFPPKPKDTLMISRTPEVTCVVVDVSSHEDPEVKFNWYVDGVEVHNAKTKP REEQYASTYRVVSVLTVLHQDWLNGKEYKCKVSNKALPAPIEKTISKAKGQPREPQVY TLPPSREEMTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 19). In certain embodiments, the anti-TIM-3 antibody or fragment thereof comprises a Light Chain (LC) sequence which is at least 75%, 80%, 85%, 90%, 95%, 99%, or 100% identical to: EIVLTQSPATLSLSPGERATLSCRASQSVSSYLAWYQQKPGQAPRLLIYDASNRATGIPAS FSGSGSGTDFTLTISRLEPEDFAVYYCQQYGSSPLTFGGGTKVEIKRTVAAPSVFIFPPSDE QLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 20). In certain embodiments, the anti-TIM-3 antibody or fragment thereof comprises a Heavy Chain (HC) sequence which is at least 75%, 80%, 85%, 90%, 95%, 99%, or 100% identical to: EVQLVESGGGLVQPGGSLRLSCAASGFTFRQNAWSWVRRAPGKGLEWVSAISGSGGST YYADSVKGRFTISRDNSKNTLYLQMNSLRAEDTAVYYCAKGGDYGGNYFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKRVEPKSCDKTHTCPPCPAP ELLGGPSVFLFPPKPKDTLMISRTPEVTCVVVDVSSHEDPEVKFNWYVDGVEVHNAKTKP REEQYASTYRVVSVLTVLHQDWLNGKEYKCKVSNKALPAPIEKTISKAKGQPREPQVY TLPPSREEMTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 19); and the anti-TIM-3 antibody or fragment thereof comprises a Light Chain (LC) sequence which is at least 75%, 80%, 85%, 90%, 95%, 99%, or 100% identical to:

(SEQ ID NO: 20)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In certain embodiments, the anti-TIM-3 antibody or fragment thereof comprises an Fc Region that is of the IgG1, IgG2, IgG3, IgG4, IgA1, or IgA2 isotype. In certain embodiments, the anti-TIM-3 antibody or fragment thereof comprises an Fc Region that is of a human IgG1 isotype, wherein the amino acid sequence of the IgG1 heavy chain constant region comprises: an N297A mutation, numbered according to the EU numbering system; or an N297Q mutation, numbered according to the EU numbering system. In certain embodiments, the anti-TIM-3 antibody or fragment thereof comprises an Fc Region of a human IgG4 isotype, wherein the amino acid sequence of the IgG4 heavy chain constant region comprises: an S228P mutation, numbered according to the EU numbering system; or an N297Q mutation, numbered according to the EU numbering system. In certain embodiments, the anti-TIM-3 antibody or fragment thereof is antagonistic to human TIM-3. In certain embodiments, the anti-TIM-3 antibody or fragment thereof deactivates, reduces, or inhibits an activity of human TIM-3. In certain embodiments, the anti-TIM-3 antibody or fragment thereof inhibits binding of human TIM-3 to phosphatidylserine.

In certain embodiments, the present methods, combination therapies, uses, or kits of the present disclosure comprise an anti-LAG-3 active agent. In certain embodiments, the anti-LAG-3 active agent comprises an anti-LAG-3 antibody, or LAG-3 binding-fragment thereof. In certain embodiments, the anti-LAG-3 antibody is Antibody B (as described herein). In certain embodiments, the anti-LAG-3 active agent is administered intravenously. In certain embodiments, the anti-LAG-3 active agent is administered intravenously once every two weeks, once every three weeks, or once every four weeks. In certain embodiments, the anti-LAG-3 active agent is administered intravenously once every two weeks. In certain embodiments, the anti-LAG-3 active agent is administered intravenously once every three weeks. In certain embodiments, the anti-LAG-3 active agent is administered intravenously once every four weeks.

In certain embodiments, the anti-LAG-3 active agent comprises an anti-LAG-3 antibody, or LAG-3 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain. In certain embodiments, the CDRH1 Domain has the amino acid sequence DTYIH (SEQ ID NO: 21). In certain embodiments, the CDRH2 Domain has the amino acid sequence EIDPANDNTKYDPKFQG (SEQ ID NO: 22). In certain embodiments, the CDRH3 Domain has the amino acid sequence YYYKYDVGGFDY (SEQ ID NO: 23). In certain embodiments, the CDRH1 Domain has the amino acid sequence DTYIH (SEQ ID NO: 21); the CDRH2 Domain has the amino acid sequence EIDPANDNTKYDPKFQG (SEQ ID NO: 22); and the CDRH3 Domain has the amino acid sequence YYYKYDVGGFDY (SEQ ID NO: 23). In certain embodiments, the CDRL1 Domain has the amino acid sequence SVSSSISSSNLH (SEQ ID NO: 24). In certain embodiments, the CDRL2 Domain has the amino acid sequence GTSNLAS (SEQ ID NO: 25). In certain embodiments, the CDRL3 Domain has the amino acid sequence QQWSSYPFT (SEQ ID NO: 26). In certain embodiments, the CDRL1 Domain has the amino acid sequence SVSSSISSSNLH (SEQ ID NO: 24); the CDRL2 Domain has the amino acid sequence GTSNLAS (SEQ ID NO: 25); and the CDRL3 Domain has the amino acid sequence QQWSSYPFT (SEQ ID NO: 26). In certain embodiments, the CDRH1 Domain has the amino acid sequence DTYIH (SEQ ID NO: 21); the CDRH2 Domain has the amino acid sequence EIDPANDNTKYDPKFQG (SEQ ID NO: 22); the CDRH3 Domain has the amino acid sequence YYYKYDVGGFDY (SEQ ID NO: 23); the CDRL1 Domain has the amino acid sequence SVSSSISSSNLH (SEQ ID NO: 24); the CDRL2 Domain has the amino acid sequence GTSNLAS (SEQ ID NO: 25); and the CDRL3 Domain has the amino acid sequence QQWSSYPFT (SEQ ID NO: 26).

In certain embodiments, the Heavy Chain Variable Domain of the anti-LAG-3 antibody or fragment thereof comprises an amino acid sequence which is at least 75%, 80%, 85%, 90%, 95%, 99%, or 100% identical to: QVQMVQSGAEVKKPGASVKVSCKASGFNIKDTYIHWVRQAPGQGLEWMGEIDPANDN TKYDPKFQGRVTITADTSTSTVYMELSSLRSEDTAVYYCATYYYKYDVGGFDYWGQGT LVTVSS (SEQ ID NO: 27). In certain embodiments, the Light Chain Variable Domain of the anti-LAG-3 antibody or fragment thereof comprises an amino acid sequence which is at least 75%, 80%, 85%, 90%, 95%, 99%, or 100% identical to: EIVLTQSPGTL SLSPGERATLSCSVSSSISSSNLHWYQQKPGQAPRLLIYGTSNLASGIPDR FSGSGSGTDFTLTISRLEPEDFAVYYCQQWSSYPFTFGQGTKVEIK (SEQ ID NO: 28). In certain embodiments, the Heavy Chain Variable Domain of the anti-LAG-3 antibody or fragment thereof comprises an amino acid sequence which is at least 75%, 80%, 85%, 90%, 95%, 99%, or 100% identical to: QVQMVQSGAEVKKPGASVKVSCKASGFNIKDTYIHWVRQAPGQGLEWMGEIDPANDN TKYDPKFQGRVTITADTSTSTVYMEL SSLRSEDTAVYYCATYYYKYDVGGFDYWGQGT LVTVSS (SEQ ID NO: 27); and the Light Chain Variable Domain of the anti-LAG-3 antibody or fragment thereof comprises an amino acid sequence which is at least 75%, 80%, 85%, 90%, 95%, 99%, or 100% identical to:

(SEQ ID NO: 28)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIK.

In certain embodiments, the anti-LAG-3 antibody or fragment thereof comprises a Heavy Chain (HC) sequence which is at least 75%, 80%, 85%, 90%, 95%, 99%, or 100% identical to: QVQMVQSGAEVKKPGASVKVSCKASGFNIKDTYIHWVRQAPGQGLEWMGEIDPANDN TKYDPKFQGRVTITADTSTSTVYMEL SSLRSEDTAVYYCATYYYKYDVGGFDYWGQGT LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPCPA PELLGGPSVFLFPPKPKDTLMISRTPEVTCVVVDVSSHEDPEVKFNWYVDGVEVHNAKTK PREEQYASTYRVVSVLTVLHQDWLNGKEYKCKVSNKALPAPIEKTISKAKGQPREPQV YTLPPSREEMTKNQVSLTCLVKGFYPSDIAVEWESNGQPENNYKTTPPVLDSDGSFFLYS KLTVDKSRWQQGNVFSCSVMHEALHNHYTQKSLSLSPG (SEQ ID NO: 29). In certain embodiments, the anti-LAG-3 antibody or fragment thereof comprises a Light Chain (LC) sequence which is at least 75%, 80%, 85%, 90%, 95%, 99%, or 100% identical to: EIVLTQSPGTL SLSPGERATLSCSVSSSISSSNLHWYQQKPGQAPRLLIYGTSNLASGIPDR FSGSGSGTDFTLTISRLEPEDFAVYYCQQWSSYPFTFGQGTKVEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 30). In certain embodiments, the anti-LAG-3 antibody or fragment thereof comprises a Heavy Chain (HC) sequence which is at least 75%, 80%, 85%, 90%, 95%, 99%, or 100% identical to: QVQMVQSGAEVKKPGASVKVSCKASGFNIKDTYIHWVRQAPGQGLEWMGEIDPANDN TKYDPKFQGRVTITADTSTSTVYMEL SSLRSEDTAVYYCATYYYKYDVGGFDYWGQGT LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPCPA PELLGGPSVFLFPPKPKDTLMISRTPEVTCVVVDVSHEDPEVKFNWYVDGVEVHNAKTK PREEQYASTYRVVSVLTVLHQDWLNGKEYKCKVSNKALPAPIEKTISKAKGQPREPQV YTLPPSREEMTKNQVSLTCLVKGFYPSDIAVEWESNGQPENNYKTTPPVLDSDGSFFLYS KLTVDKSRWQQGNVFSCSVMHEALHNHYTQKSLSLSPG (SEQ ID NO: 29); and the anti-LAG-3 antibody or fragment thereof comprises a Light Chain (LC) sequence which is at least 75%, 80%, 85%, 90%, 95%, 99%, or 100% identical to:

(SEQ ID NO: 30)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In certain embodiments, the anti-LAG-3 antibody or fragment thereof comprises an Fc Region that is of the IgG1, IgG2, IgG3, IgG4, IgA1, or IgA2 isotype. In certain embodiments, the anti-LAG-3 antibody or fragment thereof comprises an Fc Region that is of a human IgG1 isotype, wherein the amino acid sequence of the IgG1 heavy chain constant region comprises: an N297A mutation, numbered according to the EU numbering system; or an N297Q mutation, numbered according to the EU numbering system. In certain embodiments, the anti-LAG-3 antibody or fragment thereof comprises an Fc Region of a human IgG4 isotype, wherein the amino acid sequence of the IgG4 heavy chain constant region comprises: an S228P mutation, numbered according to the EU numbering system; or an N297Q mutation, numbered according to the EU numbering system. In certain embodiments, the anti-LAG-3 antibody or fragment thereof is antagonistic to human LAG-3. In certain embodiments, the anti-LAG-3 antibody or fragment thereof deactivates, reduces, or inhibits an activity of human LAG-3. In certain embodiments, the anti-LAG-3 antibody or fragment thereof inhibits binding of human LAG-3 to MEW class II.

In certain embodiments, the anti-PD-1 active agent is administered before the anti-TIM-3 active agent and the anti-LAG-3 active agent. In certain embodiments, the anti-TIM-3 active agent is administered before the anti-LAG-3 active agent. In certain embodiments, the anti-LAG-3 active agent is administered before the anti-TIM-3 active agent. In certain embodiments, the anti-TIM-3 active agent and the anti-LAG-3 active agent are administered concurrently.

In certain embodiments, the anti-PD-1 active agent is administered in a pharmaceutical composition comprising: acetate, sucrose, polysorbate 80 (“PS80”), and water, and has a pH of about 4.0 to about 6.5; optionally wherein the concentration of the anti-PD-1 agent in the pharmaceutical composition is about 10 mg/mL to about 100 mg/mL In certain embodiments, the anti-TIM-3 active agent is administered in a pharmaceutical composition comprising: sodium citrate, sucrose, arginine, polysorbate 80, and has a pH 6.0; optionally wherein the concentration of the anti-TIM-3 agent in the pharmaceutical composition is about 50 mg/mL. In certain embodiments, the anti-LAG-3 active agent is administered in a pharmaceutical composition comprising: sodium acetate, trehalose, polysorbate 80, and has a pH 5.5; optionally wherein the concentration of the anti-LAG-3 agent in the pharmaceutical composition is about 50 mg/mL.

In certain embodiments, the present disclosure provides methods, combination therapies, uses, or kits for the treatment of cancer in a subject. In certain embodiments, the cancer comprises a tumor. In certain embodiments, the cancer comprises a locally advanced tumor, a metastatic solid tumor, or a combination thereof. In certain embodiments, the cancer comprises a tumor for which a PD-1 inhibitor is indicated.

In certain embodiments, the subject has received prior treatment with at least one anti-PD-1/anti-PDL-1 therapy. In certain embodiments, the subject has a cancer which failed a prior PD-1/PDL-1 inhibitor therapy. In certain embodiments, the subject has a cancer which continued to progress during a prior PD-1/PDL-1 inhibitor therapy. In certain embodiments, the cancer comprises a tumor that has acquired resistance to anti-PD-1 therapy. In certain embodiments, the cancer comprises a tumor that has innate resistance to anti-PD-1 therapy. In certain embodiments, the cancer comprises a tumor that has acquired resistance to anti-PD-1 therapy and an innate resistance to anti-PD-1 therapy. In certain embodiments, the tumor has a minimum LAG-3 expression of greater than or equal to 5% of LAG-3-positive immune cells (e.g., lymphocytes and macrophages) relative to all nucleated cells within the tumor region as shown by an appropriate immunohistochemical assay. The tumor region includes tumor cells, intratumoral stroma, and peritumoral stroma, with normal and/or adjacent uninvolved tissues not included. Suitable LAG-3 immunohistochemical assays are found in Wojcik et al., Consistent Measurement of LAG-3 expression Across Multiple Staining Platforms with the 17B4 Antibody Clone, bioRxiv (Feb. 22, 2022), doi.org/10.1101/2022.02.21.481075; and Johnson et al., Development of a LAG-3 Immunohistochemistry Assay for Melanoma, bioRxiv (Feb. 26, 2022), doi.org/10.1101/2022.02.25.481964.

In certain embodiments, the cancer is melanoma. In certain embodiments, the cancer is unresectable and/or metastatic melanoma. In certain embodiments, the cancer is squamous cell carcinoma of the head and neck (SCCHN). In certain embodiments, the cancer is recurrent or metastatic PD-L1+ SCCHN. In certain embodiments, the cancer is endometrial cancer. In certain embodiments, the cancer is MSI-H advanced endometrial cancer. In certain embodiments, the cancer is metastatic endometrial cancer. In certain embodiments, the cancer is endometrial cancer with evidence of disease progression on or following platinum-based chemotherapy and previous PD-L1 inhibitory therapy.

In certain embodiments, the administration or treatment of the present disclosure produces at least one therapeutic effect. In certain embodiments, the therapeutic effect is a reduction in size of a tumor. In certain embodiments, the therapeutic effect is a reduction in number of metastasic lesions over time. In certain embodiments, the therapeutic effect is a complete response (e.g., disappearance of all target lesions according to RECIST v1.1 criteria). In certain embodiments, the therapeutic effect is a partial response (e.g., ≥30% decrease in sum of target lesion diameters, according to RECIST v1.1 criteria). In certain embodiments, the therapeutic effect is a stable disease (e.g., ≤30% decrease and ≤20% increase in sum of target lesion diameters, according to RECIST v1.1 criteria). In certain embodiments, the therapeutic effect is a progressive disease (e.g., ≥20% increase in sum of target lesion diameters, according to RECIST v1.1 criteria). In certain embodiments, the therapeutic effect is evaluated according to RECIST v1.1 criteria.

### BRIEF DESCRIPTION OF THE FIGURES

FIG. 1 provides the results for best percentage change from baseline in target lesion size (sum of diameters) among individual patients assessable for response, according to certain combination therapy treatments of the present disclosure.

### DETAILED DESCRIPTION

### I. Active Agents and Compositions

### Antigen Binding Proteins and Binding Domains

In certain embodiments, the active agents of the present disclosure (e.g., anti-PD-1 active agent, anti-TIM-3 active agent, anti-LAG-3 active agent) comprise an antigen binding protein, such as an antibody or an antigen binding—fragment of an antibody. In certain embodiments, the active agents of the present disclosure (e.g., anti-PD-1 active agent, anti-TIM-3 active agent, anti-LAG-3 active agent) comprise an antibody or an antigen binding-fragment of an antibody.

In certain embodiments, antibodies of the present disclosure comprise: monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above.

In certain embodiments, the antibodies or antibody fragments comprise immunoglobulin molecules or immunologically active fragments of immunoglobulin molecules, i.e., molecules that contain an antigen-binding site. Immunoglobulin molecules can be of any type (e.g., IgG, IgE, IgM, IgD, IgA, and IgY), any class (e.g., IgG1, IgG2, IgG3, IgG4, IgA1, and IgA2) or subclass (e.g., IgG2a or IgG2b). In certain embodiments, the antibodies or antibody fragments are used as a therapeutic agent.

In certain embodiments, an antibody of the present disclosure comprises a monoclonal antibody (mAb). Monoclonal antibodies generally refer to a homogeneous antibody designed to be directed against a single epitope target or antigenic site, and which are generally generated from the immortalization and singular cloning of a plasma B-cell. In certain embodiments, monoclonal antibodies and monoclonal antibody variants of the present disclosure comprise intact monoclonal antibodies, full-length monoclonal antibodies, antibody fragments (such as Fab, Fab′), single-chain variants (scFv), mutated antibodies, fusion proteins comprising a portion of a monoclonal antibody, humanized monoclonal antibodies, chimeric monoclonal antibodies, and any other modified configuration of an immunoglobulin molecule that comprises an antigen recognition site and the ability to bind a target antigen.

In certain embodiments, the antibody or fragment thereof are produced or sourced by any manner known to those with skill in the art (e.g., hybridoma, phage selection, recombinant expression, transgenic animals). In certain embodiments, antibodies are developed in mice, rats or rabbits. In certain embodiments, the antibodies are produced by immunizing an animal with an immunogenic collection of cells, cell extracts, or protein preparations that contain the desired epitope. Examples of immunogens include, but are not limited to, primary cells, cultured cell lines, cancerous cells, proteins, peptides, nucleic acids, or tissue. In certain embodiments, the immunogen cells may be used as immunogens by themselves or in combination with a non-denaturing adjuvant.

In certain embodiments, the antibody or fragment thereof are produced by sequencing an existing antibody (or equivalent antibody fragment or variant which targets a desired epitope) and then recombinantly producing the antibody by any means known in the art. In certain embodiments, an antibody is sequenced, and the polynucleotide sequence is then cloned into a vector for expression or propagation. In certain embodiments, the sequence encoding the antibody or antibody fragment of interest is maintained in a vector in a host cell, wherein the host cell can be expanded, frozen, and thawed as needed for future production and use.

Antibodies (such as IgG antibodies) are generally configured as a tetramer having two Light Chains complexed with two Heavy Chains. Each light chain contains a Light Chain Variable Domain (VL) and a Light Chain Constant Domain (CL). Each heavy chain contains a Heavy Chain Variable Domain (VH), three Heavy Chain Constant Domains (CH1, CH2 and CH3), and a hinge domain located between the CH1 and CH2 Domains. The amino-terminal (“N-terminal”) portion of each chain includes a Variable Domain (i.e., Variable Region) of amino acids primarily responsible for antigen recognition. The carboxy-terminal (“C-terminal”) portion of each chain defines the constant region, with light chains having a single Constant Domain and heavy chains usually having three Constant Domains and a Hinge Domain. In general, the structure of the light chains of an IgG molecule is (N-terminal)-VL-CL-(C-terminal). The Light Chain Variable Domain (VL) generally consists of the 90 to 115 N-terminal amino acids in a mature light chain sequence. In general, the structure of the heavy chains of an IgG molecule is (N-terminal)-VH-CH1-Hinge-CH2-CH3-(C-terminal). The Heavy Chain Variable Domain (VH) generally consists of the 110 to 125 N-terminal amino acids in a mature heavy chain sequence.

The Variable Domain of an antibody molecule generally comprises: (i) complementarity determining regions (CDR), which contain the residues that target and contact an epitope, and (ii) non-CDR framework segments (FR), which maintain the structure of the domain and determine the positioning of the CDR loops (i.e., to permit the CDR loops to contact the target). VL domains generally have the structure (N-terminal)-FR1-CDRL1-FR2-CDRL2-FR3-CDRL3-FR4-(C-terminal). VH domains generally have the structure (N-terminal)-FR1-CDRH1-FR2-CDRH2-FR3-CDRH3-FR4-(C-terminal). The terms CDRL1 Domain, CDRL2 Domain, CDRL3 Domain, CDRH1 Domain, CDRH2 Domain, and CDRH3 Domain are generally directed to polypeptides that can be incorporated into a protein to bind to an epitope (including antibodies, antibody fragments, or antibody variants such as a single-chain binding molecule or other types of proteins). In certain embodiments, the antibody or fragment thereof comprises an epitope-binding site. In certain embodiments, the epitope-binding site may contain 1, 2, 3, 4, 5, or all 6 of the CDR Domains of such antibody.

In certain embodiments, the antibody or fragment thereof are humanized (i.e., retaining the sequence of the antigen-binding portion of a non-human antibody, and mutating the non-human remainder of the antibody with human antibody sequences). In general, a humanized antibody refers to a molecule (generally prepared using recombinant techniques known in the art) which has: (i) an antigen-binding site of an immunoglobulin from a non-human species, and (ii) a remaining immunoglobulin structure that is based upon the structure and/or sequence of a human immunoglobulin. Without being bound by theory, the general principle in humanizing an antibody involves retaining the basic sequence of the antigen-binding portion of the antibody, and then replacing the remainder of the non-human antibody with human antibody sequences. The general steps to humanize a monoclonal antibody are well known in the art, including U.S. Pat. Nos. 4,816,567; 5,807,715; 5,866,692; and 6,331,415. In certain embodiments, humanized antibodies preserve all CDR sequences (e.g., a humanized mouse antibody which contains all six CDRs from the mouse antibodies). In certain embodiments, humanized antibodies have one or more CDRs (one, two, three, four, five, or six) which differ in sequence relative to the original antibody.

### Programmed Death-1 (PD-1) Active Agent

Programmed Death-1 protein (PD-1, also known as PD1 or CD279) is an approximately 31 kD type I membrane protein member of the extended CD28/CTLA-4 family of T-cell regulators. PD-1 is expressed on activated T-cells, B-cells, monocytes, and (at low levels) in natural killer (NK) T-cells. The extracellular region of PD-1 comprises a single immunoglobulin (Ig) V domain (with about 23% identity to the equivalent domain in CTLA-4) which is followed by a transmembrane region and an intracellular tail that contains two phosphorylation sites located in an immunoreceptor tyrosine-based inhibitory motif and an immunoreceptor tyrosine-based switch motif.

PD-1 has been shown to broadly negatively regulate immune response, including mediating inhibition of the immune system by binding to B7-H1 and B7-DC (see, e.g., US 2005/0059051; US 2007/0202100; US 2008/0311117; US 2009/00110667; US 2009/0274666; US 2009/0313687; US 2009/0055944; U.S. Pat. No. 10,577,422). The role of B7-H1 and PD-1 in inhibiting T-cell activation and proliferation has led to studies around the role these biomolecules might serve as therapeutic targets for treatments of infection, inflammation, tumors, and cancers (see, e.g., US 2004/0241745; US 2008/0311117; US 2009/0217401; US 2010/0028330; 2010/0040614; U.S. Pat. No. 10,577,422). Antibodies capable of binding to PD-1 have been studied and tested for therapeutic effectiveness in treating these indications. PD-1-targeting agents, such as retifanlimab, have also been shown to effectively sustain/restore the effector function of PD-1-expressing cells, including T-cells, by blocking checkpoint inhibitory interactions between PD-1 and its two primary ligands, PD-L1 and PD-L2. Retifanlimab has also been shown to disrupt the PD-1/PD-L1 inhibitory axis, and to enhance IFN-γ secretion in staphylococcal enterotoxin B (SEB)-stimulated human peripheral blood mononuclear cells with activity comparable to pembrolizumab and nivolumab replicas.

More than 30 clinical trials have been initiated or completed to study anti-PD-1 antibodies and combination therapies which include anti-PD-1 agents. However, despite continued study of PD-1-based compositions and therapies, a need remains for improved compositions and treatment regimens capable of effectively antagonizing and/or blocking PD-1/PDL-1 activation, proliferation, and inhibition of T-cell activation, which could provide improved therapeutic effect to patients suffering from cancer or corresponding diseases and conditions.

The present disclosure provides methods, combination therapies, uses and kits which comprise: a therapeutically effective amount of an anti-PD-1 active agent, a therapeutically effective amount of an anti-TIM-3 active agent, and a therapeutically effective amount of an anti-LAG-3 active agent.

In certain embodiments, the methods, combination therapies, uses, or kits of the present disclosure comprise an anti-PD-1 active agent. In certain embodiments, the anti-PD-1 active agent comprises an anti-PD-1 antibody, or PD-1 binding-fragment thereof.

In certain embodiments, the anti-PD-1 active agent is capable of binding to a continuous or discontinuous (e.g., conformational) portion (epitope) of human PD-1 (i.e., CD279). In certain embodiments, the anti-PD-1 active agent binds to PD-1 molecules of one or more non-human species, including primate species such as cynomolgus monkey. A representative human PD-1 polypeptide (NCBI Sequence NP_005009.2) is as follows:

(SEQ ID NO: 31)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

In certain embodiments, the anti-PD-1 antibody is INCMGA00012 (i.e., retifanlimab, MGA012).

In certain embodiments, the anti-PD-1 active agent comprises an anti-PD-1 antibody, a PD-1-binding molecule, or a PD-1 binding-fragment described in U.S. Pat. No. 10,577,422, which is incorporated herein by reference as related to anti-PD-1 active agents, anti-PD-1 antibodies, PD-1-binding molecules, and PD-1 binding-fragments for use in the treatment of cancer, as well as corresponding methods, uses, pharmaceutical compositions, treatments, and production methods therein.

In certain embodiments, the anti-PD-1 antibody is nivolumab, also known as BMS-936558 or MDX1106 (Bristol-Myers Squibb). In certain embodiments, the anti-PD-1 antibody is pembrolizumab, also known as lambrolizumab or MK-3475 (Merck & Co). In certain embodiments, the anti-PD-1 antibody is pidilizumab, also known as CT-011 (CureTech). In certain embodiments, the anti-PD-1 antibody is MEDI0680, also known as AMP-514 (Medimmune). In certain embodiments, the anti-PD-1 antibody is PDR001 (Novartis). In certain embodiments, the anti-PD-1 antibody is REGN2810 (Regeneron). In certain embodiments, the anti-PD-1 antibody is PF-06801591 (Pfizer). In certain embodiments, the anti-PD-1 antibody is BGB-A317 (BeiGene). In certain embodiments, the anti-PD-1 antibody is TSR-042 (AnaptysBio). In certain embodiments, the anti-PD-1 antibody is SHR-1210 (Hengrui).

In certain embodiments, the anti-PD-1 active agent comprises a PD-1 binding-fragment of an anti-PD-1 antibody, including (but not limited to) immunoconjugates, diabodies, BiTEs, bispecific antibodies, antibody-bonding fragments, and similar molecules. In certain embodiments, the anti-PD-1 active agent is an anti-PD-1 antibody from U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,920,075; 8,993,731; 9,102,727; 9,205,148; 9,815,897; 9,982,053; 10,414,821; 10,066,013; 10,077,305; 10,160,806; US 2013/0202623 A1; US 2013/0291136 A1; US 2014/0044738 A1; US 2014/0356363 A1; US 2016/0145355 A1; US 2016/0075783 A1; and US 2019/0309069 A1; each being incorporated herein by reference as related to anti-PD-1 active agents for use treating diseases, such as cancer.

In certain embodiments, the anti-PD-1 antibody or fragment thereof comprises an Fc Region that is of the IgG1, IgG2, IgG3, or IgG4 isotype. In certain embodiments, the anti-PD-1 antibody or fragment thereof comprises an Fc Region that is of the IgG4 isotype. In certain embodiments, the antibody comprises a Hinge Domain of the IgG4 isotype that comprises a stabilizing mutation. In certain embodiments, the anti-PD-1 antibody or fragment thereof comprises a variant Fc Region that comprises: (A) one or more amino acid modifications that reduce the affinity of the variant Fc Region for an FcγR, wherein the one or more modifications that reduce the affinity of the variant Fc Region for an FcγR comprise the substitution of L234A, L235A, or L234A+L235A, wherein the numbering is that of the EU index as in Kabat; and/or (B) one or more amino acid modifications that enhance the serum half-life of the variant Fc Region, wherein the one or more modifications that enhance the serum half-life of the variant Fc Region comprise the substitution of M252Y, M252Y+S254T, M252Y+T256E, M252Y+S254T+T256E, or K288D+H435K, wherein the numbering is that of the EU index as in Kabat.

In certain embodiments, the anti-PD-1 active agent is characterized by one or more of the following criteria: (1) binds human PD-1 as endogenously expressed on the surface of a stimulated human T-cell; (2) binds human PD-1 with an equilibrium binding constant (K_{D}) of 40 nM or less; (3) binds human PD-1 with an equilibrium binding constant (K_{D}) of 5 nM or less; (4) binds human PD-1 with an on rate (kₐ) of 1.5×10⁴ M⁻¹ min⁻¹ or more; (5) binds human PD-1 with an on rate (kₐ) of 90.0×10⁴ M⁻¹ min⁻¹ or more; (6) binds human PD-1 with an off rate (k_{d}) of 7×10⁴ min⁻¹ or less; (7) binds human PD-1 with an off rate (k_{d}) of 2×10⁴ min⁻¹ or less; (8) binds non-human primate PD-1 (e.g., PD-1 of cynomolgus monkey); (9) inhibits (i.e., blocks or interferes with the binding/the inhibitory activity) of PD-1 ligand (PDL-1/PDL-2) to PD-1; (10) stimulates an immune response; and/or (11) synergizes with an anti-human LAG-3 and/or anti-human TIM-3 antibody to stimulate an antigen specific T-cell response.

In certain embodiments, the anti-PD-1 active agent is administered intravenously. In certain embodiments, the anti-PD-1 active agent is administered intravenously once every three weeks or once every four weeks. In certain embodiments, the anti-PD-1 active agent is administered intravenously once every three weeks. In certain embodiments, the anti-PD-1 active agent is administered intravenously once every four weeks.

### PD-1 CDRs

In certain embodiments, the anti-PD-1 active agent comprises an anti-PD-1 antibody, or PD-1 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain.

In certain embodiments, the CDRH1 Domain has the amino acid sequence SYWMN (SEQ ID NO: 1).

In certain embodiments, the CDRH2 Domain has the amino acid sequence VIHIPSDSETWLDQKFK (SEQ ID NO: 2).

(SEQ ID NO: 2)
VIHPSDSETWLDQKFK.

In certain embodiments, the CDRH3 Domain has the amino acid sequence

(SEQ ID NO: 3)
EHYGTSPFAY.

In certain embodiments, the CDRL1 Domain has the amino acid sequence

(SEQ ID NO: 4)
RASESVDNYGMSFMNW.

In certain embodiments, the CDRL2 Domain has the amino acid sequence AASNQGS (SEQ TD NO: 5). In certain embodiments, the CDRL2 Domain has the amino acid sequence AASNRGS (SEQ ID NO: 32).

In certain embodiments, the CDRL3 Domain has the amino acid sequence

(SEQ ID NO: 6)
QQSKEVPYT.

### PD-1 VHs and VLs

In certain embodiments, the Heavy Chain Variable Domain (VH) of the anti-PD-1 antibody or fragment thereof comprises an amino acid sequence which is at least 75%, 80%, 85%, 90%, 95%, 99%, or 100% identical to: QVQLQQPGAELVRPGASVKLSCKASGYSFTSYWMNWVKQRPGQGLEWIGVIIIPSDSET WLDQKFKDKATLTVDKSSTTAYMQLISPTSEDSAVYYCAREHYGTSPFAYWGQGTLVT VSS (SEQ ID NO: 33). In certain embodiments, the Heavy Chain Variable Domain (VH) of the anti-PD-1 antibody or fragment thereof is encoded by a polynucleotide sequence that is least 75%, 80%, 85%, 90%, 95%, 99%, or 100% identical to:

(SEQ ID NO: 34)
gaggtccaactgcagcagcctggggctgaactggtgaggcctggagctt
cagtgaagctgtcctgcaaggcttctggctactccttcaccagctactg
gatgaactgggtgaagcagaggcctggacaaggccttgagtggattggc
gtgattcatccttccgatagtgaaacttggttagatcagaagttcaagg
acaaggccacattgactgtagacaaatcctccaccacagcctacatgca
actcatcagcccgacatctgaggactctgcggtctattactgtgcaagg
gagcactacggtactagcccgtttgcttactggggccaagggactctgg
tcactgtgtcttcc.

In certain embodiments, the Heavy Chain Variable Domain (VH) of the anti-PD-1 antibody or fragment thereof comprises a humanized amino acid sequence (e.g., hPD-1 mAb).

In certain embodiments, the Heavy Chain Variable Domain (VH) of the anti-PD-1 antibody or fragment thereof comprises an amino acid sequence which is at least 75%, 80%, 85%, 90%, 95%, 99%, or 100% identical to: QVQLVQSGAEVKKPGASVKVSCKASGYSFTSYWMNWVRQAPGQGLEWIGVIIIPSDSE TWLDQKFKDRVTITVDKSTSTAYMELSSLRSEDTAVYYCAREHYGTSPFAYWGQGTLV TVSS (SEQ ID NO: 7). In certain embodiments, the Heavy Chain Variable Domain (VH) of the anti-PD-1 antibody or fragment thereof is encoded by a polynucleotide sequence that is least 75%, 80%, 85%, 90%, 95%, 99%, or 100% identical to:

(SEQ ID NO: 35)
caagttcaattggtacagagcggggcagaggtgaagaaacccggcgcca
gtgttaaggtgtcctgcaaagccagcggttacagctttacaagctattg
gatgaattgggtgcgtcaagcaccagggcagggtctggaatggattggg
gtgatacatccttctgacagcgaaacatggttggaccagaaatttaaag
atcgtgtgacaattacagtcgataagtccacaagcactgcttacatgga
actctccagcttgcggtccgaggacaccgctgtgtattattgcgccaga
gagcactacggcacatcaccttttgcatactggggccagggaactctcg
taaccgtatcctcc.

In certain embodiments, the Heavy Chain Variable Domain (VH) of the anti-PD-1 antibody or fragment thereof comprises an amino acid sequence which is at least 75%, 80%, 85%, 90%, 95%, 99%, or 100% identical to: QVQLVQSGAEVKKPGASVKVSCKASGYSFTSYWMNWVRQAPGQGLEWAGVIHPSDSE TWLDQKFKDRVTITVDKSTSTAYMELSSLRSEDTAVYYCAREHYGTSPFAYWGQGTLV TVSS (SEQ ID NO: 36). In certain embodiments, the Heavy Chain Variable Domain (VH) of the anti-PD-1 antibody or fragment thereof is encoded by a polynucleotide sequence that is least 75%, 80%, 85%, 90%, 95%, 99%, or 100% identical to:

(SEQ ID NO: 37)
caagttcaattggtacagagcggggcagaggtgaagaaacccggcgcca
gtgttaaggtgtcctgcaaagccagcggttacagctttacaagctattg
gatgaattgggtgcgtcaagcaccagggcagggtctggaatgggctggg
gtgatacatccttctgacagcgaaacatggttggaccagaaatttaaag
atcgtgtgacaattacagtcgataagtccacaagcactgcttacatgga
actctccagcttgcggtccgaggacaccgctgtgtattattgcgccaga
gagcactacggcacatcaccttttgcatactggggccagggaactctcg
taaccgtatcctcc.

In certain embodiments, the Light Chain Variable Domain (VL) of the anti-PD-1 antibody or fragment thereof comprises an amino acid sequence which is at least 75%, 80%, 85%, 90%, 95%, 99%, or 100% identical to: DIVLTQSPASLAVSLGQRATISCRANESVDNYGMSFMNWFQQKPGQPPKLLIHAASNQG SGVPARFSGSGFGTDFSLNIHPMEEDDAAMYFCQQSKEVPYTFGGGTKLEIK (SEQ ID NO: 38). In certain embodiments, the Light Chain Variable Domain (VL) of the anti-PD-1 antibody or fragment thereof is encoded by a polynucleotide sequence that is least 75%, 80%, 85%, 90%, 95%, 99%, or 100% identical to:

(SEQ ID NO: 39)
gacattgtgctgacccaatctccagcttctttggctgtgtctctagggc
agagggccaccatctcctgcagagccaacgaaagtgttgataattatgg
catgagttttatgaactggttccaacagaaaccaggacagccacccaaa
ctcctcatccatgctgcatccaaccaaggatccggggtccctgccaggt
ttagtggcagtgggtttgggacagacttcagcctcaacatccatcctat
ggaggaggatgatgctgcaatgtatttctgtcagcaaagtaaggaggtt
ccgtacacgttcggaggggggaccaagctggaaataaaa.

In certain embodiments, the Light Chain Variable Domain (VL) of the anti-PD-1 antibody or fragment thereof comprises a humanized amino acid sequence (e.g., hPD-1 mAb).

In certain embodiments, the Light Chain Variable Domain (VL) of the anti-PD-1 antibody or fragment thereof comprises an amino acid sequence which is at least 75%, 80%, 85%, 90%, 95%, 99%, or 100% identical to: EIVLTQSPATL SLSPGERATLSCRASESVDNYGMSFMNWFQQKPGQPPKLLIHAASNQGS GVPSRFSGSGSGTDFTLTISSLEPEDFAVYFCQQSKEVPYTFGGGTKVEIK (SEQ ID NO: 8). In certain embodiments, the Light Chain Variable Domain (VL) of the anti-PD-1 antibody or fragment thereof is encoded by a polynucleotide sequence that is least 75%, 80%, 85%, 90%, 95%, 99%, or 100% identical to:

(SEQ ID NO: 40)
gaaatcgtactcacccagtcacctgcaaccctttctctgagccccggtg
aacgtgccactctcagctgcagagcaagtgagagtgtggacaattacgg
catgtccttcatgaactggtttcagcagaagcctgggcagccacctaag
ctgctcatccacgccgcctctaaccagggatctggggtgccttcacgtt
tttctggatcaggaagtggcactgacttcacccttacaatcagctctct
ggagccagaggactttgccgtctatttctgccagcaatctaaagaggtg
ccctatacttttggtggcgggaccaaggttgagatcaaa.

In certain embodiments, the Light Chain Variable Domain (VL) of the anti-PD-1 antibody or fragment thereof comprises an amino acid sequence which is at least 75%, 80%, 85%, 90%, 95%, 99%, or 100% identical to EIVLTQSPATL SLSPGERATLSCRANESVDNYGMSFMNWFQQKPGQPPKLLIHAASNQG SGVPSRFSGSGSGTDFTLTISSLEPEDFAVYFCQQSKEVPYTFGGGTKVEIK (SEQ ID NO: 41). In certain embodiments, the Light Chain Variable Domain (VL) of the anti-PD-1 antibody or fragment thereof is encoded by a polynucleotide sequence that is least 75%, 80%, 85%, 90%, 95%, 99%, or 100% identical to:

(SEQ ID NO: 42)
gaaatcgtactcacccagtcacctgcaaccctttctctgagccccggtg
aacgtgccactctcagctgcagagcaaatgagagtgtggacaattacgg
catgtccttcatgaactggtttcagcagaagcctgggcagccacctaag
ctgctcatccacgccgcctctaaccagggatctggggtgccttcacgtt
tttctggatcaggaagtggcactgacttcacccttacaatcagctctct
ggagccagaggactttgccgtctatttctgccagcaatctaaagaggtg
ccctatacttttggtggcgggaccaaggttgagatcaaa.

In certain embodiments, the Light Chain Variable Domain (VL) of the anti-PD-1 antibody or fragment thereof comprises an amino acid sequence which is at least 75%, 80%, 85%, 90%, 95%, 99%, or 100% identical to EIVLTQSPATL SLSPGERATLSCRASESVDNYGMSFMNWFQQKPGQPPKLLIHAASNRGS GVPSRFSGSGSGTDFTLTISSLEPEDFAVYFCQQSKEVPYTFGGGTKVEIK (SEQ ID NO: 43). In certain embodiments, the Light Chain Variable Domain (VL) of the anti-PD-1 antibody or fragment thereof is encoded by a polynucleotide sequence that is least 75%, 80%, 85%, 90%, 95%, 99%, or 100% identical to:

(SEQ ID NO: 44)
gaaatcgtactcacccagtcacctgcaaccctttctctgagccccggtg
aacgtgccactctcagctgcagagcaagtgagagtgtggacaattacgg
catgtccttcatgaactggtttcagcagaagcctgggcagccacctaag
ctgctcatccacgccgcctctaaccgcggatctggggtgccttcacgtt
tttctggatcaggaagtggcactgacttcacccttacaatcagctctct
ggagccagaggactttgccgtctatttctgccagcaatctaaagaggtg
ccctatacttttggtggcgggaccaaggttgagatcaaa.

### PD-1 HC and LC

In certain embodiments, the anti-PD-1 antibody or fragment thereof comprises a Heavy Chain (HC) sequence which is at least 75%, 80%, 85%, 90%, 95%, 99%, or 100% identical to:

(SEQ ID NO: 9)
QVQLVQSGAEVKKPGASVKVSCKASGYSFTSYWMNWVRQAPGQGLEWIG
VIHPSDSETWLDQKFKDRVTITVDKSTSTAYMELSSLRSEDTAVYYCAR
EHYGTSPFAYWGQGTLVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLG.

In certain embodiments, the anti-PD-1 antibody or fragment thereof comprises a Light Chain (LC) sequence which is at least 75%, 80%, 85%, 90%, 95%, 99%, or 100% identical to:

(SEQ ID NO: 10)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

### T-Cell Immunoglobulin and Mucin Domain-3 (TIM-3) Active Agent

T-cell immunoglobulin and mucin domain-3 (TIM-3, also known as TIM3) is a type I membrane protein in the immunoglobulin (Ig) superfamily. TIM-3 is expressed on activated T-helper type 1 (Th1) and CD8+ T (Tc1) lymphocytes, macrophages, activated natural killer (NK) cells, and IL-17-producing Th17 cells. The extracellular region of TIM-3 comprises an extracellular Ig variable-like (IgV) domain, an extracellular mucin-like domain, and a cytoplasmic domain with six conserved tyrosine residues.

TIM-3 has been shown to function in inhibiting T-cell, myeloid cell, and NK cell-mediated responses, as well as promoting immunological tolerance. For example, the binding of a neutralizing immunoglobulin domain to TIM-3 IgV peptide ligands caused hyperproliferation of Th1 cells and Th1 cytokine release in immunized mice. Upregulation of TIM-3 expression in CD8+ T-cells has also been found in cancer patients. For example, approximately 30% of NY-ESO-1-specific CD8+ T-cells in patients with advanced melanoma exhibited upregulation of TIM-3 expression (see Fourcade et al., J Exp Med 207:2175-86 (2010)).

The correlation of TIM-3 with T-cell related activity and proliferation has led to studies around the role these biomolecules might serve as therapeutic targets for treatments of related diseases, including cancers. Specifically, antibodies capable of binding to TIM-3 have been studied and tested for therapeutic effectiveness in treating these indications. However, despite continued study of TIM-3-based compositions and therapies, a need remains for improved compositions and treatment regimens capable of effectively antagonizing and/or blocking TIM-3 activation, proliferation, and modulation of T-cell activation, which could provide improved therapeutic effect to patients suffering from cancer or corresponding diseases and conditions.

The present disclosure provides methods, combination therapies, uses and kits which comprise: a therapeutically effective amount of an anti-PD-1 active agent, a therapeutically effective amount of an anti-TIM-3 active agent, and a therapeutically effective amount of an anti-LAG-3 active agent.

In certain embodiments, the methods, combination therapies, uses, or kits of the present disclosure comprise an anti-TIM-3 active agent. In certain embodiments, the anti-TIM-3 active agent comprises an anti-TIM-3 antibody, or TIM-3 binding-fragment thereof.

In certain embodiments, the anti-TIM-3 active agent is capable of binding to a continuous or discontinuous (e.g., conformational) portion (epitope) of human TIM-3. In certain embodiments, the anti-TIM-3 active agent binds to TIM-3 molecules of one or more non-human species, including primate species such as cynomolgus monkey.

A representative human TIM-3 polypeptide (Swiss-Prot accession number Q8TDQ0-1) is as follows:

(SEQ ID NO: 45)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTRQRDFTAAFPRMLTTRGHGPAETQTLGSL
PDINLTQISTLANELRDSRLANDLRDSGATIRIGIYIGAGICAGLALAL
IFGALIFKWYSHSKEKIQNLSLISLANLPPSGLANAVAEGIRSEENIYT
IEENVYEVEEPNEYYCYVSSRQQPSQPLGCRFAMP.

In certain embodiments, the anti-TIM-3 antibody is Antibody A. As used herein, “Antibody A” describes an antibody which includes the following Heavy Chain (HC) and Light Chain (LC) sequences—the Heavy Chain has the amino acid sequence: EVQLVESGGGLVQPGGSLRLSCAASGFTFRQNAWSWVRRAPGKGLEWVSAISGSGGST YYADSVKGRFTISRDNSKNTLYLQMNSLRAEDTAVYYCAKGGDYGGNYFDYWGQGTL VTVSSASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKRVEPKSCDKTHTCPPCPAP ELLGGPSVFLFPPKPKDTLMISRTPEVTCVVVDVSSHEDPEVKFNWYVDGVEVHNAKTKP REEQYASTYRVVSVLTVLHQDWLNGKEYKCKVSNKALPAPIEKTISKAKGQPREPQVY TLPPSREEMTKNQVSLTCLVKGFYPSDIAVEWESNGQPENNYKTTPPVLDSDGSFFLYSK LTVDKSRWQQGNVFSCSVMHEALHNHYTQKSLSLSPG (SEQ ID NO: 19), and the Light Chain has the amino acid sequence:

(SEQ ID NO: 20)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In certain embodiments, the anti-TIM-3 active agent comprises an anti-TIM-3 antibody, a TIM-3-binding molecule, or a TIM-3 binding-fragment described in U.S. Pat. No. 10,639,368, which is incorporated herein by reference in its entirety as related to anti-TIM-3 active agents, anti-TIM-3 antibodies, a TIM-3-binding molecules, and a TIM-3 binding-fragments for use in the treatment of cancer, as well as corresponding methods, uses, pharmaceutical compositions, treatments, and production methods therein.

In certain embodiments, the anti-TIM-3 active agent comprises a TIM-3 binding-fragment of an anti-TIM-3 antibody, including (but not limited to) immunoconjugates, diabodies, BiTEs, bispecific antibodies, antibody-bonding fragments, and similar molecules.

In certain embodiments, the anti-TIM-3 antibody or fragment thereof comprises an Fe Region that is of the IgG1, IgG2, IgG3, IgG4, IgA1, or IgA2 isotype. In certain embodiments, the anti-TIM-3 antibody or fragment thereof comprises an Fe Region that is of the IgG1 isotype. In certain embodiments, the anti-TIM-3 antibody or fragment thereof comprises an Fe Region that is of the IgG1 isotype, wherein the amino acid sequence of the IgG1 heavy chain constant region comprises: an N297A mutation, numbered according to the EU numbering system; or an N297Q mutation, numbered according to the EU numbering system. In certain embodiments, the anti-TIM-3 antibody or fragment thereof comprises an Fe Region that is of the IgG4 isotype. In certain embodiments, the anti-TIM-3 antibody or fragment thereof comprises an Fe Region that is of the IgG4 isotype, wherein the amino acid sequence of the IgG4 heavy chain constant region comprises: an S228P mutation, numbered according to the EU numbering system; or an N297Q mutation, numbered according to the EU numbering system.

In certain embodiments, the anti-TIM-3 active agent is administered intravenously. In certain embodiments, the anti-TIM-3 active agent is administered intravenously once every two weeks, once every three weeks, or once every four weeks. In certain embodiments, the anti-TIM-3 active agent is administered intravenously once every two weeks. In certain embodiments, the anti-TIM-3 active agent is administered intravenously. In certain embodiments, the anti-TIM-3 active agent is administered intravenously once every three weeks. In certain embodiments, the anti-TIM-3 active agent is administered intravenously once every four weeks.

### TIM-3 CDRs

In certain embodiments, the anti-TIM-3 active agent comprises an anti-TIM-3 antibody, or TIM-3 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain.

In certain embodiments, the CDRH1 Domain has the amino acid sequence RQNAWS (SEQ ID NO: 11). In certain embodiments, the CDRH1 Domain has the amino acid sequence SSYAMS (SEQ ID NO: 46).

In certain embodiments, the CDRH2 Domain has the amino acid sequence

(SEQ ID NO: 12)
WVSAISGSGGSTY.

In certain embodiments, the CDRH3 Domain has the amino acid sequence

(SEQ ID NO: 13)
AKGGDYGGNYFD.

In certain embodiments, the CDRL1 Domain has the amino acid sequence X₁ASQSVSSSYLA (SEQ ID NO: 47, wherein X₁ is R or G. In certain embodiments, the CDRL1 Domain has the amino acid sequence X₁ASQSVSSYLA (SEQ ID NO: 48), wherein X₁ is R or G. In certain embodiments, the CDRL1 Domain has the amino acid sequence RASQSVSSYLA (SEQ ID NO: 14). In certain embodiments, the CDRL1 Domain has the amino acid sequence RASQSVSSSYLA (SEQ ID NO: 49).

In certain embodiments, the CDRL2 Domain has the amino acid sequence X₁ASX₂RAT (SEQ ID NO: 50), wherein X₁ is D or G, and X₂ is N, S, or T. In certain embodiments, the CDRL2 Domain has the amino acid sequence DASNRAT (SEQ ID NO: 15). In certain embodiments, the CDRL2 Domain has the amino acid sequence DASSRAT (SEQ ID NO: 51).

In certain embodiments, the CDRL3 Domain has the amino acid sequence QQYGSSPJT (SEQ ID NO: 52), wherein J is L or I. In certain embodiments, the CDRL3 Domain has the amino acid sequence QQYGSSPLT (SEQ ID NO: 16). In certain embodiments, the CDRL3 Domain has the amino acid sequence QQYGSSPIT (SEQ ID NO: 53).

### TIM-3 VHs and VLs

In certain embodiments, the Heavy Chain Variable Domain (VH) of the anti-TIM-3 antibody or fragment thereof comprises an amino acid sequence which is at least 75%, 80%, 85%, 90%, 95%, 99%, or 100% identical to:

(SEQ ID NO: 17)
EVQLVESGGGLVQPGGSLRLSCAASGFTFRQNAWSWVRRAPGKGLEWVS
AISGSGGSTYYADSVKGRFTISRDNSKNTLYLQMNSLRAEDTAVYYCAK
GGDYGGNYFDYWGQGTLVTVSS.

In certain embodiments, the Light Chain Variable Domain (VL) of the anti-TIM-3 antibody or fragment thereof comprises an amino acid sequence which is at least 75%, 80%, 85%, 90%, 95%, 99%, or 100% identical to:

(SEQ ID NO: 18)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIK.

### TIM-3 HC and LC

In certain embodiments, the anti-TIM-3 antibody or fragment thereof comprises a Heavy Chain (HC) sequence which is at least 75%, 80%, 85%, 90%, 95%, 99%, or 100% identical to:

(SEQ ID NO: 19)
EVQLVESGGGLVQPGGSLRLSCAASGFTFRQNAWSWVRRAPGKGLEWVS
AISGSGGSTYYADSVKGRFTISRDNSKNTLYLQMNSLRAEDTAVYYCAK
GGDYGGNYFDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYASTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

In certain embodiments, the anti-TIM-3 antibody or fragment thereof comprises a Light Chain (LC) sequence which is at least 75%, 80%, 85%, 90%, 95%, 99%, or 100% identical to:

(SEQ ID NO: 20)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

### Lymphocyte Activation Gene 3 (LAG-3) Active Agent

Lymphocyte activation gene 3 (LAG-3, also known as LAG3 and CD223) is a type I membrane protein in the immunoglobulin (Ig) superfamily. LAG-3 is expressed on activated effector T lymphocytes (Teff), activated regulatory T lymphocytes (Treg), activated B lymphocytes, resting natural killer (NK) cells, and resting plasmacytoid dendritic cells (PDC). The extracellular region of LAG-3 comprises four extracellular Ig domains and a cytoplasmic domain containing a conserved repeated EP motif and a single conserved KIEELE motif.

LAG-3 has been shown to function in the negative regulation of activated T-cells, specifically by targeting the MHC class II ligand expressed on antigen presenting cells (APC) and activated T-cells. The interaction between LAG-3 and MHC class II has been shown to inhibit proliferation and cytokine secretion of CD4+ and CD8+ Teff cells.

The correlation of LAG-3 with modulating immune response and corresponding T-cell related activity and proliferation has led to studies around the role these biomolecules might serve as therapeutic targets for treatments of related diseases, including cancers. Specifically, antibodies capable of binding to LAG-3 have been studied and tested for therapeutic effectiveness in treating these indications. However, despite continued study of LAG-3-based compositions and therapies, a need remains for improved compositions and treatment regimens capable of effectively antagonizing and/or blocking LAG-3 activation, proliferation, and modulation of T-cell activation, which could provide improved therapeutic effect to patients suffering from cancer or corresponding diseases and conditions.

The present disclosure provides methods, combination therapies, uses and kits which comprise: a therapeutically effective amount of an anti-PD-1 active agent, a therapeutically effective amount of an anti-TIM-3 active agent, and a therapeutically effective amount of an anti-LAG-3 active agent.

In certain embodiments, the methods, combination therapies, uses, or kits of the present disclosure comprise an anti-LAG-3 active agent. In certain embodiments, the anti-LAG-3 active agent comprises an anti-LAG-3 antibody, or LAG-3 binding-fragment thereof.

In certain embodiments, the anti-LAG-3 active agent is capable of binding to a continuous or discontinuous (e.g., conformational) portion (epitope) of human LAG-3. In certain embodiments, the anti-LAG-3 active agent binds to LAG-3 molecules of one or more non-human species, including primate species such as cynomolgus monkey.

A representative human LAG-3 polypeptide (GenBank accession number NM_002286.5) is as follows: VPVVWAQEGAPAQLPCSPTIPLQDLSLLRRAGVTWQHQPDSGPPAAAPGHPLAP GPHPAAPSSWGPRPRRYTVLSVGPGGLRSGRLPLQPRVQLDERGRQRGDFSLWLRPARR ADAGEYRAAVHLRDRALSCRLRLRLGQASMTASPPGSLRASDWVILNCSF SRPDRPASV HWFRNRGQGRVPVRESPHHHLAESFLFLPQVSPMD SGPWGCIL TYRDGFNVSIMYNLTV LGLEPPTPLTVYAGAGSRVGLPCRLPAGVGTRSFLTAKW TPPGGGPDLLVTGDNGDF TL RLEDVSQAQAGTYTCHIHLQEQQLNATVTLAIITVTPKSFGSPGSLGKLLCEVTPVSGQE RFVWSSLDTPSQRSFSGPWLEAQEAQLL SQPWQCQLYQGERLLGAAVYFTELSSPGAQR SGRAPGALPAGHLLLFLILGVLSLLLLVTGAFGFHLWRRQWRPRRF SALEQGIIIPPQAQS KIEELEQEPEPEPEPEPEPEPEPEPEQL (SEQ ID NO: 54).

In certain embodiments, the anti-LAG-3 antibody is Antibody B. As used herein, “Antibody B” describes an antibody which includes the following Heavy Chain (HC) and Light Chain (LC) sequences—the Heavy Chain has the amino acid sequence: QVQMVQSGAEVKKPGASVKVSCKASGFNIKDTYIHWVRQAPGQGLEWMGEIDPANDN TKYDPKFQGRVTITADTSTSTVYMEL SSLRSEDTAVYYCATYYYKYDVGGFDYWGQGT LVTVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKRVEPKSCDKTHTCPPCPA PELLGGPSVFLFPPKPKDTLMISRTPEVTCVVVDVSHEDPEVKFNWYVDGVEVHNAKTK PREEQYASTYRVVSVLTVLHQDWLNGKEYKCKVSNKALPAPIEKTISKAKGQPREPQV YTLPPSREEMTKNQVSLTCLVKGFYPSDIAVEWESNGQPENNYKTTPPVLDSDGSFFLYS KLTVDKSRWQQGNVFSCSVMHEALHNHYTQKSLSLSPG (SEQ ID NO: 29), and the Light Chain has the amino acid sequence:

(SEQ ID NO: 30)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In certain embodiments, the anti-LAG-3 active agent comprises an anti-LAG-3 antibody, a LAG-3-binding molecule, or a LAG-3 binding-fragment described in U.S. Pat. No. 10,844,119, which is incorporated herein by reference in its entirety as related to anti-LAG-3 active agents, anti-LAG-3 antibodies, a LAG-3-binding molecules, and a LAG-3 binding-fragments for use in the treatment of cancer, as well as corresponding methods, uses, pharmaceutical compositions, treatments, and production methods therein.

In certain embodiments, the anti-LAG-3 active agent comprises a LAG-3 binding-fragment of an anti-LAG-3 antibody, including (but not limited to) immunoconjugates, diabodies, BiTEs, bispecific antibodies, antibody-bonding fragments, and similar molecules.

In certain embodiments, the anti-LAG-3 antibody or fragment thereof comprises an Fc Region that is of the IgG1, IgG2, IgG3, IgG4, IgA1, or IgA2 isotype. In certain embodiments, the anti-LAG-3 antibody or fragment thereof comprises an Fc Region that is of the IgG1 isotype. In certain embodiments, the anti-LAG-3 antibody or fragment thereof comprises an Fc Region that is of the IgG1 isotype, wherein the amino acid sequence of the IgG1 heavy chain constant region comprises: an N297A mutation, numbered according to the EU numbering system; or an N297Q mutation, numbered according to the EU numbering system. In certain embodiments, the anti-LAG-3 antibody or fragment thereof comprises an Fe Region that is of the IgG4 isotype. In certain embodiments, the anti-LAG-3 antibody or fragment thereof comprises an Fe Region that is of the IgG4 isotype, wherein the amino acid sequence of the IgG4 heavy chain constant region comprises: an S228P mutation, numbered according to the EU numbering system; or an N297Q mutation, numbered according to the EU numbering system.

In certain embodiments, the anti-LAG-3 active agent is administered intravenously. In certain embodiments, the anti-LAG-3 active agent is administered intravenously once every two weeks, once every three weeks, or once every four weeks. In certain embodiments, the anti-LAG-3 active agent is administered intravenously once every two weeks. In certain embodiments, the anti-LAG-3 active agent is administered intravenously once every three weeks. In certain embodiments, the anti-LAG-3 active agent is administered intravenously once every four weeks.

### LAG-3 CDRs

In certain embodiments, the anti-LAG-3 active agent comprises an anti-LAG-3 antibody, or LAG-3 binding-fragment thereof which comprises: (i) a Heavy Chain Variable Domain (VH), comprising a CDRH1 Domain, a CDRH2 Domain, and a CDRH3 Domain; and (ii) a Light Chain Variable Domain (VL), comprising a CDRL1 Domain, a CDRL2 Domain, and a CDRL3 Domain.

In certain embodiments, the CDRH1 Domain has the amino acid sequence DTYIH (SEQ ID NO: 21).

In certain embodiments, the CDRH2 Domain has the amino acid sequence

(SEQ ID NO: 22)
EIDPANDNTKYDPKFQG.

In certain embodiments, the CDRH3 Domain has the amino acid sequence YYYX₁YX₂VGGFDY (SEQ ID NO: 55), wherein: X₁ is K or R; and X₂ is D or E. In certain embodiments, the CDRH3 Domain has the amino acid sequence YYYKYDVGGFDY (SEQ ID NO: 23).

In certain embodiments, the CDRL1 Domain has the amino acid sequence

(SEQ ID NO: 24)
SVSSSISSSNLH.

In certain embodiments, the CDRL2 Domain has the amino acid sequence GTSNLAS (SEQ ID NO: 25).

In certain embodiments, the CDRL3 Domain has the amino acid sequence

(SEQ ID NO: 26)
QQWSSYPFT.

### LAG-3 VHs and VLs

In certain embodiments, the Heavy Chain Variable Domain (VH) of the anti-LAG-3 antibody or fragment thereof comprises an amino acid sequence which is at least 75%, 80%, 85%, 90%, 95%, 99%, or 100% identical to:

(SEQ ID NO: 27)
QVQMVQSGAEVKKPGASVKVSCKASGFNIKDTYIHWVRQAPGQGLEWMG
EIDPANDNTKYDPKFQGRVTITADTSTSTVYMELSSLRSEDTAVYYCAT
YYYKYDVGGFDYWGQGTLVTVSS.

In certain embodiments, the Light Chain Variable Domain (VL) of the anti-LAG-3 antibody or fragment thereof comprises an amino acid sequence which is at least 75%, 80%, 85%, 90%, 95%, 99%, or 100% identical to:

(SEQ ID NO: 28)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIK.

### LAG-3 HC and LC

In certain embodiments, the anti-LAG-3 antibody or fragment thereof comprises a Heavy Chain (HC) sequence which is at least 75%, 80%, 85%, 90%, 95%, 99%, or 100% identical to:

(SEQ ID NO: 29)
QVQMVQSGAEVKKPGASVKVSCKASGFNIKDTYIHWVRQAPGQGLEWMG
EIDPANDNTKYDPKFQGRVTITADTSTSTVYMELSSLRSEDTAVYYCAT
YYYKYDVGGFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTK
PREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG.

In certain embodiments, the anti-LAG-3 antibody or fragment thereof comprises a Light Chain (LC) sequence which is at least 75%, 80%, 85%, 90%, 95%, 99%, or 100% identical to:

(SEQ ID NO: 30)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

### II. Administration and Treatments

### Pharmaceutical Compositions

In certain embodiments, active agents of the present disclosure are prepared as, or comprised in, pharmaceutical compositions. In certain embodiments, such compositions comprise one or more active agent of the present disclosure (e.g., anti-PD-1 active agent, anti-LAG-3 active agent, anti-TIM-3 active agent) and one or more therapeutically acceptable excipients (e.g., carrier, solvent, or delivery vehicle). In certain embodiments, pharmaceutical compositions of the present disclosure comprise a therapeutically effective amount of an anti-PD-1 active agent. In certain embodiments, pharmaceutical compositions of the present disclosure comprise a therapeutically effective amount of an anti-LAG-3 active agent. In certain embodiments, pharmaceutical compositions of the present disclosure comprise a therapeutically effective amount of an anti-TIM-3 active agent.

In certain embodiments, pharmaceutical compositions of the present disclosure are aqueous compositions (i.e., compositions which comprise water). In certain embodiments, pharmaceutical compositions of the present disclosure comprise water, sterilized or sanitized water, or Water-for-injection (WFI).

In certain embodiments, pharmaceutical compositions of the present disclosure comprise one or more of the following: pH buffered solutions (e.g., acetate buffers, phosphate buffered saline (PBS), HEPES, TES, MOPS), isotonic saline, Ringer's solution, polyols (e.g., glycerol, propylene glycol, liquid polyethylene glycol), alginic acid, ethyl alcohol, emulsifiers (e.g., polysorbates such as Polysorbate 80) and therapeutically acceptable mixtures thereof. In certain embodiments, pharmaceutical compositions of the present disclosure comprise phosphate buffered saline (PBS) or an acetate component (e.g., acetic acid, acetate salts, and/or an acetate buffer). In certain embodiments, pharmaceutical compositions of the present disclosure comprise thickening and solubilizing agents, such as glucose, polyethylene glycol, and polypropylene glycol and mixtures thereof.

In certain embodiments, pharmaceutical compositions of the present disclosure comprise a carrier. In certain embodiments, the carrier is one or more sterile liquids, such as water and oils. In certain embodiments, carriers are of petroleum, animal, vegetable, or synthetic origin, such as peanut oil, soybean oil, mineral oil, sesame oil, and similar oils. In certain embodiments, the pharmaceutical compositions comprise saline solutions, aqueous dextrose, and/or glycerol solutions (particularly for injectable solutions). Other suitable pharmaceutical carriers include, but are not limited to, starch, glucose, lactose, sucrose, trehalose, gelatin, malt, rice, flour, chalk, silica gel, sodium stearate, glycerol monostearate, talc, sodium chloride, dried skim milk, glycerol, propylene, glycol, sodium acetate, sorbitan monolaurate, triethanolamine oleate and cyclodextrins, water, ethanol, and the like. In certain embodiments, the pharmaceutical compositions comprise wetting or emulsifying agents, or pH buffering agents. In some embodiments, the emulsifying agent comprise polysorbates (e.g., Polysorbate 20, Polysorbate 40, Polysorbate 60 and Polysorbate 80).

In certain embodiments, pharmaceutical compositions of the present disclosure comprise an adjuvant. Non-limiting examples of appropriate adjuvants include, but are not limited to, Complete Freund's Adjuvant (CFA), Incomplete Freund's Adjuvant (IFA), the RIBI adjuvant System (RAS), TiterMax, muramyl peptides, Syntex Adjuvant Formulation (SAF), alum (aluminum hydroxide and/or aluminum phosphate), aluminum salt adjuvants, Gerbu Adjuvants (GERBU Biochemicals GmbH), nitrocellulose absorbed antigen, encapsulated or entrapped antigen, 3 De-O-acylated monophosphoryl lipid A (3 D-MPL), immunostimulatory oligonucleotides, toll-like receptor (TLR) ligands, mannan-binding lectin (MBL) ligands, stimulator of interferon genes (STING) agonists, immuno-stimulating complexes such as saponins, Quil A, QS-21, QS-7, ISCOMATRIX, and others. Other adjuvants include CpG oligonucleotides and double stranded RNA molecules, such as poly(A) and poly(U).

In certain embodiments, pharmaceutical compositions of the present disclosure comprise one or more pharmaceutical acceptable excipients. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: acetic acid, aluminum stearate, butylated hydroxytoluene (BHT), calcium carbonate, calcium chloride, calcium phosphate (dibasic), calcium stearate, carboxymethyl celluloses, croscarmellose, crosslinked polyvinyl pyrrolidone, citric acid, crospovidone, cysteine, ethylcellulose, gelatin, glucose, glucuronic acid, gluconic acid, hydroxypropyl cellulose, hydroxypropyl methylcellulose, hydroxybutanedioic acid, inosite, lactose, magnesium chloride, magnesium stearate, maltitol, mannitol, methionine, methylcellulose, methyl paraben, microcrystalline cellulose, phosphoric acid, polyethylene glycol, polyvinyl pyrrolidone, povidone, pregelatinized starch, propyl paraben, retinyl palmitate, saccharose, shellac, silicon dioxide, sodium acetate, sodium carbonate, sodium bicarbonate, sodium carboxymethyl cellulose, sodium chloride, sodium citrate, sodium hydroxide, sodium phosphate, sodium starch glycolate, sorbitol, starch (corn), stearic acid, sucrose, talc, titanium dioxide, vitamin A, vitamin E, vitamin C, xylitol, zinc stearate, and combinations thereof.

In certain embodiments, pharmaceutical compositions of the present disclosure comprise aqueous vehicles, nonaqueous vehicles, antimicrobial agents, isotonic agents, buffers, antioxidants, suspending and dispersing agents, emulsifying agents, sequestering or chelating agents, and other pharmaceutically acceptable substances. Examples of aqueous vehicles include Sodium Chloride Injection, Ringer's Injection, Isotonic Dextrose Injection, Sterile Water Injection, Dextrose and Lactated Ringer's Injection. Examples of nonaqueous vehicles include fixed oils of vegetable origin, cottonseed oil, corn oil, sesame oil, and peanut oil. Examples of antimicrobial agents include phenols or cresols, mercurials, benzyl alcohol, chlorobutanol, methyl and propyl p-hydroxybenzoic acid esters, thimerosal, benzalkonium chloride, and benzethonium chloride. Examples of isotonic agents include sodium chloride and dextrose. Examples of buffers include acetate, phosphate, citrate, and other organic acids. Examples of antioxidants include tocopherols (e.g., Vitamin E), sodium bisulfate, ascorbic acid, and methionine. Examples of suspending and dispersing agents include sodium carboxymethylcellulose, hydroxypropyl methylcellulose and polyvinylpyrrolidone. Examples of emulsifying agents include Polysorbate 80 (TWEEN® 80). Examples of sequestering or chelating agents include EDTA. Other examples of pharmaceutical vehicles also include ethyl alcohol, polyethylene glycol and propylene glycol for water miscible vehicles; and sodium hydroxide, hydrochloric acid, citric acid, acetic acid, or lactic acid for pH adjustment.

In certain embodiments, pharmaceutical compositions of the present disclosure comprise preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONIC™ or polyethylene glycol (PEG).

In certain embodiments, ingredients of compositions of the present disclosure are supplied either separately or mixed together in unit dosage form. In certain embodiments where a composition is to be administered by infusion, it can be dispensed with an infusion bottle containing sterile pharmaceutical grade water or saline. In certain embodiments where the composition is administered by injection, an ampoule of sterile water for injection or saline can be provided, and the ingredients may be mixed prior to administration.

Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. In certain embodiments, the pharmaceutical compositions take the form of solutions, suspensions, emulsion, powders, formulations for subcutaneous delivery, sustained-release compositions, and the like.

### Therapeutic Applications and Administration

In certain embodiments, active agents and compositions of the present disclosure are used as therapeutics for preventing and/or treating a disease in a subject (e.g., cancer). In certain embodiments, active agents and compositions of the present disclosure are used as therapeutics for regulating (e.g., negatively regulating or positively regulating) T-cell proliferation, function and homeostasis in a subject.

In certain embodiments, the present disclosure provides methods for treating a disease (e.g., cancer) in a human subject in need thereof. In certain embodiments, the present disclosure provides methods for regulating (e.g., negatively regulating or positively regulating) T-cell proliferation, function, and homeostasis in a human subject in need thereof. In certain embodiments, the method comprises administering to the subject: (i) a therapeutically effective amount of an anti-PD-1 active agent (e.g., retifanlimab); (ii) a therapeutically effective amount of an anti-TIM-3 active agent (e.g., Antibody A); and (iii) a therapeutically effective amount of an anti-LAG-3 active agent (e.g., Antibody B). In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; (ii) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent; and (iii) from about 300 mg to about 800 mg, in some embodiments about 350 mg to about 750 mg, and in some embodiments about 350 mg, about 450 mg or about 750 mg, of an anti-LAG-3 active agent. In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent once every three to five weeks, and in some embodiments once every three weeks or once every four weeks; (ii) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent once every one to four weeks, and in some embodiments once every two weeks, once every three weeks, or once every four weeks; and (iii) from about 300 mg to about 800 mg, in some embodiments about 350 mg to about 750 mg, and in some embodiments about 350 mg, about 450 mg or about 750 mg of an anti-LAG-3 active agent once every one to four weeks, and in some embodiments once every two weeks, once every three weeks, or once every four weeks.

In certain embodiments, the method comprises administering to the subject: (i) about 500 mg of an anti-PD-1 active agent (e.g., retifanlimab); (ii) about 400 mg of an anti-TIM-3 active agent (e.g., Antibody A); and (iii) about 350 mg of an anti-LAG-3 active agent (e.g., Antibody B). In certain embodiments, the method comprises administering to the subject: (i) about 375 mg of an anti-PD-1 active agent (e.g., retifanlimab); (ii) about 500 mg of an anti-TIM-3 active agent (e.g., Antibody A); and (iii) about 450 mg of an anti-LAG-3 active agent (e.g., Antibody B). In certain embodiments, the method comprises administering to the subject: (i) about 375 mg of an anti-PD-1 active agent (e.g., retifanlimab); (ii) about 1000 mg of an anti-TIM-3 active agent (e.g., Antibody A); and (iii) about 750 mg of an anti-LAG-3 active agent (e.g., Antibody B). In certain embodiments, the method comprises administering to the subject: (i) about 500 mg of an anti-PD-1 active agent (e.g., retifanlimab) once every four weeks; (ii) about 400 mg of an anti-TIM-3 active agent (e.g., Antibody A) once every two weeks; and (iii) about 350 mg of an anti-LAG-3 active agent (e.g., Antibody B) once every two weeks. In certain embodiments, the method comprises administering to the subject: (i) about 375 mg of an anti-PD-1 active agent (e.g., retifanlimab) once every three weeks; (ii) about 500 mg of an anti-TIM-3 active agent (e.g., Antibody A) once every three weeks; and (iii) about 450 mg of an anti-LAG-3 active agent (e.g., Antibody B) once every three weeks. In certain embodiments, the method comprises administering to the subject: (i) about 375 mg of an anti-PD-1 active agent (e.g., retifanlimab) once every three weeks; (ii) about 1000 mg of an anti-TIM-3 active agent (e.g., Antibody A) once every three weeks; and (iii) about 750 mg of an anti-LAG-3 active agent (e.g., Antibody B) once every three weeks.

In certain embodiments, the method comprises administering to the subject: (i) a therapeutically effective amount of an anti-PD-1 active agent (e.g., retifanlimab); and (ii) a therapeutically effective amount of an anti-TIM-3 active agent (e.g., Antibody A). In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; and (ii) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent. In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent once every three to five weeks, and in some embodiments once every three weeks or once every four weeks; and (ii) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent once every one to four weeks, and in some embodiments once every two weeks, once every three weeks, or once every four weeks. In certain embodiments, the method comprises administering to the subject: (i) about 500 mg of an anti-PD-1 active agent (e.g., retifanlimab) once every four weeks; and (ii) about 400 mg of an anti-TIM-3 active agent (e.g., Antibody A) once every two weeks. In certain embodiments, the method comprises administering to the subject: (i) about 375 mg of an anti-PD-1 active agent (e.g., retifanlimab) once every three weeks; and (ii) about 500 mg of an anti-TIM-3 active agent (e.g., Antibody A) once every three weeks. In certain embodiments, the method comprises administering to the subject: (i) about 375 mg of an anti-PD-1 active agent (e.g., retifanlimab) once every three weeks; and (ii) about 1000 mg of an anti-TIM-3 active agent (e, Antibody A) once every three weeks.

In certain embodiments, the method comprises administering to the subject: (i) a therapeutically effective amount of an anti-PD-1 active agent (e.g., retifanlimab); and (ii) a therapeutically effective amount of an anti-LAG-3 active agent (e.g., Antibody B). In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; and (ii) from about 300 mg to about 800 mg, in some embodiments about 350 mg to about 750 mg, and in some embodiments about 350 mg, about 450 mg or about 750 mg, of an anti-LAG-3 active agent. In certain embodiments, the method comprises administering to the subject: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent once every three to five weeks, and in some embodiments once every three weeks or once every four weeks; and (ii) from about 300 to about 800 mg, in some embodiments about 350 mg to about 750 mg, and in some embodiments about 350 mg, about 450 mg or about 750 mg of an anti-LAG-3 active agent once every one to four weeks, and in some embodiments once every two weeks, once every three weeks, or once every four weeks. In certain embodiments, the method comprises administering to the subject: (i) about 500 mg of an anti-PD-1 active agent (e.g., retifanlimab) once every four weeks; and (ii) about 400 mg of an anti-LAG-3 active agent (e.g., Antibody B) once every two weeks. In certain embodiments, the method comprises administering to the subject: (i) about 375 mg of an anti-PD-1 active agent (e.g., retifanlimab) once every three weeks; and (ii) about 450 mg of an anti-LAG-3 active agent (e.g., Antibody B) once every three weeks. In certain embodiments, the method comprises administering to the subject: (i) about 375 mg of an anti-PD-1 active agent (e.g., retifanlimab) once every three weeks; and (ii) about 750 mg of an anti-LAG-3 active agent (e.g., Antibody B) once every three weeks.

In certain embodiments, the method comprises administering to the subject: (i) a therapeutically effective amount of an anti-TIM-3 active agent (e.g., Antibody A); and (ii) a therapeutically effective amount of an anti-LAG-3 active agent (e.g., Antibody B). In certain embodiments, the method comprises administering to the subject: (i) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent; and (ii) from about 300 mg to about 800 mg, in some embodiments about 350 mg to about 750 mg, and in some embodiments about 350 mg, about 450 mg or about 750 mg, of an anti-LAG-3 active agent. In certain embodiments, the method comprises administering to the subject: (i) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent every one to four weeks, and in some embodiments once every two weeks, once every three weeks, or once every four weeks; and (ii) from about 300 mg to about 800 mg, in some embodiments about 350 mg to about 750 mg, and in some embodiments about 350 mg, about 450 mg or about 750 mg of an anti-LAG-3 active agent once every one to four weeks, and in some embodiments once every two weeks, once every three weeks, or once every four weeks. In certain embodiments, the method comprises administering to the subject: (i) about 400 mg of an anti-TIM-3 active agent (e.g., Antibody A) once every two weeks; and (ii) about 350 mg of an anti-LAG-3 active agent (e.g., Antibody B) once every two weeks. In certain embodiments, the method comprises administering to the subject: (i) about 500 mg of an anti-TIM-3 active agent (e.g., Antibody A) once every three weeks; and (ii) about 450 mg of an anti-LAG-3 active agent (e.g., Antibody B) once every three weeks. In certain embodiments, the method comprises administering to the subject: (i) about 1000 mg of an anti-TIM-3 active agent (e.g., Antibody A) once every three weeks; and (ii) about 750 mg of an anti-LAG-3 active agent (e.g., Antibody B) once every three weeks.

In certain embodiments, the present disclosure provides a combination therapy comprising: a therapeutically effective amount of an anti-PD-1 active agent (e.g., from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg), a therapeutically effective amount of an anti-TIM-3 active agent (e.g., from about 300 mg to about 1000 mg, in some embodiments about 400 mg to about 1000 mg, in some embodiments about 350 mg to about 500 mg, and in some embodiments about 400 mg, about 500 mg or about 1000 mg), and a therapeutically effective amount of an anti-LAG-3 active agent (e.g., from about 300 mg to about 800 mg, in some embodiments about 350 mg to about 750 mg, and in some embodiments about 350 mg, about 450 mg or about 750 mg).

In certain embodiments, the combination therapy comprises: (i) about 500 mg of an anti-PD-1 active agent (e.g., retifanlimab); (ii) about 400 mg of an anti-TIM-3 active agent (e.g., Antibody A); and (iii) about 350 mg of an anti-LAG-3 active agent (e.g., Antibody B). In certain embodiments, the combination therapy comprises: (i) about 375 mg of an anti-PD-1 active agent (e.g., retifanlimab); (ii) about 500 mg of an anti-TIM-3 active agent (e.g., Antibody A); and (iii) about 450 mg of an anti-LAG-3 active agent (e.g., Antibody B). In certain embodiments, the combination therapy comprises: (i) about 375 mg of an anti-PD-1 active agent (e.g., retifanlimab); (ii) about 1000 mg of an anti-TIM-3 active agent (e.g., Antibody A); and (iii) about 750 mg of an anti-LAG-3 active agent (e.g., Antibody B). In certain embodiments, the combination therapy comprises: (i) about 500 mg of an anti-PD-1 active agent (e.g., retifanlimab) formulated for administration once every four weeks; (ii) about 400 mg of an anti-TIM-3 active agent (e.g., Antibody A) formulated for administration once every two weeks; and (iii) about 350 mg of an anti-LAG-3 active agent (e.g., Antibody B) formulated for administration once every two weeks. In certain embodiments, the combination therapy comprises: (i) about 375 mg of an anti-PD-1 active agent (e.g., retifanlimab) formulated for administration once every three weeks; (ii) about 500 mg of an anti-TIM-3 active agent (e.g., Antibody A) formulated for administration once every three weeks; and (iii) about 450 mg of an anti-LAG-3 active agent (e.g., Antibody B) formulated for administration once every three weeks. In certain embodiments, the combination therapy comprises: (i) about 375 mg of an anti-PD-1 active agent (e.g., retifanlimab) formulated for administration once every three weeks; (ii) about 1000 mg of an anti-TIM-3 active agent (e.g., Antibody A) formulated for administration once every three weeks; and (iii) about 750 mg of an anti-LAG-3 active agent (e, Antibody B) formulated for administration once every three weeks.

In certain embodiments, the combination therapy comprises: (i) a therapeutically effective amount of an anti-PD-1 active agent (e.g., retifanlimab); and (ii) a therapeutically effective amount of an anti-TIM-3 active agent (e.g., Antibody A). In certain embodiments, the combination therapy comprises: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; and (ii) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent. In certain embodiments, the combination therapy comprises: (i) about 500 mg of an anti-PD-1 active agent (e.g., retifanlimab); and (ii) about 400 mg of an anti-TIM-3 active agent (e.g., Antibody A). In certain embodiments, the combination therapy comprises: (i) about 375 mg of an anti-PD-1 active agent (e.g., retifanlimab); and (ii) about 500 mg of an anti-TIM-3 active agent (e.g., Antibody A). In certain embodiments, the combination therapy comprises: (i) about 375 mg of an anti-PD-1 active agent (e.g., retifanlimab); and (ii) about 1000 mg of an anti-TIM-3 active agent (e.g., Antibody A).

In certain embodiments, the combination therapy comprises: (i) a therapeutically effective amount of an anti-PD-1 active agent (e.g., retifanlimab); and (ii) a therapeutically effective amount of an anti-LAG-3 active agent (e.g., Antibody B). In certain embodiments, the combination therapy comprises: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg, of an anti-PD-1 active agent; and (ii) from about 300 mg to about 800 mg, in some embodiments about 350 mg to about 750 mg, and in some embodiments about 350 mg, about 450 mg or about 750 mg, of an anti-LAG-3 active agent. In certain embodiments, the combination therapy comprises: (i) about 500 mg of an anti-PD-1 active agent (e.g., retifanlimab); and (ii) about 400 mg of an anti-LAG-3 active agent (e.g., Antibody B). In certain embodiments, the combination therapy comprises: (i) about 375 mg of an anti-PD-1 active agent (e.g., retifanlimab); and (ii) about 450 mg of an anti-LAG-3 active agent (e.g., Antibody B). In certain embodiments, the combination therapy comprises: (i) about 375 mg of an anti-PD-1 active agent (e.g., retifanlimab); and (ii) about 750 mg of an anti-LAG-3 active agent (e.g., Antibody B).

In certain embodiments, the combination therapy comprises: (i) a therapeutically effective amount of an anti-TIM-3 active agent (e.g., Antibody A); and (ii) a therapeutically effective amount of an anti-LAG-3 active agent (e.g., Antibody B). In certain embodiments, the combination therapy comprises: (i) from about 300 mg to about 1000 mg, in some embodiments about 400 mg to about 1000 mg, in some embodiments about 350 mg to about 500 mg, and in some embodiments about 400 mg, about 500 mg or about 1000 mg, of an anti-TIM-3 active agent; and (ii) from about 300 mg to about 800 mg, in some embodiments about 350 mg to about 750 mg, and in some embodiments about 350 mg, about 450 mg or about 750 mg, of an anti-LAG-3 active agent. In certain embodiments, the combination therapy comprises: (i) about 400 mg of an anti-TIM-3 active agent (e.g., Antibody A); and (ii) about 350 mg of an anti-LAG-3 active agent (e.g., Antibody B). In certain embodiments, the combination therapy comprises: (i) about 500 mg of an anti-TIM-3 active agent (e.g., Antibody A); and (ii) about 450 mg of an anti-LAG-3 active agent (e.g., Antibody B). In certain embodiments, the combination therapy comprises: (i) about 1000 mg of an anti-TIM-3 active agent (e.g., Antibody A); and (ii) about 750 mg of an anti-LAG-3 active agent (e.g., Antibody B).

In certain embodiments, the combination therapy is for use in the treatment of cancer in a human subject in need thereof. In certain embodiments, combination therapies of the present disclosure are for use in a method for treating a disease in a human subject in need thereof (e.g., cancer). In certain embodiments, combination therapies of the present disclosure are for use in regulating (e.g., negatively regulating or positively regulating) T-cell proliferation, function, and homeostasis in a human subject in need thereof. In certain embodiments, combination therapies of the present disclosure are for use in the manufacturing/production of a medicament for use in treating a disease in a human subject in need thereof (e.g., cancer). In certain embodiments, combination therapies of the present disclosure are for use in the manufacturing/production of a medicament for use in regulating (e.g., negatively regulating or positively regulating) T-cell proliferation, function, and homeostasis in a human subject in need thereof.

In certain embodiments, the present disclosure provides a kit for treating a disease (e.g., cancer) in a human subject in need thereof. In certain embodiments, the present disclosure provides a kit for regulating (e.g., negatively regulating or positively regulating) T-cell proliferation, function, and homeostasis in a human subject in need thereof. In certain embodiments, the kit comprises: from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent; from about 300 to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent; and from about 300 to about 800 mg, in some embodiments about 350 mg to about 750 mg, and in some embodiments about 350 mg, about 450 mg or about 750 mg of an anti-LAG-3 active agent. In certain embodiments, the kit comprises instructions to administer the anti-PD-1 active agent (e.g., once every three weeks or once every four weeks) and to administer the anti-TIM-3 and anti-LAG-3 active agents (e.g., once every two weeks, once every three weeks, or once every four weeks). In certain embodiments, the kit comprises: from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent; and from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent; and from about 300 mg to about 800 mg, in some embodiments about 350 mg to about 750 mg, and in some embodiments about 350 mg, about 450 mg or about 750 mg of an anti-LAG-3 active agent. In certain embodiments, the kit comprises instructions to administer the anti-PD-1 active agent (e.g., once every three weeks or once every four weeks) and to administer the anti-TIM-3 and anti-LAG-3 active agents (e.g., once every two weeks, once every three weeks, or once every four weeks).

In certain embodiments, the kit comprises: (i) about 500 mg of an anti-PD-1 active agent (e.g., retifanlimab); (ii) about 400 mg of an anti-TIM-3 active agent (e.g., Antibody A); and (iii) about 350 mg of an anti-LAG-3 active agent (e.g., Antibody B); and also comprises instructions to administer the anti-PD-1 active agent once every four weeks, and to administer the anti-TIM-3 and anti-LAG-3 active agents once every two weeks.

In certain embodiments, the kit comprises: (i) about 375 mg of an anti-PD-1 active agent (e.g., retifanlimab); (ii) about 500 mg of an anti-TIM-3 active agent (e.g., Antibody A); and (iii) about 450 mg of an anti-LAG-3 active agent (e.g., Antibody B); and also comprises instructions to administer the anti-PD-1 active agent, the anti-TIM-3 active agent, and the anti-LAG-3 active agent once every three weeks.

In certain embodiments, the kit comprises: (i) about 375 mg of an anti-PD-1 active agent (e.g., retifanlimab); (ii) about 1000 mg of an anti-TIM-3 active agent (e.g., Antibody A); and (iii) about 750 mg of an anti-LAG-3 active agent (e.g., Antibody B); and also comprises instructions to administer the anti-PD-1 active agent, the anti-TIM-3 active agent, and the anti-LAG-3 active agent once every three weeks.

In certain embodiments, the kit comprises: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent; and (ii) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent. In certain embodiments, the kit comprises instructions to administer the anti-PD-1 active agent (e.g., once every three weeks or once every four weeks) and to administer the anti-TIM-3 active agent (e.g., once every two weeks, once every three weeks, or once every four weeks). In certain embodiments, the kit comprises: (i) about 500 mg of an anti-PD-1 active agent (e.g., retifanlimab); and (ii) about 400 mg of an anti-TIM-3 active agent (e.g., Antibody A); and also comprises instructions to administer the anti-PD-1 active agent once every four weeks, and to administer the anti-TIM-3 active agent once every two weeks. In certain embodiments, the kit comprises: (i) about 375 mg of an anti-PD-1 active agent (e.g., retifanlimab); and (ii) about 500 mg of an anti-TIM-3 active agent (e.g., Antibody A); and also comprises instructions to administer the anti-PD-1 active agent and the anti-TIM-3 active agent once every three weeks. In certain embodiments, the kit comprises: (i) about 375 mg of an anti-PD-1 active agent (e.g., retifanlimab); and (ii) about 1000 mg of an anti-TIM-3 active agent (e.g., Antibody A); and also comprises instructions to administer the anti-PD-1 active agent and the anti-TIM-3 active agent once every three weeks.

In certain embodiments, the kit comprises: (i) from about 300 mg to about 600 mg, in some embodiments about 375 mg to about 550 mg, in some embodiments about 375 mg to about 500 mg, in some embodiments about 450 mg to about 550 mg, and in some embodiments about 375 mg or about 500 mg of an anti-PD-1 active agent; and (ii) from about 300 mg to about 800 mg, in some embodiments about 350 mg to about 750 mg, and in some embodiments about 350 mg, about 450 mg or about 750 mg of an anti-LAG-3 active agent. In certain embodiments, the kit comprises instructions to administer the anti-PD-1 active agent (e.g., once every three weeks or once every four weeks) and to administer the anti-LAG-3 active agent (e.g., once every two weeks, once every three weeks, or once every four weeks). In certain embodiments, the kit comprises: (i) about 500 mg of an anti-PD-1 active agent (e.g., retifanlimab); and (ii) about 350 mg of an anti-LAG-3 active agent (e.g., Antibody B); and also comprises instructions to administer the anti-PD-1 active agent once every four weeks, and to administer the anti-LAG-3 active agent once every two weeks. In certain embodiments, the kit comprises: (i) about 375 mg of an anti-PD-1 active agent (e.g., retifanlimab); and (ii) about 450 mg of an anti-LAG-3 active agent (e.g., Antibody B); and also comprises instructions to administer the anti-PD-1 active agent and the anti-LAG-3 active agent once every three weeks. In certain embodiments, the kit comprises: (i) about 375 mg of an anti-PD-1 active agent (e.g., retifanlimab); and (ii) about 750 mg of an anti-LAG-3 active agent (e.g., Antibody B); and also comprises instructions to administer the anti-PD-1 active agent and the anti-LAG-3 active agent once every three weeks.

In certain embodiments, the kit comprises: (i) from about 300 mg to about 1000 mg, in some embodiments about 400 mg to about 1000 mg, in some embodiments about 350 mg to about 500 mg, and in some embodiments about 400 mg, about 500 mg or about 1000 mg of an anti-TIM-3 active agent; and (ii) from about 300 mg to about 800 mg, in some embodiments about 350 mg to about 750 mg, and in some embodiments about 350 mg, about 450 mg or about 750 mg of an anti-LAG-3 active agent. In certain embodiments, the kit comprises instructions to administer the anti-TIM-3 and anti-LAG-3 active agents (e.g., once every two weeks, once every three weeks, or once every four weeks). In certain embodiments, the kit comprises: (i) about 400 mg of an anti-TIM-3 active agent (e.g., Antibody A); and (ii) about 350 mg of an anti-LAG-3 active agent (e.g., Antibody B); and also comprises instructions to administer the anti-TIM-3 and anti-LAG-3 active agents once every two weeks. In certain embodiments, the kit comprises: (i) about 500 mg of an anti-TIM-3 active agent (e.g., Antibody A); and (ii) about 450 mg of an anti-LAG-3 active agent (e.g., Antibody B); and also comprises instructions to administer the anti-TIM-3 active agent and the anti-LAG-3 active agent once every three weeks. In certain embodiments, the kit comprises: (i) about 1000 mg of an anti-TIM-3 active agent (e.g., Antibody A); and (ii) about 750 mg of an anti-LAG-3 active agent (e.g., Antibody B); and also comprises instructions to administer the anti-TIM-3 active agent and the anti-LAG-3 active agent once every three weeks.

In certain embodiments, the combination therapies, methods, and kits of the present disclosure are used in the treatment of cancer. Examples of cancers that may be treated include, but are not limited to: an adrenal gland tumor, AIDS-associated cancer, alveolar soft part sarcoma, anal cancer, squamous cell carcinoma of an anal canal (SCAC), an astrocytic tumor, bladder cancer, bone cancer, brain and spinal cord cancer, a metastatic brain tumor, breast cancer, HER2+ breast cancer, Triple-Negative Breast Cancer (TNBC), a carotid body tumors, cervical cancer, HPV-related cervical cancer, chondrosarcoma, chordoma, chromophobe renal cell carcinoma, clear cell carcinoma, colon cancer, colorectal cancer, cutaneous benign fibrous histiocytoma, a desmoplastic small round cell tumor, an ependymoma, endometrial cancer, metastatic endometrial cancer, unselected endometrial cancer, MSI-high endometrial cancer, dMMR endometrial cancer, DNA polymerase F (POLE) exonuclease domain mutation positive endometrial cancer, a Ewing's tumor, Ewing's sarcoma, extraskeletal myxoid chondrosarcoma, a fibrogenesis imperfecta ossium, a fibrous dysplasia of the bone, gallbladder or bile duct cancer, cholangiocarcinoma bile duct cancer, gastric cancer, gastroesophageal junction (GEJ) cancer, a gestational trophoblastic disease, a germ cell tumor, glioma, glioblastoma, head and neck cancer, squamous cell carcinoma of head and neck (SCCHN) including recurrent or metastatic PD-L1+ SCCHN, a hematological malignancy, hepatocellular carcinoma, an islet cell tumor, a Kaposi's Sarcoma, kidney cancer, renal cell carcinomas (RCC), clear cell RRC, papillary RCC and chromophobe RCC, leukemia, acute myeloid leukemia, a lipoma/benign lipomatous tumor, a liposarcoma/malignant lipomatous tumor, liver cancer, hepatocellular carcinoma liver cancer (HCC), lymphoma, diffuse large B-cell lymphoma (DLBCL), non-Hodgkin lymphoma (NHL), lung cancer, small cell lung cancer (SCLC), non-small cell lung cancer (NSCLC), medulloblastoma, melanoma (unresectable and/or metastatic), uveal melanoma, meningioma, mesothelioma, mesothelial pharyngeal cancer, multiple endocrine neoplasia, multiple myeloma, a myelodysplastic syndrome, a neuroblastoma, a neuroendocrine tumors, ovarian cancer, pancreatic cancer, papillary thyroid carcinoma, a parathyroid tumor, pediatric cancer, a peripheral nerve sheath tumor, pharyngeal cancer, a phaeochromocytoma, a pituitary tumor, prostate cancer, metastatic castration resistant prostate cancer (mCRPC), posterious uveal melanoma, a rare hematologic disorder, renal metastatic cancer, a rhabdoid tumor, a rhabdomyosarcoma, a sarcoma, skin cancer, Merkel cell carcinoma, a small round blue cell tumor of childhood, neuroblastoma, rhabdomyosarcoma, a soft-tissue sarcoma, squamous cell cancer, stomach cancer, a synovial sarcoma, testicular cancer, a thymic carcinoma, a thymoma, thyroid metastatic cancer, urothelial cancer, and uterine cancer.

In certain embodiments, the combination therapies, methods, and kits of the present disclosure are used in the treatment of melanoma (unresectable and/or metastatic), colorectal cancer, hepatocellular carcinoma, glioma, kidney cancer, breast cancer, multiple myeloma, bladder cancer, neuroblastoma; sarcoma, non-Hodgkin lymphoma, non-small cell lung cancer, ovarian cancer, pancreatic cancer, and rectal cancer. In certain embodiments, the combination therapies, methods, and kits of the present disclosure are used in the treatment of melanoma (unresectable and/or metastatic). In certain embodiments, the cancer is melanoma. In certain embodiments, the cancer is unresectable and/or metastatic melanoma.

In certain embodiments, the cancer being treated is characterized by a solid tumor. In certain embodiments, the cancer being treated is characterized by a metastatic lesion. In one embodiment, the cancer is a solid tumor. Examples of solid tumors include malignancies (e.g., sarcomas and carcinomas) of various organs, such as those affecting the lung, breast, ovarian, lymphoid, gastrointestinal (e.g., colon), genito-urinary tract (e.g., renal, urothelial, bladder cells, prostate), pharynx, CNS (e.g., brain, neural or glial cells), head and neck, skin (e.g., melanoma), pancreas, colon, rectal, renal-cell carcinoma, liver, small intestine, and esophagus. In certain embodiments, the cancer is an early, intermediate, late stage or metastatic cancer. In certain embodiments, the cancer is associated with elevated PD-1 activity (e.g., elevated PD-1 expression).

In certain embodiments, the cancer is a hematological cancer, for example, a leukemia, a lymphoma, or a myeloma. In certain embodiments, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In certain embodiments, the cancer is a lymphoma, for example, B-cell lymphoma, diffuse large B-cell lymphoma (DLBCL), activated B-cell like (ABC) diffuse large B-cell lymphoma, germinal center B-cell (GCB) diffuse large B-cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma. In certain embodiments the cancer is a myeloma, for example, multiple myeloma.

In certain embodiments, the cancer is a melanoma, e.g., an advanced melanoma. In certain embodiments, the cancer is an advanced or unresectable melanoma that does not respond to other therapies. In certain embodiments, the cancer is a melanoma with a BRAF mutation (e.g., a BRAF V600 mutation).

In certain embodiments, the cancer is squamous cell carcinoma of the head and neck (SCCHN). In certain embodiments, the cancer is recurrent or metastatic squamous cell carcinoma of the head and neck (SCCHN), including recurrent or metastatic PD-L1+ SCCHN.

In certain embodiments, the cancer comprises a tumor. In certain embodiments, the cancer comprises a locally advanced tumor, a metastatic solid tumor, or a combination thereof. In certain embodiments, the cancer comprises a tumor for which a PD-1 inhibitor is indicated.

In certain embodiments, the subject has received prior treatment with at least one anti-PD-1/anti-PDL-1 therapy. In certain embodiments, the subject has a cancer which failed a prior PD-1/PDL-1 inhibitor therapy. In certain embodiments, the subject has a cancer which continued to progress during a prior PD-1/PDL-1 inhibitor therapy. In certain embodiments, the cancer comprises a tumor that has acquired resistance to anti-PD-1 therapy. In certain embodiments, the cancer comprises a tumor that has innate resistance to anti-PD-1 therapy. In certain embodiments, the cancer comprises a tumor that has acquired resistance to anti-PD-1 therapy and an innate resistance to anti-PD-1 therapy. In certain embodiments, the subject is treatment-naive to anti-PDL-1 therapy. In certain embodiments, the tumor has a minimum LAG-3 expression of greater than or equal to 5% of LAG-3-positive immune cells (e.g., lymphocytes and macrophages) relative to all nucleated cells within the tumor region as shown by an appropriate immunohistochemical assay. The tumor region includes tumor cells, intratumoral stroma, and peritumoral stroma, with normal and/or adjacent uninvolved tissues not included. Suitable LAG-3 immunohistochemical assays are found in Wojcik et al., Consistent Measurement of LAG-3 expression Across Multiple Staining Platforms with the 17B4 Antibody Clone, bioRxiv (Feb. 22, 2022), doi.org/10.1101/2022.02.21.481075; and Johnson et al., Development of a LAG-3 Immunohistochemistry Assay for Melanoma, bioRxiv (Feb. 26, 2022), doi.org/10.1101/2022.02.25.481964.

In certain embodiments, the administration or treatment of the present disclosure produces at least one therapeutic effect. In certain embodiments, the therapeutic effect is a reduction in size of a tumor. In certain embodiments, the therapeutic effect is a reduction in number of metastasic lesions over time. In certain embodiments, the therapeutic effect is a complete response (e.g., disappearance of all target lesions according to RECIST v1.1 criteria). In certain embodiments, the therapeutic effect is a partial response (e.g., ≥30% decrease in sum of target lesion diameters, according to RECIST v1.1 criteria). In certain embodiments, the therapeutic effect is a stable disease (e.g., ≤30% decrease and ≤20% increase in sum of target lesion diameters, according to RECIST v1.1 criteria). In certain embodiments, the therapeutic effect is a progressive disease (e.g., ≥20% increase in sum of target lesion diameters, according to RECIST v1.1 criteria). In certain embodiments, the therapeutic effect is evaluated according to Response Evaluation Criteria in Solid Tumors (RECIST) criteria. In certain embodiments, the therapeutic effect is evaluated according to RECIST v1.1 criteria.

In certain embodiments, the combination therapies, methods, and kits of the present disclosure are used in combination with other therapeutic agents (e.g., anti-cancer agents), including other molecules (e.g., antibodies) that bind a target antigen (e.g., cancer-related antigen). In certain embodiments, the additional therapeutic agent is a chemotherapeutic, a radiotherapeutic, or a checkpoint targeting agent. In certain embodiments, the chemotherapeutic agent is a hypomethylating agent (e.g., azacitidine). In certain embodiments, the checkpoint targeting agent is selected from anti-CTLA-4 antibody, an antagonist anti-PDL-1 antibody, an antagonist anti-PDL-2 antibody, an antagonist anti-PD-1 antibody, an antagonist anti-TIM-3 antibody, an antagonist anti-LAG-3 antibody, an antagonist anti-CEACAM1 antibody, an agonist anti-CD137 antibody, an antagonist anti-CD73 antibody, an antagonist anti-CD96 antibody, an antagonist anti-TIGIT antibody, an antagonist anti-VISTA antibody, an agonist anti-GITR antibody, and an agonist anti-OX40 antibody. In certain embodiments, the additional therapeutic agent is a molecule that bind 5T4, B7H3, CD19, CD20, CD51, CD123, DR5, EGFR, EpCam, GD2, gpA33, HER2, ROR-1, TAG-72, VEGF-A antibody, and/or VEGFR2.

In certain embodiments, the combination therapies, methods, and kits of the present disclosure are used in combination with one or more immunogenic agent (e.g., tumor vaccines), for example those targeting MUC1, HPV, CEA, and transcription factors (e.g., Twist and brachyury). Examples of tumor vaccines include purified tumor antigens (including recombinant proteins, peptides, and carbohydrate molecules), autologous tumor cells, and/or allogeneic tumor cells.

In certain embodiments, the combination therapies, methods, and kits of the present disclosure are used in combination with one or more immunostimulatory agent (e.g., antibodies which activate host immune responsiveness to provide for increased levels of T-cell activation). Examples of immunostimulatory agent include anti-PD-1 antibodies, anti-PDL-1 antibodies, and/or an anti-CTLA4 antibodies, antibodies to molecules on the surface of dendritic cells that activate dendritic cell (DC) function and antigen presentation, anti-CD40 antibodies able to substitute for T-cell helper activity, activating antibodies to T-cell costimulatory molecules (e.g., PDL-1, CTLA-4, OX-40 4-1BB, ICOS), and/or stimulatory Chimeric Antigen Receptors (CARs).

In certain embodiments, the combination therapies, methods, and kits of the present disclosure are used in combination with one or more compound that targets an immunomodulatory enzyme such as IDO (indoleamine-(2,3)-dioxygenase) and/or TDO (tryptophan 2,3-dioxygenase). Examples of compounds that target an immunomodulatory enzyme (such as IDO and/or TDO) include epacadostat (Incyte Corp), F001287 (Flexus Biosciences/BMS), indoximod (NewLink Genetics), and NLG919 (NewLink Genetics).

In certain embodiments, the combination therapies, methods, and kits of the present disclosure are used in combination with one or more antibody-cytokine fusion proteins, also known as “immunocytokines” (see, e.g., Runbeck et al., Immunocytokines for Cancer, Antibodies 10(10), 2021). Examples of immunocytokines include immunocytokines containing lymphotoxin, interferons, TNF, TGFβRII, granulocyte-macrophage colony-stimulating factor (GM-CSF), IL-2, IL-3, IL-4, IL-6, IL-7, IL-8, IL-12, or NHS-IL12, IL-13, IL-15, or IL-15 superagonists, IL-17, CXCR1/2 inhibitor, granulocyte colony-stimulating factor (G-CSF), IL-10, vascular endothelial growth factor (VEGF), C-C motif chemokine ligand 21 (CCL21), and 4-1BBL.

In certain embodiments, the combination therapies, methods, and kits of the present disclosure are delivered using a delivery system known in the art. Examples of delivery systems include encapsulation in liposomes, microparticles, microcapsules, recombinant cells capable of expressing the active agent (e.g., antibody or antibody fragments), and/or receptor-mediated endocytosis.

In certain embodiments, the active agents, combination therapies, or compositions of the present disclosure are administered to the subject using one or more administration methods. Methods of administration for the present disclosure include, but are not limited to, parenteral administration (e.g., intradermal, intramuscular, intraperitoneal, intravenous, and subcutaneous), epidural, and mucosal (e.g., intranasal and oral routes). In certain embodiments, the active agents, combination therapies, or compositions are administered intramuscularly, intravenously, or subcutaneously. In certain embodiments, the active agents, combination therapies, or compositions are administered by infusion, by bolus injection, or by absorption through epithelial or mucocutaneous linings (e.g., oral mucosa and/or intestinal mucosa). In certain embodiments, the active agents, combination therapies, or compositions are administered together with other biologically active agents. Administration can be systemic or local.

In certain embodiments, a subject is administered a therapeutically effective amount of an active agent, combination therapy, or composition of the present disclosure. The effective amount can be in one administration, or in more than one administration. In some embodiments, the therapeutically effective amounts of the active agents, combination therapies, or compositions of the present disclosure are administered simultaneously or sequentially.

In certain embodiments, the anti-PD-1 active agent is administered concurrently with the anti-TIM-3 active agent. In certain embodiments, the anti-PD-1 active agent is administered concurrently with the anti-LAG-3 active agent. In certain embodiments, the anti-PD-1 active agent is administered concurrently with the anti-TIM-3 active agent and the anti-LAG-3 active agent. In certain embodiments, the anti-PD-1 active agent is administered before the anti-TIM-3 active agent. In certain embodiments, the anti-PD-1 active agent is administered before the anti-LAG-3 active agent. In certain embodiments, the anti-PD-1 active agent is administered before the anti-TIM-3 active agent and the anti-LAG-3 active agent. In certain embodiments, the anti-PD-1 active agent is administered after the anti-TIM-3 active agent. In certain embodiments, the anti-PD-1 active agent is administered after the anti-LAG-3 active agent. In certain embodiments, the anti-PD-1 active agent is administered after the anti-TIM-3 active agent and the anti-LAG-3 active agent. In certain embodiments, the anti-PD-1 active agent is administered before the anti-TIM-3 active agent and after the anti-LAG-3 active agent. In certain embodiments, the anti-PD-1 active agent is administered before the anti-LAG-3 active agent and after the anti-TIM-3 active agent.

In certain embodiments, the anti-TIM-3 active agent is administered concurrently with the anti-PD-1 active agent. In certain embodiments, the anti-TIM-3 active agent is administered concurrently with the anti-LAG-3 active agent. In certain embodiments, the anti-TIM-3 active agent is administered concurrently with the anti-PD-1 active agent and the anti-LAG-3 active agent. In certain embodiments, the anti-TIM-3 active agent is administered before the anti-PD-1 active agent. In certain embodiments, the anti-TIM-3 active agent is administered before the anti-LAG-3 active agent. In certain embodiments, the anti-TIM-3 active agent is administered before the anti-PD-1 active agent and the anti-LAG-3 active agent. In certain embodiments, the anti-TIM-3 active agent is administered after the anti-PD-1 active agent. In certain embodiments, the anti-TIM-3 active agent is administered after the anti-LAG-3 active agent. In certain embodiments, the anti-TIM-3 active agent is administered after the anti-PD-1 active agent and the anti-LAG-3 active agent. In certain embodiments, the anti-TIM-3 active agent is administered before the anti-PD-1 active agent and after the anti-LAG-3 active agent. In certain embodiments, the anti-TIM-3 active agent is administered before the anti-LAG-3 active agent and after the anti-PD-1 active agent.

In certain embodiments, the anti-LAG-3 active agent is administered concurrently with the anti-TIM-3 active agent. In certain embodiments, the anti-LAG-3 active agent is administered concurrently with the anti-PD-1 active agent. In certain embodiments, the anti-LAG-3 active agent is administered concurrently with the anti-TIM-3 active agent and the anti-PD-1 active agent. In certain embodiments, the anti-LAG-3 active agent is administered before the anti-TIM-3 active agent. In certain embodiments, the anti-LAG-3 active agent is administered before the anti-PD-1 active agent. In certain embodiments, the anti-LAG-3 active agent is administered before the anti-TIM-3 active agent and the anti-PD-1 active agent. In certain embodiments, the anti-LAG-3 active agent is administered after the anti-TIM-3 active agent. In certain embodiments, the anti-LAG-3 active agent is administered after the anti-PD-1 active agent. In certain embodiments, the anti-LAG-3 active agent is administered after the anti-TIM-3 active agent and the anti-PD-1 active agent. In certain embodiments, the anti-LAG-3 active agent is administered before the anti-TIM-3 active agent and after the anti-PD-1 active agent. In certain embodiments, the anti-LAG-3 active agent is administered before the anti-PD-1 active agent and after the anti-TIM-3 active agent.

### Anti-PD-1 Administration

In certain embodiments, the combination therapies, methods, and kits of the present disclosure comprise a therapeutically effective amount of an anti-PD-1 active agent.

In certain embodiments, the anti-PD-1 active agent (e.g., anti-PD-1 antibody or antibody fragment) is administered (or is formulated for administration) at a dose of at least about 1 mg, at least about 5 mg, at least about 10 mg, at least about 20 mg, at least about 50 mg, at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, or at least about 1000 mg of at least one anti-PD-1 active agent.

In certain embodiments, the anti-PD-1 active agent (e.g., anti-PD-1 antibody or antibody fragment) is administered (or is formulated for administration) at a dose from about 1 to about 5 mg, from about 5 to about 10 mg, from about 10 to about 20 mg, from about 20 to about 50 mg, from about 50 to about 100 mg, from about 100 to about 150 mg, from about 150 to about 200 mg, from about 175 to about 225 mg, from about 200 to about 250 mg, from about 225 to about 275 mg, from about 250 to about 300 mg, from about 275 to about 325 mg, from about 300 to about 350 mg, from about 325 to about 375 mg, from about 350 to about 400 mg, from about 375 to about 425 mg, from about 400 to about 450 mg, from about 425 to about 475 mg, from about 450 to about 500 mg, from about 475 to about 525 mg, from about 500 to about 550 mg, from about 525 to about 575 mg, from about 550 to about 600 mg, from about 575 to about 625 mg, from about 600 to about 650 mg, from about 625 to about 675 mg, from about 650 to about 700 mg, from about 675 to about 725 mg, from about 700 to about 750 mg, from about 725 to about 775 mg, from about 750 to about 800 mg, from about 775 to about 825 mg, from about 800 to about 850 mg, from about 825 to about 875 mg, from about 850 to about 900 mg, from about 875 to about 925 mg, from about 900 to about 950 mg, from about 925 to about 975 mg, from about 950 to about 1000 mg, from about 1000 to about 1050 mg, or from about 1000 to about 1100 mg of at least one anti-PD-1 active agent. In certain embodiments, the anti-PD-1 active agent (e.g., anti-PD-1 antibody or antibody fragment) is administered (or is formulated for administration) at a dose from about 300 to about 600 mg. In certain embodiments, the anti-PD-1 active agent (e.g., anti-PD-1 antibody or antibody fragment) is administered (or is formulated for administration) at a dose from about 375 to about 550 mg. In certain embodiments, the anti-PD-1 active agent (e.g., anti-PD-1 antibody or antibody fragment) is administered (or is formulated for administration) at a dose from about 450 to about 550 mg. In certain embodiments, the anti-PD-1 active agent (e.g., anti-PD-1 antibody or antibody fragment) is administered (or is formulated for administration) at a dose from about 375 to about 500 mg. In certain embodiments, the anti-PD-1 active agent (e.g., anti-PD-1 antibody or antibody fragment) is administered (or is formulated for administration) at a dose of about 375 mg. In certain embodiments, the anti-PD-1 active agent (e.g., anti-PD-1 antibody or antibody fragment) is administered (or is formulated for administration) at a dose of about 500 mg.

In certain embodiments, the anti-PD-1 active agent (e.g., anti-PD-1 antibody or antibody fragment) is administered (or is formulated for administration) at a dose of at least about 0.01 mg/kg, at least about 0.05 mg/kg, at least about 0.1 mg/kg, at least about 0.2 mg/kg, at least about 0.5 mg/kg, at least about 1 mg/kg, at least about 2 mg/kg, at least about 3 mg/kg, at least about 5 mg/kg, at least about 10 mg/kg, at least about 20 mg/kg, at least about 30 mg/kg, at least about 50 mg/kg, at least about 75 mg/kg, at least about 100 mg/kg, at least about 125 mg/kg, at least about 150 mg/kg, or more than 150 mg/kg, of the subject's body weight.

In certain embodiments, the anti-PD-1 active agent (e.g., anti-PD-1 antibody or antibody fragment) is administered (or is formulated for administration) in a single dose. In certain embodiments, the anti-PD-1 active agent (e.g., anti-PD-1 antibody or antibody fragment) is administered (or is formulated for administration) in more than one dose. In certain embodiments, the anti-PD-1 active agent (e.g., anti-PD-1 antibody or antibody fragment) is administered (or is formulated for administration) in one or more dose on a repeating basis. In certain embodiments, the anti-PD-1 active agent (e.g., anti-PD-1 antibody or antibody fragment) is administered (or is formulated for administration) 3 times per day, 2 times per day, 1 time per day, 1 time every two days, 1 time every three days, 1 time per week, 2 times per week, 3 times per week, 4 times per week, 1 time every two weeks, 1 time every three weeks, 1 time every four weeks, 1 time every five weeks, 1 time every six weeks, 1 time per month, 2 times per month, 3 times per month, 4 times per month, 1 time every two months, or 1 time every three months. In certain embodiments, the anti-PD-1 active agent (e.g., anti-PD-1 antibody or antibody fragment) is administered (or is formulated for administration) 1 time every two weeks, 1 time every three weeks, or 1 time every four weeks. In certain embodiments, the anti-PD-1 active agent (e.g., anti-PD-1 antibody or antibody fragment) is administered (or is formulated for administration) 1 time every three weeks or 1 time every four weeks. In certain embodiments, the anti-PD-1 active agent (e.g., anti-PD-1 antibody or antibody fragment) is administered (or is formulated for administration) 1 time every three weeks. In certain embodiments, the anti-PD-1 active agent (e.g., anti-PD-1 antibody or antibody fragment) is administered (or is formulated for administration) 1 time every four weeks.

In certain embodiments, the anti-PD-1 active agent (e.g., anti-PD-1 antibody or antibody fragment) is administered (or is formulated for administration) intravenously.

Suitable pharmaceutical compositions comprising an anti-PD-1 active agent are disclosed in U.S. Patent Application No. 63/220,006, the disclosure of which is incorporated herein by reference (as well as any subsequent patent application that claims priority to U.S. 63/220,006) as related to pharmaceutical compositions comprising an anti-PD-1 active agent In certain embodiments, the anti-PD-1 active agent (e.g., anti-PD-1 antibody or antibody fragment, for example retifanlimab) is administered (or is formulated for administration) in a pharmaceutical compositions comprising acetate, sucrose, polysorbate 80 (“PS80”), and water. In certain embodiments, the anti-PD-1 active agent (e.g., anti-PD-1 antibody or antibody fragment, for example retifanlimab) is administered (or is formulated for administration) in a pharmaceutical composition comprising acetate, sucrose, polysorbate 80 (“PS80”), and water, and has a pH of about 4.0 to about 6.5. In certain embodiments, the concentration of the anti-PD-1 agent in the pharmaceutical composition is about 10 mg/mL to about 100 mg/mL.

In certain embodiments, the acetate is present at a concentration of about 5 mM to about 30 mM. In certain embodiments, the acetate comprises sodium acetate, or glacial acetic acid and sodium acetate.

In certain embodiments of such pharmaceutical compositions, retifanlimab is present at a concentration of about 10 mg/mL to about 100 mg/mL, about 20 mg/mL to about 30 mg/mL, or about 25 mg/mL. In certain embodiments, retifanlimab is present at a concentration of about 10 mg/mL to about 100 mg/mL. In certain embodiments, retifanlimab is present at a concentration of about 20 mg/mL to about 30 mg/mL. In certain embodiments, retifanlimab is present at a concentration of about 25 mg/mL.

In certain embodiments of such pharmaceutical compositions, the acetate comprises glacial acetic acid at a concentration of about 0.05 mg/mL to about 0.35 mg/mL and sodium acetate trihydrate at a concentration of about 0.80 mg/mL to about 2.0 mg/mL. In certain embodiments, the acetate comprises glacial acetic acid at a concentration of about 0.18 mg/mL and sodium acetate trihydrate at a concentration of about 0.95 mg/mL.

In certain embodiments of such pharmaceutical compositions, the sucrose is present at a concentration of about 80 mg/mL to about 100 mg/mL, or about 90 mg/mL.

In certain embodiments of such pharmaceutical compositions, the PS80 is present at a concentration of about 0.08 mg/mL to about 0.15 mg/mL, or about 0.1 mg/mL.

In certain embodiments of such pharmaceutical compositions, the pharmaceutical composition has a pH of about 4.5 to about 5.7, or about 5.1.

In other embodiments, the anti-PD-1 active agent, for example retifanlimab, pharmaceutical composition comprises:
a) about 5 mM to about 30 mM acetate, about 50 mg/mL to about 130 mg/mL of sucrose, about 0.02 mg/mL to about 0.6 mg/mL of PS80, and water, wherein the composition has a pH of about 4.0 to about 6.5; or
b) about 7.5 mM to about 20 mM acetate, about 50 mg/mL to about 130 mg/mL of sucrose, about 0.05 mg/mL to about 0.6 mg/mL of PS80, and water, wherein the composition has a pH of about 4.0 to about 6.5; or
c) about 9 mM to about 11 mM acetate, about 76 mg/mL to about 104 mg/mL of sucrose, about 0.08 mg/mL to about 0.53 mg/mL of PS80, and water, wherein the composition has a pH of about 4.5 to about 5.7; or
d) about 9 mM to about 11 mM acetate, about 80 mg/mL to about 100 mg/mL of sucrose, about 0.08 mg/mL to about 0.15 mg/mL of PS80, and water, wherein the composition has a pH of about 4.5 to about 5.7.

In other embodiments, the anti-PD-1 active agent, for example retifanlimab, pharmaceutical composition comprises: about 25 mg/mL of retifanlimab, about 0.18 mg/mL of glacial acetic acid, about 0.95 mg/mL of sodium acetate trihydrate, about 90 mg/mL of sucrose, about 0.1 mg/mL of PS80, and water, wherein the composition has a pH of about 4.8 to about 5.4.

### Anti-TIM-3 Administration

In certain embodiments, the combination therapies, methods, and kits of the present disclosure comprise a therapeutically effective amount of an anti-TIM-3 active agent.

In certain embodiments, the anti-TIM-3 active agent (e.g., anti-TIM-3 antibody or antibody fragment) is administered (or is formulated for administration) at a dose of at least about 1 mg, at least about 5 mg, at least about 10 mg, at least about 20 mg, at least about 50 mg, at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, or at least about 1000 mg of at least one anti-TIM-3 active agent.

In certain embodiments, the anti-TIM-3 active agent (e.g., anti-TIM-3 antibody or antibody fragment) is administered (or is formulated for administration) at a dose from about 1 to about 5 mg, from about 5 to about 10 mg, from about 10 to about 20 mg, from about 20 to about 50 mg, from about 50 to about 100 mg, from about 100 to about 150 mg, from about 150 to about 200 mg, from about 175 to about 225 mg, from about 200 to about 250 mg, from about 225 to about 275 mg, from about 250 to about 300 mg, from about 275 to about 325 mg, from about 300 to about 350 mg, from about 325 to about 375 mg, from about 350 to about 400 mg, from about 375 to about 425 mg, from about 400 to about 450 mg, from about 425 to about 475 mg, from about 450 to about 500 mg, from about 475 to about 525 mg, from about 500 to about 550 mg, from about 525 to about 575 mg, from about 550 to about 600 mg, from about 575 to about 625 mg, from about 600 to about 650 mg, from about 625 to about 675 mg, from about 650 to about 700 mg, from about 675 to about 725 mg, from about 700 to about 750 mg, from about 725 to about 775 mg, from about 750 to about 800 mg, from about 775 to about 825 mg, from about 800 to about 850 mg, from about 825 to about 875 mg, from about 850 to about 900 mg, from about 875 to about 925 mg, from about 900 to about 950 mg, from about 925 to about 975 mg, from about 950 to about 1000 mg, from about 1000 to about 1050 mg, or from about 1000 to about 1100 mg of at least one anti-TIM-3 active agent. In certain embodiments, the anti-TIM-3 active agent (e.g., anti-TIM-3 antibody or antibody fragment) is administered (or is formulated for administration) at a dose from about 300 to about 1000 mg. In certain embodiments, the anti-TIM-3 active agent (e.g., anti-TIM-3 antibody or antibody fragment) is administered (or is formulated for administration) at a dose from about 350 to about 500 mg. In certain embodiments, the anti-TIM-3 active agent (e.g., anti-TIM-3 antibody or antibody fragment) is administered (or is formulated for administration) at a dose from about 350 to about 450 mg. In certain embodiments, the anti-TIM-3 active agent (e.g., anti-TIM-3 antibody or antibody fragment) is administered (or is formulated for administration) at a dose of about 400 mg, about 500 mg, or about 1000 mg. In certain embodiments, the anti-TIM-3 active agent (e.g., anti-TIM-3 antibody or antibody fragment) is administered (or is formulated for administration) at a dose of about 400 mg. In certain embodiments, the anti-TIM-3 active agent (e.g., anti-TIM-3 antibody or antibody fragment) is administered (or is formulated for administration) at a dose of about 500 mg. In certain embodiments, the anti-TIM-3 active agent (e.g., anti-TIM-3 antibody or antibody fragment) is administered (or is formulated for administration) at a dose of about 1000 mg.

In certain embodiments, the anti-TIM-3 active agent (e.g., anti-TIM-3 antibody or antibody fragment) is administered (or is formulated for administration) at a dose of at least about 0.01 mg/kg, at least about 0.05 mg/kg, at least about 0.1 mg/kg, at least about 0.2 mg/kg, at least about 0.5 mg/kg, at least about 1 mg/kg, at least about 2 mg/kg, at least about 3 mg/kg, at least about 5 mg/kg, at least about 10 mg/kg, at least about 20 mg/kg, at least about 30 mg/kg, at least about 50 mg/kg, at least about 75 mg/kg, at least about 100 mg/kg, at least about 125 mg/kg, at least about 150 mg/kg, or more than 150 mg/kg, of the subject's body weight.

In certain embodiments, the anti-TIM-3 active agent (e.g., anti-TIM-3 antibody or antibody fragment) is administered (or is formulated for administration) in a single dose. In certain embodiments, the anti-TIM-3 active agent (e.g., anti-TIM-3 antibody or antibody fragment) is administered (or is formulated for administration) in more than one dose. In certain embodiments, the anti-TIM-3 active agent (e.g., anti-TIM-3 antibody or antibody fragment) is administered (or is formulated for administration) in one or more dose on a repeating basis. In certain embodiments, the anti-TIM-3 active agent (e.g., anti-TIM-3 antibody or antibody fragment) is administered (or is formulated for administration) 3 times per day, 2 times per day, 1 time per day, 1 time every two days, 1 time every three days, 1 time per week, 2 times per week, 3 times per week, 4 times per week, 1 time every two weeks, 1 time every three weeks, 1 time every four weeks, 1 time every five weeks, 1 time every six weeks, 1 time per month, 2 times per month, 3 times per month, 4 times per month, 1 time every two months, or 1 time every three months. In certain embodiments, the anti-TIM-3 active agent (e.g., anti-TIM-3 antibody or antibody fragment) is administered (or is formulated for administration) 1 time every week, 1 time every two weeks, or 1 time every three weeks. In certain embodiments, the anti-TIM-3 active agent (e.g., anti-TIM-3 antibody or antibody fragment) is administered (or is formulated for administration) 1 time every two weeks or 1 time every three weeks. In certain embodiments, the anti-TIM-3 active agent (e.g., anti-TIM-3 antibody or antibody fragment) is administered (or is formulated for administration) 1 time every two weeks. In certain embodiments, the anti-TIM-3 active agent (e.g., anti-TIM-3 antibody or antibody fragment) is administered (or is formulated for administration) 1 time every three weeks.

In certain embodiments, the anti-TIM-3 active agent (e.g., anti-TIM-3 antibody or antibody fragment) is administered (or is formulated for administration) intravenously.

In certain embodiments, the anti-TIM-3 active agent (e.g., anti-TIM-3 antibody or antibody fragment) is administered (or is formulated for administration) in a pharmaceutical composition comprising: sodium citrate, sucrose, arginine, polysorbate 80, and has a pH 6.0. In certain embodiments, the concentration of the anti-TIM-3 agent in the pharmaceutical composition is from about 45 to about 55 mg/mL. In certain embodiments, the concentration of the anti-TIM-3 agent in the pharmaceutical composition is about 50 mg/mL.

### Anti-LAG-3 Administration

In certain embodiments, the combination therapies, methods, and kits of the present disclosure comprise a therapeutically effective amount of an anti-LAG-3 active agent.

In certain embodiments, the anti-LAG-3 active agent (e.g., anti-LAG-3 antibody or antibody fragment) is administered (or is formulated for administration) at a dose of at least about 1 mg, at least about 5 mg, at least about 10 mg, at least about 20 mg, at least about 50 mg, at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, or at least about 1000 mg of at least one anti-LAG-3 active agent.

In certain embodiments, the anti-LAG-3 active agent (e.g., anti-LAG-3 antibody or antibody fragment) is administered (or is formulated for administration) at a dose from about 1 to about 5 mg, from about 5 to about 10 mg, from about 10 to about 20 mg, from about 20 to about 50 mg, from about 50 to about 100 mg, from about 100 to about 150 mg, from about 150 to about 200 mg, from about 175 to about 225 mg, from about 200 to about 250 mg, from about 225 to about 275 mg, from about 250 to about 300 mg, from about 275 to about 325 mg, from about 300 to about 350 mg, from about 325 to about 375 mg, from about 350 to about 400 mg, from about 375 to about 425 mg, from about 400 to about 450 mg, from about 425 to about 475 mg, from about 450 to about 500 mg, from about 475 to about 525 mg, from about 500 to about 550 mg, from about 525 to about 575 mg, from about 550 to about 600 mg, from about 575 to about 625 mg, from about 600 to about 650 mg, from about 625 to about 675 mg, from about 650 to about 700 mg, from about 675 to about 725 mg, from about 700 to about 750 mg, from about 725 to about 775 mg, from about 750 to about 800 mg, from about 775 to about 825 mg, from about 800 to about 850 mg, from about 825 to about 875 mg, from about 850 to about 900 mg, from about 875 to about 925 mg, from about 900 to about 950 mg, from about 925 to about 975 mg, from about 950 to about 1000 mg, from about 1000 to about 1050 mg, or from about 1000 to about 1100 mg of at least one anti-LAG-3 active agent. In certain embodiments, the anti-LAG-3 active agent (e.g., anti-LAG-3 antibody or antibody fragment) is administered (or is formulated for administration) at a dose from about 300 to about 800 mg. In certain embodiments, the anti-LAG-3 active agent (e.g., anti-LAG-3 antibody or antibody fragment) is administered (or is formulated for administration) at a dose from about 350 to about 750 mg. In certain embodiments, the anti-LAG-3 active agent (e.g., anti-LAG-3 antibody or antibody fragment) is administered (or is formulated for administration) at a dose from about 300 to about 400 mg. In certain embodiments, the anti-LAG-3 active agent (e.g., anti-LAG-3 antibody or antibody fragment) is administered (or is formulated for administration) at a dose of about 350 mg, about 450 mg, or about 750 mg. In certain embodiments, the anti-LAG-3 active agent (e.g., anti-LAG-3 antibody or antibody fragment) is administered (or is formulated for administration) at a dose of about 350 mg. In certain embodiments, the anti-LAG-3 active agent (e.g., anti-LAG-3 antibody or antibody fragment) is administered (or is formulated for administration) at a dose of about 450 mg. In certain embodiments, the anti-LAG-3 active agent (e.g., anti-LAG-3 antibody or antibody fragment) is administered (or is formulated for administration) at a dose of about 750 mg.

In certain embodiments, the anti-LAG-3 active agent (e.g., anti-LAG-3 antibody or antibody fragment) is administered (or is formulated for administration) at a dose of at least about 0.01 mg/kg, at least about 0.05 mg/kg, at least about 0.1 mg/kg, at least about 0.2 mg/kg, at least about 0.5 mg/kg, at least about 1 mg/kg, at least about 2 mg/kg, at least about 3 mg/kg, at least about 5 mg/kg, at least about 10 mg/kg, at least about 20 mg/kg, at least about 30 mg/kg, at least about 50 mg/kg, at least about 75 mg/kg, at least about 100 mg/kg, at least about 125 mg/kg, at least about 150 mg/kg, or more than 150 mg/kg, of the subject's body weight.

In certain embodiments, the anti-LAG-3 active agent (e.g., anti-LAG-3 antibody or antibody fragment) is administered (or is formulated for administration) in a single dose. In certain embodiments, the anti-LAG-3 active agent (e.g., anti-LAG-3 antibody or antibody fragment) is administered (or is formulated for administration) in more than one dose. In certain embodiments, the anti-LAG-3 active agent (e.g., anti-LAG-3 antibody or antibody fragment) is administered (or is formulated for administration) in one or more dose on a repeating basis. In certain embodiments, the anti-LAG-3 active agent (e.g., anti-LAG-3 antibody or antibody fragment) is administered (or is formulated for administration) 3 times per day, 2 times per day, 1 time per day, 1 time every two days, 1 time every three days, 1 time per week, 2 times per week, 3 times per week, 4 times per week, 1 time every two weeks, 1 time every three weeks, 1 time every four weeks, 1 time every five weeks, 1 time every six weeks, 1 time per month, 2 times per month, 3 times per month, 4 times per month, 1 time every two months, or 1 time every three months. In certain embodiments, the anti-LAG-3 active agent (e.g., anti-LAG-3 antibody or antibody fragment) is administered (or is formulated for administration) 1 time every week, 1 time every two weeks, or 1 time every three weeks. In certain embodiments, the anti-LAG-3 active agent (e.g., anti-LAG-3 antibody or antibody fragment) is administered (or is formulated for administration) 1 time every two weeks or 1 time every three weeks. In certain embodiments, the anti-LAG-3 active agent (e.g., anti-LAG-3 antibody or antibody fragment) is administered (or is formulated for administration) 1 time every two weeks. In certain embodiments, the anti-LAG-3 active agent (e.g., anti-LAG-3 antibody or antibody fragment) is administered (or is formulated for administration) 1 time every three weeks.

In certain embodiments, the anti-LAG-3 active agent (e.g., anti-LAG-3 antibody or antibody fragment) is administered (or is formulated for administration) intravenously.

In certain embodiments, the anti-LAG-3 active agent (e.g., anti-LAG-3 antibody or antibody fragment) is administered (or is formulated for administration) in a pharmaceutical composition comprising: sodium acetate, trehalose, polysorbate 80, and has a pH 5.5. In certain embodiments, the concentration of the anti-LAG-3 agent in the pharmaceutical composition is from about 45 to about 55 mg/mL. In certain embodiments, the concentration of the anti-LAG-3 agent in the pharmaceutical composition is about 50 mg/mL.

### III. Definitions

At various places in the present disclosure, substituents, or properties of compounds of the present disclosure are disclosed in groups or in ranges. It is intended that the present disclosure comprise each and every individual or sub-combination of the members of such groups and ranges, and that such groups or ranges include the endpoints. By way of nonlimiting example, if a group or range is from about 1 to about 10, then the group or range includes both the value of about 1 and the value of about 10.

Unless stated otherwise, the following terms and phrases have the meanings described below. The definitions are not meant to be limiting in nature and serve to provide a clearer understanding of certain aspects of the present disclosure.

Administering: As used herein, the term “administering” refers to providing a composition to a subject.

Agonist: As used herein, the term “agonist” refers to a ligand, including but not limited to an antigen binding protein such as an antibody or antibody fragment, which upon contact with a receptor causes one or more of the following (1) stimulates or activates the receptor, (2) enhances, increases or promotes, induces or prolongs an activity, function or presence of the receptor, (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor, and/or (4) enhances, increases, promotes, or induces the expression of the receptor. Agonist activity can be measured in vitro by various assays known in the art such as, but not limited to, measurement of cell signaling, cell proliferation, immune cell activation markers, or cytokine production. Agonist activity can also be measured in vivo by various assays that measure surrogate end points such as, but not limited to the measurement of T-cell proliferation or cytokine production.

Antagonist: As used herein, the term “antagonist” refers to a ligand, including but not limited to an antigen binding protein such as an antibody or antibody fragment, which upon contact with a receptor causes one or more of the following (1) attenuates, blocks, or inactivates the receptor and/or blocks activation of a receptor by its natural ligand, (2) reduces, decreases, or shortens the activity, function or presence of the receptor, and/or (3) reduces, decrease, abrogates the expression of the receptor. Antagonist activity can be measured in vitro by various assays known in the art such as, but not limited to, measurement of an increase or decrease in cell signaling, cell proliferation, immune cell activation markers, or cytokine production. Antagonist activity can also be measured in vivo by various assays that measure surrogate end points such as, but not limited to the measurement of T-cell proliferation or cytokine production.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a non-human primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Approximately/About: As used herein, the terms “approximately” and “about” are used interchangeably herein and refer to a value that is within +/−10% of the recited value as applied to one or more values of interest. In certain embodiments, the term refers to a range of values that fall within +/−10%, +/−9%, +/−8%, +/−7%, +/−6%, +/−5%, +/−4%, +/−3%, +/−2%, +/−1%, or less of the stated reference value, unless otherwise expressly stated or otherwise clearly evident from the context.

Binding affinity: As used herein, the term “binding affinity” describes the strength of the sum total of non-covalent interactions between a binding site of a molecule (e.g., an antibody) and a binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}) (e.g., a “lower affinity” referring to a larger K_{D}). Affinity can be measured and/or expressed in a number of ways known in the art, including, but not limited to: (i) equilibrium dissociation constant (K_{D}), which is calculated from the quotient of k-off/k-on; and (ii) equilibrium association constant (K_{A}), which is calculated from the quotient of k-on/k-off. “k-on” refers to the association rate constant of, e.g., an antibody to an antigen, and “k-off” refers to the dissociation rate constant of, e.g., an antibody to an antigen. The k-on and k-off can be determined by techniques known to those with skill in the art, such as Biacore Assay or KinExA.

Complete Response: As used herein, the term “complete response” or “CR” refers to the removal of all or substantially all target lesions in a target. In certain embodiments, CR refers to an about 80%, 85%, 90%, 91%, 92%, 93%, 94%, 95%, 96%, 97%, 98%, 99% or 100% decrease in the sum of the diameters of the target lesions (i.e., loss of lesions), relative to a baseline sum of diameters. In certain embodiments, CR refers to less than about 10%, 9%, 8%, 7%, 6%, 5%, 4%, 3%, 2%, 1% or less of the total lesion diameter remaining after treatment, relative to a baseline total lesion diameter. In certain embodiments, a complete response is evaluated according to Response Evaluation Criteria in Solid Tumors (RECIST) guidelines.

Epitope: As used herein, the term “epitope” refers to a localized region of an antigen to which an antibody (or fragment thereof) can bind. An epitope can be, for example, contiguous amino acids of a polypeptide (i.e., linear or contiguous epitope), or can also comprise multiple non-contiguous amino acids of a polypeptide chain that come together from at least two non-contiguous regions of a polypeptide to form an epitope region.

Epitope-binding fragment: As used herein, the term “epitope-binding fragment” describes a portion or fragment of an antibody capable of binding to an epitope.

Epitope-binding site: As used herein, the term “epitope-binding site” refers to a portion of a molecule comprising an epitope-binding fragment that is responsible for epitope binding of a target.

Feature: As used herein, a “feature” refers to a characteristic, a property, or a distinctive element of an identified subject.

Identity: As used herein, the terms “identity” and “percent identity” refer to the overall relatedness between the sequences of polymeric molecules, e.g., polynucleotide molecules or polypeptide molecules. Calculation of the percent identity of two sequences can be performed by aligning the two sequences for optimal comparison purposes (e.g., gaps can be introduced in one or both sequences for optimal alignment), and then comparing the aligned sequences. The percent identity between the two sequences is a function of the number of identical positions shared by the sequences relative to the total length of the sequences, taking into account the number and length of any gaps introduced for optimal alignment of the sequences. In certain embodiments, the comparison of sequences and determination of percent identity between two sequences can be accomplished using a mathematical algorithm. For example, the percent identity between two sequences can be determined using methods such as those described in Karlin et al., PNAS (1990) 87: 2264-2268; Karlin et al., PNAS (1993) 90: 5873-5877; Carillo et al., Applied Math. (1988) 48:1073; Meyers et al., CABIOS (1989) 4:11-17 (as incorporated into the ALIGN program); Lesk et al., Computational Molecular Biology, Oxford University Press, New York, 1988; Smith et al., Biocomputing: Informatics and Genome Projects, Academic Press, New York, 1993; Von Heinje et al., Sequence Analysis in Molecular Biology, Academic Press, 1987; Griffin et al., Computer Analysis of Sequence Data, Part I, Humana Press, New Jersey, 1994; and Gribskov et al., Sequence Analysis Primer, Stockton Press, New York, 1991. As one example, the percent identity between two sequences can be determined using the GAP program in the GCG software package using an NWSgapdna.CMP matrix. Techniques and tools for determining percent identity are also codified in publicly available computer programs, including but not limited to, GCG program package, FASTA, P-BLAST, N-BLAST, and X-BLAST (see, e.g., National Center for Biotechnology Information (NCBI) on the worldwide web, ncbi.nlm.nih.gov).

Partial Response: As used herein, the terms “partial response” or “PR” refer to a decrease in tumor progression in a subject as indicated by a decrease in the sum of the diameters of the target lesions, relative to a baseline sum diameters. In certain embodiments, PR refers to at least a 30% decrease in the sum of diameters of target lesions, relative to a baseline sum diameters. In certain embodiments, PR refers to a decrease of at least about 30%, 35%, 40%, 45%, 50%, 55%, 60%, 65%, 70%, 75%, 80%, 85%, or at least about 90% decrease in the sum of the diameters of the target lesions relative to a baseline sum of diameters. In certain embodiments, a partial response is evaluated according to Response Evaluation Criteria in Solid Tumors (RECIST) guidelines.

Pharmaceutically acceptable: As used herein, the terms “pharmaceutically acceptable” or “therapeutically acceptable” are used to describe compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the terms “pharmaceutically acceptable excipient” or “therapeutically acceptable excipient,” refer to an ingredient in a composition other than an active agent described herein (e.g., a vehicle capable of suspending, carrying, or encapsulating the active agent) and having the properties of being substantially nontoxic and non-inflammatory in a subject.

Progressive Disease: As used herein, the terms “progressive disease” or “PD” refer to an increase in tumor progression in a subject as indicated by an increase in the sum of the diameters of the target lesions, relative to a baseline sum diameters. In certain embodiments, PD refers to at least a 20% increase in the sum of diameters of target lesions, relative to a baseline sum diameters. In certain embodiments, PR refers to an increase of at least about 20%, 25%, 30%, 35%, 40%, 45%, 50%, 55%, 60%, 65%, 70%, 75%, 80%, 85%, or at least about 90% increase in the sum of the diameters of the target lesions relative to a baseline sum of diameters. In certain embodiments, PD refers to an appearance of one or more new lesions. In certain embodiments, a progressive disease is evaluated according to Response Evaluation Criteria in Solid Tumors (RECIST) guidelines.

Stable Disease: As used herein, the terms “stable disease” or “SD” refer to a tumor progression that has neither sufficient shrinkage to be considered a partial response (PR) nor sufficient increase to be considered a progressive disease (PD). In certain embodiments, SD refers to less than a 30% decrease in the sum of diameters of target lesions, relative to a baseline sum diameters. In certain embodiments, SD refers to a decrease of less than about 30%, 25%, 20%, 15%, 10%, or 5%, or a 0% decrease in the sum of the diameters of the target lesions relative to a baseline sum of diameters. In certain embodiments, SD refers to less than a 20% increase in the sum of diameters of target lesions, relative to a baseline sum diameters. In certain embodiments, SD refers to an increase of less than about 20%, 15%, 10%, or 5%, or a 0% increase in the sum of the diameters of the target lesions relative to a baseline sum of diameters. In certain embodiments, a stable disease is evaluated according to Response Evaluation Criteria in Solid Tumors (RECIST) guidelines.

Subject: As used herein, the term “subject” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Therapeutically effective amount: As used herein, the term “therapeutically effective amount” of an agent is an amount sufficient to effect beneficial or desired results (e.g., biological, medical, or clinical results). As such, an effective amount depends upon the context in which it is being applied (e.g., route of administration, seriousness of the condition, biochemistry, and medical history of subject, etc.), and can be determined by standard clinical techniques by those with skill in the art (e.g., extrapolated from dose-response curves derived from testing). For example, in the context of administering an agent that treats cancer, an effective amount of an agent can be an amount sufficient to achieve treatment of the cancer, as compared to the response obtained without administration of the agent.

Treating: As used herein, the term “treating” refers to partially or completely alleviating, ameliorating, improving, relieving, preventing, delaying onset of, inhibiting progression of, reducing severity of, and/or reducing incidence of one or more symptoms or features of a particular disease, disorder, and/or condition, for example cancer. Treatment may be administered to a subject who does not exhibit signs of a disease, disorder, and/or condition and/or to a subject who exhibits only early signs of a disease, disorder, and/or condition for the purpose of decreasing the risk of developing symptoms or pathology associated with the disease, disorder, and/or condition, for example, decrease in tumor size, decrease in the presence of circulating tumor cells, reduction or prevention of metastases, slowing or arresting tumor growth and/or preventing or delaying tumor recurrence or relapse. Treatment can also include an improvement of one or more characteristics associated with a disease, disorder and/or condition over baseline, including an improvement over a measurement or observation made prior to initiation of treatment according to the methods of the present disclosure.

### General Considerations

Those skilled in the art will recognize or be able to ascertain using no more than routine experimentation, one or more equivalents to the specific embodiments in accordance with the present disclosure described herein. The scope of the present disclosure is not intended to be limited to the above Description.

Articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure can include embodiments in which exactly one member of the group is present in, employed in, or otherwise relevant to a given product or process. The present disclosure can include embodiments in which more than one, or the entire group members are present in, employed in, or otherwise relevant to a given product or process.

The term “comprising” is intended to be open and permits but does not require the inclusion of additional elements or steps. When the term “comprising” is used herein, the term “consisting of” and “consisting essentially of” is also encompassed and disclosed.

The abbreviation, “e.g.,” is derived from the Latin exempli gratia, and is used herein to indicate a non-limiting example. Thus, the abbreviation “e.g.,” is synonymous with the term “for example.” The abbreviation, “i.e.,” is derived from the Latin id est, and is used herein to indicate a non-limiting rewording or clarification. Thus, the abbreviation “i.e.,” is synonymous with the term “that is.”

Any particular embodiment of the present disclosure that falls within the prior art may be explicitly excluded from any one or more of the claims. Any particular embodiment of the agents, combination therapies, methods, and/or compositions of the present disclosure can be excluded from any one or more claims, for any reason, whether or not related to the existence of prior art.

The present specification will control in instances where publications, patent applications, patents, and other references mentioned herein are incorporated by reference and are in conflict with the present specification.

Section headings, materials, methods, and examples are illustrative only and not intended to be limiting.

### EXAMPLES

### Example 1. Combination Therapy Study of Anti-PD-1 Active Agent, Anti-TIM-3 Active Agent, and Anti-LAG-3 Active Agent in Urothelial Carcinoma

A study is being conducted to investigate the biological and therapeutic outcomes for selected combination therapies, including the combination of an anti-PD-1 active agent, an anti-TIM-3 active agent, and an anti-LAG-3 active agent. The study is also being conducted to evaluate the clinical and biological effects of blocking additional key checkpoint pathways, LAG-3 and TIM-3, which have been implicated in the lack of/loss of response to PD-1 inhibitors.

A cohort of 18 patients was selected with muscle-invasive urothelial carcinoma of the bladder who are undergoing (or have previously undergone) radical cystectomy and are either cisplatin-ineligible or refuse cisplatin therapy.

The anti-PD-1 antibody for the study is INCMGA00012 (i.e., retifanlimab). The anti-TIM-3 antibody for the study is Antibody A. The anti-LAG-3 antibody for the study is Antibody B. The order of administration of study drugs is: (1) INCMGA00012, followed by (2) Antibody B, followed by (3) Antibody A.

a. Dose Selection for INCMGA00012

INCMGA00012 (i.e., retifanlimab) is being administered at 500 mg, once every four weeks (i.e., Q4W), by IV infusion over about 30 minutes on Day 1 of each 28-day cycle. The dosage formulation includes 25 mg/mL in liquid formulation for a 500 mg dose.

The selection of this dose is based on modeling of clinical PK data from a first-in-human monotherapy study, in which 37 participants were treated with doses of 1 mg/kg Q2W, 3 mg/kg Q2W, 3 mg/kg Q4W, 10 mg/kg Q2W, and 10 mg/kg Q4W. Pharmacokinetic data was obtained from 15 participants who received INCMGA00012 500 mg Q4W. The observed AUC_{0-∞} for 500 mg Q4W was close to the steady-state AUC₀₋ₜ based on the population PK analysis of weight-based doses, as is the estimated clearance. The 500 mg Q4W dose had approximately 58% probability to obtain a steady-state trough plasma concentration≥21 μg/mL, which is associated with maximum target engagement and greatest probability of efficacy. Based on these observations, 500 mg Q4W was chosen as the dose regimen.

b. Dose Selection for Antibody B

Antibody B is being administered at 350 mg, once every two weeks (i.e., Q2W), by IV infusion over about 30 minutes on Day 1 of each 14-day cycle. The dosage formulation includes 50 mg/mL in liquid formulation for a 350 mg dose.

The selection of this dose is based on modeling of clinical PK data from a human monotherapy study, in which 22 participants were treated with IV doses of 25, 75, 250, 350, and 750 mg Q2W in an open-label, nonrandomized, dose escalation, and cohort-expansion study. All doses were well tolerated. Antibody B PK was linear after the first dose and showed moderate to high inter-individual variability. At a dose of 350 mg Q2W (N=3), the steady-state AUC was 30,200 μg/mL·h. The dose of 350 mg Q2W was chosen based on data showing that: (i) receptors on the surface of the cells were fully occupied at trough concentration of post 350 mg Q2W dosing, and (ii) the dose is pharmacologically active, as measured by an increase in peripheral T-cell activation observed in doses greater than 250 mg.

c. Dose Selection for Antibody A

Antibody A is being administered at 400 mg, once every two weeks (i.e., Q2W), by IV infusion over about 30 minutes on Day 1 of each 14-day cycle. The dosage formulation includes 50 mg/mL in liquid formulation for a 400 mg dose.

The selection of this dose is based on modeling of clinical PK data from a human monotherapy study, in which 38 participants were treated with IV doses of 10, 30, 100, 200, 400, 800, and 1600 mg Q2W in the open-label, nonrandomized, dose-escalation, and cohort expansion study. All doses were well tolerated. Antibody A showed supra-proportional PK after first dose from 30 mg to 800 mg Q2W and low inter-individual variability on PK exposures. At a dose of 400 mg Q2W, the mean steady-state AUC determined in 4 participants was 43,400 μg/mL·h. The dose of 400 mg was chosen based on data showing that TIM-3 receptor was fully occupied on the surface of circulating monocytes at trough concentrations post at 400 mg Q2W dosing. In addition, changes in pharmacodynamic markers suggest this dose to be pharmacologically active.

d. Patient Selection

Tumor tissue from each patient was tested to determine PD-L1 CPS score. Participants were randomized into treatment groups, with PD-L1 CPS<10 or PD-L1 CPS≥10 being evenly randomized into treatment groups.

Patient inclusion criteria included: (i) Men or women aged 18 years or older; (ii) Histologically confirmed transitional cell urothelial carcinoma-Participants with mixed histologies were required to have a dominant (i.e., 50% at least) transitional cell pattern; (iii) Clinical stage T2-T3b, N0, M0 muscle invasive urothelial carcinoma by CT (or MRI) (Stage II-IIIA per AJCC 2018)—Participants with concomitant upper tract tumors were excluded, though previous history of surgery for upper tract tumors was allowed, provided that it was a noninvasive pT (i.e., pT<2N0M0 stage); (iv) Ineligibility for cisplatin therapy, per modified Galsky criteria with exclusion of ECOG PS 2 participants (see Appendix D); (v) Refusal of cisplatin-based therapy; (vi) Eligible for radical cystectomy; (vii) ECOG PS of 0 or 1; and (viii) Pretreatment tumor biopsy must have been a tumor block or 20 unstained slides from biopsy of primary tumor containing at least 20% tumor.

Patient exclusion criteria included: (i) Participation in any other study in which receipt of an investigational study drug or device occurred within 28 days or 5 half-lives (whichever is longer) before first dose in this study; (ii) Previously received systemic therapy for bladder cancer or received prior treatment with checkpoint inhibitor agents (such as anti-PD-1, anti-PDL-1, anti-PDL-2, or anti-CTLA 4); (iii) Evidence of measurable nodal or metastatic disease; (iv) Concurrent anticancer therapy (e.g., chemotherapy, radiation therapy, surgery, immunotherapy, biologic therapy, hormonal therapy, investigational therapy, intravesical therapy or tumor embolization); (v) Had major surgery within 4 weeks before enrollment (CIDI); (vi) Had known additional malignancy other than miUBC that was progressing or required active treatment, or a history of other malignancies within 2 years of study entry with the exception of cured basal cell or squamous cell carcinoma of the skin, superficial bladder cancer, prostate intraepithelial neoplasm, carcinoma in situ of the cervix, or other noninvasive or indolent malignancy, or cancers from which the participant had been disease-free for >1 year, after treatment with curative intent; (vii) Had active autoimmune disease requiring systemic immunosuppression with corticosteroids (>10 mg daily doses of prednisone or equivalent) or immunosuppressive drugs within 2 years of Day 1 of study treatment; (viii) Patients with active autoimmune disease requiring systemic immunosuppression in excess of physiologic maintenance doses of corticosteroids (>10 mg/day of prednisone or equivalent); (ix) Had known active hepatitis B or C or HIV, HBV, HCV, or hepatitis D virus coinfection; Has known carcinomatous meningitis; (x) Active infection requiring systemic antibiotics≤14 days from first dose of study drug; (xi) A known or suspected COVID-19 infection; (xii) Use of probiotics within 28 days from first dose of study drug; (xiii) Current use of prohibited medication (e.g., other anticancer therapies, immunosuppressive drugs or corticosteroids, systemic steroids, live vaccines, UGT1A9 inhibitors, warfarin, and systemic antibiotics); (xiv) Had not recovered to ≤Grade 1 from toxic effects of previous therapy and/or complications from previous surgical intervention before starting study therapy; (xv) History or presence of an abnormal, clinically meaningful ECG; (xvi) History of a gastrointestinal condition (e.g., inflammatory bowel disease, Crohn disease, ulcerative colitis) that may affect oral drug absorption; (xvii) Had received a live vaccine within 30 days of planned start of study therapy; (xvii) Impaired cardiac function or clinically significant cardiac disease; (xviii) Prior allogenic tissue/solid organ transplant; (xix) Participants with failed laboratory screenings, including (a) platelets<100×10⁹/L, (b) Hemoglobin<9 g/dL, (c) ANC<1.5×109/L, (d) ALT>2×ULN, (e) AST>2×ULN, (f) CrCl<30 mL/min, calculated by Cockcroft-Gault equation, (g) INR or PT>1.5×ULN (unless on therapeutic anticoagulants), and (h) aPTT>1.5×ULN; (xx) Evidence of interstitial lung disease or active, noninfectious pneumonitis; (xxi) Had known hypersensitivity to any of the study drugs, excipients, including mannitol or another monoclonal antibody which could not be controlled with standard measures (e.g., antihistamines and corticosteroids); and (xxii) Any ≥Grade 2 immune-related toxicity while receiving prior immunotherapy.

e. Study Objectives and Endpoints

A primary study endpoint for each treatment group is the change from baseline in CD8+ lymphocytes within resected tumor.

Secondary endpoints include: (i) Safety and tolerability, assessed by monitoring the frequency and severity of adverse events (AEs), including delay in cystectomy due to AEs; (ii) patient pCR rate, defined as percentage of participants with ypT0N0 in each treatment group; and (iii) Major pathological response, defined as residual ypT0/1/a/isN0M0.

Exploratory endpoints include: (i) Changes in tumor, tumor stroma, and tumor associated immune cell gene expression profile from baseline to each visit where the variable is measured and correlation with treatment outcomes; (ii) Changes in tumor, tumor stroma, and tumor associated immune cell protein and metabolic marker levels and spatial distribution from baseline to each visit where the variable is measured and correlation with treatment outcomes; (iii) Changes in plasma analytes, including cytokines and other known markers of inflammation and immune status, tumor markers, and markers associated with metabolism and nutritional status from baseline to each visit where the variable is measured and correlation with treatment outcomes; (iv) Biologic response rate for those with CPS<10 versus ≥10 in each treatment group; (v) Descriptive analyses between the combination/sequential treatment groups and monotherapy treatment groups; (vi) Pathological downstaging (i.e., ypT<2N0M0) in each treatment group; and (vii) The percentage of pathological downstaging in each treatment group will be calculated and its 80% CI will be estimated using the Clopper Pearson method (Voskuilen et al., 2019).

Efficacy analysis for the pCR rate and pathological downstage evaluations includes all participants who met the eligibility criteria, had at least 1 cycle of study treatment, and underwent cystectomy or withdrew for progression of disease. pCR is defined as pT0 and in situ cancer on the basis of histological evaluation of the TURBT and cystectomy samples by local institutional analysis. Major pathological response is defined as residual ypT0/1/a/isN0M0 on the basis of histological evaluation of the TURBT and cystectomy samples. Pathological downstaging is defined as ypT<2N0M0, and is conducted on the basis of evaluations of the TURBT and cystectomy samples by local institution analysis using the AJCC staging system 8th edition, 2017.

### Example 2. Combination Therapy Study of Anti-PD-1 Active Agent, Anti-TIM-3 Active Agent, and Anti-LAG-3 Active Agent in Melanoma

A study is being conducted to investigate the biological and therapeutic outcomes for selected combination therapies, including the combination of an anti-PD-1 active agent, an anti-TIM-3 active agent, and an anti-LAG-3 active agent. The study is also being conducted to evaluate the efficacy of the combination of an anti-PD-1 active agent, an anti-TIM-3 active agent, and an anti-LAG-3 active agent in participants with melanoma who have progressed or relapsed on or after anti-PDL-1 therapy as their most recent line of therapy.

A cohort is being selected of 15-30 patients with advanced malignancies (e.g., unresectable, metastatic, or adjuvant-treated melanoma) who have received prior treatment with therapy directed toward PD-1 and/or PDL-1, and have progressed or relapsed on or after the prior anti-PDL-1 therapy.

The study includes two phases, with Phase 1 having four parts:

(1a) Phase 1, Part 1—Uses a BOIN design to determine whether the doses selected for study are safe when used in the combination of Antibody B (350 mg Q2W) and Antibody A (400 mg Q2W) (n≤9) in patients with tumors for which PD-1 inhibitors are indicated, and who have failed PD-1-directed therapy. If the safety of doses tested cannot be established, then the dose level will be reduced (i.e., 250 mg Q2W for Antibody B and 200 mg Q2W for Antibody A);

(1b) Phase 1, Part 2—Following confirmation of the safety of the combination of Antibody B and Antibody A, the safety of the combination of Antibody B (350 mg Q2W)+Antibody A (400 mg Q2W)+INCMGA00012 (500 mg Q4W) will be tested (n≤9) in patients with tumors for which PD-1 inhibitors are indicated, and who have failed PD-1-directed therapy. If safety of doses tested cannot be established, the dose levels for Antibody B and Antibody A will be reduced (i.e., 250 mg Q2W for Antibody B and 200 mg Q2W for Antibody A); and

(1c) Phase 1, Part 3—Safety study on the following combination therapies in patients with tumors for which PD-1 inhibitors are indicated, and who have failed PD-1-directed therapy: (1c.i) Antibody B (450 mg Q3W)+INCMGA00012 (375 mg Q3W) (n˜6), with patients first receiving two 28-day cycles of 450 mg Antibody B, followed by the doublet combination for subsequent cycles; (1c.ii) Antibody B (750 mg Q3W)+INCMGA00012 (375 mg Q3W) (n˜6), with patients first receiving two 28-day cycles of 750 mg Antibody B, followed by the doublet combination for subsequent cycles; (1c.iii) Antibody A (500 mg Q3W)+INCMGA00012 (375 mg Q3W) (n˜6), with patients first receiving two 28-day cycles of 500 mg Antibody A, followed by the doublet combination for subsequent cycles; (1c.iv) Antibody A (1000 mg Q3W)+INCMGA00012 (375 mg Q3W) (n˜6), with patients first receiving two 28-day cycles of 1000 mg Antibody A, followed by the doublet combination for subsequent cycles.

(1d) Phase 1, Part 4—Safety study on the following combination therapies in patients with tumors for which PD-1 inhibitors are indicated, and who have failed PD-1-directed therapy: Antibody B (Q4W)+Antibody A (Q4W)+500 mg (Q4W) INCMGA00012. Q4W doses for Antibody B and Antibody B are based on pharmacokinetics (PK) and receptor occupancy (RO) data from the monotherapy Q3W evaluation of Antibody B and Antibody B in Phase 1 Part 3, above.

(2) Phase 2 will determine preliminary efficacy for the combination of Antibody B+Antibody A+INCMGA00012, and will be completed in two cohorts.

(2a) Phase 2, Cohort A—A population of patients with unresectable/metastatic or adjuvant-treated melanoma (n=15) who have progressed or relapsed on or after prior anti-PDL-1 therapy are being given the combination of 350 mg (Q2W) Antibody B+400 mg (Q2W) Antibody A+500 mg (Q4W) INCMGA00012. If a sufficient number of positive responses are seen (≥1/15 responses), the population will be expanded to include additional participants (+n=15) in a Simon 2-stage expansion to further evaluate safety and efficacy.

(2b) Phase 2, Cohort B—A population of patients with untreated Advanced Melanoma (1 L), and which may have failed CPI in adjuvant (N˜34) are randomized and administered one of the following combination therapies: (2b.i) (N˜17) 450 mg (Q3W) Antibody B+500 mg (Q3W) Antibody A+375 mg (Q3W) INCMGA00012; or (2b.ii) (N˜17) 750 mg (Q3W) Antibody B+1000 mg (Q3W) Antibody A+375 mg (Q3W) INCMGA00012. If a sufficient number of positive responses are seen, the population will be expanded to include additional participants (+n=18 for each cohort) in an expansion to further evaluate safety and efficacy.

The anti-PD-1 antibody for the study is INCMGA00012 (i.e., retifanlimab). The anti-TIM-3 antibody for the study is Antibody A. The anti-LAG-3 antibody for the study is Antibody B. The order of administration of study drugs is: (1) Antibody B, followed by (2) Antibody A, followed by (3) INCMGA00012.

a. Dose Selection for INCMGA00012

INCMGA00012 (i.e., retifanlimab) is administered at 500 mg, once every four weeks (i.e., Q4W), by IV infusion over about 30 minutes on Day 1 of each 28-day cycle. The dosage formulation includes 25 mg/mL in liquid formulation for a 500 mg dose.

The selection of this dose was based on modeling of clinical PK data from a first-in-human monotherapy study, in which 37 participants were treated with doses of 1 mg/kg Q2W, 3 mg/kg Q2W, 3 mg/kg Q4W, 10 mg/kg Q2W, and 10 mg/kg Q4W. Pharmacokinetic data were obtained from 15 participants who received INCMGA00012 500 mg Q4W. The observed AUC_{0-∞} for 500 mg Q4W was close to the steady-state AUC₀₋ₜ based on the population PK analysis of weight-based doses, as is the estimated clearance. The 500 mg Q4W dose had approximately 58% probability to obtain a steady-state trough plasma concentration≥21 μg/mL, which is associated with maximum target engagement and greatest probability of efficacy. Based on these observations, 500 mg Q4W was chosen as the dose regimen.

Additionally, INCMGA00012 is also being studied at 375 mg, once every three weeks (i.e., Q3W), by IV infusion. Without wishing to be bound by any theory, this dose is selected because it is projected that 375 mg Q3W dosing will achieve the same steady-state trough concentration as 500 mg Q4W dosing.

b. Dose Selection for Antibody B

Antibody B is to be administered at 350 mg, once every two weeks (i.e., Q2W), by IV infusion over about 30 minutes on Day 1 of each 14-day cycle. The dosage formulation includes 50 mg/mL in liquid formulation for a 350 mg dose. Antibody B is in a liquid form in sodium acetate, trehalose, polysorbate 80, pH 5.5 with a target protein concentration of 50 mg/mL to be used for IV infusion.

The selection of this dose was based on modeling of clinical PK data from a human monotherapy study, in which 22 participants were treated with IV doses of 25, 75, 250, 350, and 750 mg Q2W in an open-label, nonrandomized, dose escalation, and cohort-expansion study. All doses were well tolerated. Antibody B PK was linear after the first dose and showed moderate to high inter-individual variability. At a dose of 350 mg Q2W (N=3), the steady-state AUC was 30,200 μg/mL·h. The dose of 350 mg Q2W was chosen based on data showing that: (i) receptors on the surface of the cells were fully occupied at trough concentration of post 350 mg Q2W dosing, and (ii) the dose is pharmacologically active, as measured by an increase in peripheral T-cell activation observed in doses greater than 250 mg.

If safety of doses tested in the combination therapy study cannot be established, the dose level for Antibody B will be dropped 1 level from doses tested in the monotherapy study to 250 mg Q2W.

Additionally, Antibody B is also being studied at 450 mg and at 750 mg, once every three weeks (i.e., Q3W), by IV infusion.

c. Dose Selection for Antibody A

Antibody A is to be administered at 400 mg, once every two weeks (i.e., Q2W), by IV infusion over about 30 minutes on Day 1 of each 14-day cycle. The dosage formulation includes 50 mg/mL in liquid formulation for a 400 mg dose. Antibody A is in a liquid form in sodium citrate, sucrose, arginine, polysorbate 80, pH 6.0 with a target protein concentration of 50 mg/mL to be used for IV infusion.

The selection of this dose was based on modeling of clinical PK data from a human monotherapy study, in which 40 participants were treated with IV doses of 10, 30, 100, 200, 400, 800, and 1600 mg Q2W in the open-label, nonrandomized, dose-escalation, and cohort expansion study. All doses were well tolerated. Antibody A showed supra-proportional PK after first dose from 30 mg to 800 mg Q2W and low inter-individual variability on PK exposures. At a dose of 400 mg Q2W, the mean steady-state AUC determined in 4 participants was 43,400 μg/mL·h. The dose of 400 mg was chosen based on data showing that TIM-3 receptor was fully occupied at trough concentrations on the surface of circulating monocytes post 400 mg Q2W dosing.

If safety of doses tested in the combination therapy study cannot be established, the dose level for Antibody A will be dropped 1 level from doses tested in the monotherapy study to 200 mg Q2W.

Additionally, Antibody A is also being studied at 500 mg and at 1000 mg, once every three weeks (i.e., Q3W), by IV infusion.

d. Patient Selection

Patient inclusion criteria includes: (i) Men or women aged 18 years or older; (ii) Phase 1—Participants with locally advanced or metastatic solid tumors for which a PD-1 inhibitor is indicated (locally advanced disease must not be amenable to resection with curative intent) that have failed a PD-1/PD-L1 inhibitor therapy as defined as follows: Subjects must have received prior treatment with anti-PD-1 or anti-PD-L1 therapy (alone or as part of a combination therapy) in the advanced or metastatic setting and: (ii.a) Have PD as their best response to treatment that is confirmed at least 4 weeks later (no less than 28 days), (ii.b) Must have received at least 2 doses of a prior anti-PD-1 or anti-PD-L1 agent, (ii.c) Progressive disease must also be at least 12 weeks from first dose of anti-PD-1 or anti-PD-L1 therapy and confirmed 4 weeks (no less than 28 days) later, and (ii.d) PD should be based on imaging done at least 4 weeks apart; (iii) Phase 2—(iii.a) Participants with histologically confirmed unresectable/metastatic melanoma, whose disease failed prior anti-PD-L1 therapy (alone or as part of a combination therapy) and meeting one of the following criteria: (iii.a1) Participant who failed prior adjuvant anti-PD-L1 therapy for resectable melanoma must have received prior anti-PD-L1 for ≥6 weeks and experienced disease progression while still on active adjuvant therapy containing anti-PD-L1, or participant who had early relapse occurring <24 weeks after end of adjuvant anti-PD-L1 therapy [Progressive disease must be ascertained by confirmatory biopsy collected at baseline], or (iii.a2) Participant whose unresectable/metastatic disease progressed while on or within <24 weeks of completion of anti-PD-L1 as most recent therapy for unresectable/metastatic melanoma [Progressive disease must have been confirmed by imaging≥4 weeks after evidence of initial disease progression]; (iii.b) Participant must have received no more than 2 prior lines of therapy for melanoma and most recent prior therapy must have contained anti-PD-L1 therapy (alone or as part of a combination therapy) either in the adjuvant and/or advanced/metastatic setting [Subjects who have tumors that are BRAF mutant may have received up to 2 prior lines of therapy for melanoma on top of prior BRAF directed therapies] [Participant may have received anti-PD-L1 therapy both in adjuvant and advanced/metastatic setting]; (iii.c) For participants with BRAF-mutant tumors, treatment with a targeted therapy is required and participants must have progressed or been intolerant to the targeted therapy to be eligible; (iii.d) Participants must have fresh biopsy available after completing prior PD-(L)1 therapy or be willing and able to safely undergo pretreatment tumor biopsies (core or excisional); (iii.e) Participant must have at least 1 measurable tumor lesion per RECIST v1.1 [Lesions to be used as measurable disease for the purpose of response assessment must not be lesion used for biopsy and must either (iii.e1) not reside in a field that has been subjected to prior radiotherapy, or (iii.e2) have demonstrated clear evidence of radiographic progression since the completion of prior radiotherapy or biopsy and prior to study enrollment; and (iv) ECOG performance status of 0 or 1.

Patient exclusion criteria includes: (i) Participants with failed laboratory screenings, including (i.a) platelets—<100×10⁹/L, (i.b) Hemoglobin—<9 g/dL, (i.c) ANC—<1.5×109/L, (i.d) ALT—>1.5×ULN (Phase 1) or >2.5×ULN (Phase 2), (i.e) AST—>1.5×ULN (Phase 1) or >2.5×ULN (Phase 2), (i.f) Total bilirubin—≥1.5×ULN, unless conjugated bilirubin≤ULN (conjugated bilirubin only needs to be tested if total bilirubin exceeds ULN) [If there is no institutional ULN, then direct bilirubin must be <40% of total bilirubin; In no case can the total bilirubin exceed 3×ULN]; (i.g) Lactate dehydrogenase—>3×ULN in the absence of hemolysis; (i.h) Calculated creatinine clearance—<50 mL/min [CrCl calculated by Cockcroft-Gault equation]; (i.i) International normalized ratio or prothrombin time—>1.5×ULN; and (i.j) Activated partial thromboplastin time—>1.5×ULN; (ii) Known hypersensitivity or severe reaction to any component of the study drugs or formulation components for INCMGA00012 or Antibody B or Antibody A; (iii) Phase 1—Receipt of anticancer medications or investigational drugs within the following intervals before the first administration of study treatment: (iii.a) ≤14 days for chemotherapy, targeted small molecule therapy, or radiation therapy [Participants must also not require chronic use of corticosteroids and must not have had radiation pneumonitis as a result of treatment]; a 1-week washout is permitted for palliative radiation to non-CNS disease with medical monitor approval; Bisphosphonates and denosumab are permitted concomitant medications; (iii.b) ≤14 days and resolution of all associated toxicities for prior immunotherapy or persistence of active cellular therapy (i.e., chimeric antigen receptor T-cell therapy); (iii.c) <14 days for prior PD-1 pathway-targeted agents; (iii.d) ≤28 days for a prior mAb used for anticancer therapy with the exception of PD-1 pathway-targeted agents and denosumab; (iii.e) ≤7 days for immune-suppressive-based treatment for any reason [Use of inhaled or topical steroids or corticosteroid use for radiographic procedures is permitted]; The use of physiologic corticosteroid replacement therapy may be approved after consultation with the medical monitor; (iii.f) ≤28 days or 5 half-lives (whichever is longer) before the first dose for all other investigational agents or devices [For investigational agents with long half-lives (e.g., >5 days), enrollment before the fifth half-life requires medical monitor approval]; (iii.g) Administration of colony-stimulating factors (including granulocyte colony-stimulating factor, granulocyte macrophage colony-stimulating factor, or recombinant erythropoietin) within 14 days before study Day 1; (iii.h) Has not recovered to ≤Grade 1 from toxic effects of prior therapy (including prior immunotherapy) and/or complications from prior surgical intervention before starting therapy [Participants with stable chronic AEs (≤Grade 2) not expected to resolve (such as neuropathy and alopecia) are exceptions and may enroll; Participants with a history of any grade immune-related ocular AE will be excluded; Participants with a history of a Grade 3 or higher AE of interest from prior immunotherapies are excluded from the dose escalation (Part 1) portion of the study; participants with a history of Grade 3 or higher AE of noninterest may be allowed with medical monitor approval]; (iv) Phase 2—(iv.a) Participant who has discontinued anti-PD-L1 therapy due to toxicity or other reasons unrelated to toxicity, then subsequently experienced disease progression; (iv.b) Participant who had experienced objective response (PR/CR) and had stopped anti-PD-L1 therapy due to maximal benefit; (iv.c) Participant with multiple metastases that achieved mixed tumor response to prior anti-PD-L1 therapy (such as isolated progressive lesion in a context of PR/CR or SD for other lesions) or achieved overall disease progression based only on a single new lesion; (iv.d) Has disease eligible for potentially curative treatment such as surgical resection [Single resection of metastatic lesion in the context of disease progression on prior therapy is not considered curative]; (iv.e) Receipt of anticancer therapy (immunotherapy, chemotherapy, targeted therapy, or hormonal therapy) within 21 days of the first administration of study treatment, with the exception of localized radiotherapy; (iv.f) Palliative radiation therapy administered within 1 week of first dose of study treatment or radiation therapy in the thoracic region that is >30 Gy within 6 months of the first dose of study treatment [Participants must have recovered from all radiation-related toxicities, not require corticosteroids for this purpose, and not have had radiation pneumonitis]; (iv.g) If participant received major surgery, then they must have recovered adequately from toxicities and/or complications from the intervention before starting study treatment; (iv.h) Treatment-related toxicity related to prior therapy that has not recovered to ≤Grade 1 (with the exception of alopecia and anemia not requiring transfusional support), unless approved by the medical monitor; (iv.i) History of immune-related toxicity during prior checkpoint inhibitor therapy for which permanent discontinuation of therapy is recommended (per product label or consensus guidelines), OR any immune-related toxicity requiring intensive or prolonged immunosuppression to manage (with the exception of endocrinopathy that is well-controlled on stable dose of replacement hormones such as hypothyroidism or adrenal insufficiency, or Grade 3 rashes that resolved with topical therapy or asymptomatic lipase elevations that do not require treatment interruption or uveitis that resolved with steroid drops); (v) Has an active autoimmune disease requiring systemic immunosuppression with corticosteroids (>10 mg/day of prednisone or equivalent) or immunosuppressive drugs within 14 days before the first dose of study treatment; (vi) Receiving chronic systemic corticosteroids (>10 mg/day of prednisone or equivalent) [Physiologic corticosteroid replacement therapy at doses>10 mg daily of prednisone or equivalent for adrenal or pituitary insufficiency and in the absence of active autoimmune disease is permitted; Participants with a condition that requires intermittent use bronchodilators, inhaled steroids, or local steroid injections may be admitted (e.g., asthma or chronic obstructive pulmonary disease exacerbation); Participants using topical, ocular, intra-articular, or intranasal steroids (with minimal systemic absorption) may be admitted; Brief course of corticosteroids for prophylaxis (e.g., contrast dye allergy) or study treatment-related standard premedication is permitted]; (vii) Active infections requiring systemic antibiotics, or antifungal or antiviral treatment within 7 days before first dose of study treatment [A participant should be excluded if they have a positive screening test result for SARS-CoV2 infection until test normalization and clinical recovery]; (viii) History of organ transplant, including allogeneic stem cell transplantation; (ix) Evidence of interstitial lung disease or active, noninfectious pneumonitis; (x) Known active HBV or HCV infection or risk of reactivation of HBV or HCV: (x.a) Active hepatitis B infection is defined by positive HBsAg and positive total anti-HBc results; When HBsAg is negative AND HBcAb and/or HBsAb is positive, HBV-DNA should be measured; When HBV-DNA is negative, this participant could be enrolled with close monitoring of HBV activities; (x.b) Active hepatitis C is defined by a positive hepatitis C antibody result and quantitative HCV-RNA results greater than the lower limits of detection of the assay [Participants positive for HCV antibody will be eligible if they are negative for HCV-RNA; Participants who have had definitive treatment for HCV are permitted if HCV RNA is undetectable]; (xi) Participants who are known to be HIV-positive; (xii) Known active brain or CNS metastases, including carcinomatous meningitis [Participants who have previously treated and clinically stable brain or CNS metastases (without evidence of progression by imaging for at least 4 weeks before the first dose of study drug and any neurologic symptoms have returned to baseline), have no evidence of new or enlarging brain metastases or CNS edema and have not required steroids for at least 7 days before the study treatment period are eligible]; (xiii) Known additional malignancy that is progressing or requires active treatment, or history of other malignancy within 2 years of study entry with the exception of cured basal cell or squamous cell carcinoma of the skin, superficial bladder cancer, carcinoma in situ of the cervix, or other noninvasive or indolent malignancy after treatment with curative intent; (xiv) Participants with impaired cardiac function or clinically significant cardiac disease: (xiv.a) New York Heart Association Class III or IV cardiac disease, including preexisting clinically significant ventricular arrhythmia, congestive heart failure, or cardiomyopathy, (xiv.b) Unstable angina pectoris. Acute myocardial infarction≤6 months before study participation, (xiv.c) Other clinically significant heart disease (e.g., ≥Grade 3 hypertension); (xv) History or presence of an abnormal ECG that, in the investigator's opinion, is clinically meaningful [Average QTc interval>460 milliseconds is excluded (corrected by Fridericia or Bazett formula)]; (xvi) Women who are pregnant or breastfeeding; (xvii) Receipt of a live vaccine within 30 days of planned start of study treatment [Examples of live vaccines include measles, mumps, rubella, chicken pox/zoster, yellow fever, rabies, Bacillus Calmette-Guerin, and typhoid vaccine; Seasonal influenza vaccines for injection are generally killed virus vaccines and are allowed; however, intranasal influenza vaccines are live-attenuated vaccines and are not allowed]; (xviii) Any condition that would, in the investigator's judgment, interfere with full participation in the study, including administration of study treatment and attending required study visits; pose a significant risk to the participant; or interfere with interpretation of study data.

e. Study Objectives and Endpoints

Primary, secondary, and exploratory objectives and endpoints for Phase 1 and Phase 2 include the following (listed in Table 1):

TABLE 1
Objectives and Endpoints

| | |
|---|---|
| OBJECTIVES | ENDPOINTS |

PRIMARY
Phase 1

| | |
|---|---|
| To determine recommended Phase 2 dose | Safety and tolerability as assessed by monitoring |
| (RP2D) for all study drugs, based on the safety, | the frequency and severity of adverse events |
| tolerability, and dose-limiting toxicity (DLT), to | (AEs). |
| be used in the following combinations: | |
| Antibody B + Antibody A and Antibody B + | |
| Antibody A + INCMGA00012. | |

Phase 2

| | |
|---|---|
| To determine the safety of Antibody B + | Safety and tolerability as assessed by monitoring |
| Antibody A + INCMGA00012 in participants | the frequency and severity of adverse events |
| with melanoma who have progressed or relapsed | (AEs). |
| on or after anti-PD-L1 therapy as their most | |
| recent line of therapy. | |
| To determine the preliminary efficacy of | Objective response rate (ORR), defined as the |
| Antibody B + Antibody A + INCMGA00012 in | percentage of participants having a complete |
| participants with melanoma who have progressed | response (CR) or partial response (PR), as |
| or relapsed on or after anti-PD-L1 therapy as | determined by assessment of radiographic |
| their most recent line of therapy. | disease assessments per RECIST v1.1. |
| | Duration of response (DOR), defined as the |
| | time from earliest date of disease response (CR |
| | or PR) until earliest date of disease progression, |
| | as determined by investigator assessment of |
| | radiographic disease per RECIST v1.1, or death |
| | from any cause, if occurring sooner than |
| | progression. |
| | Disease control rate (DCR), defined as |
| | percentage of participants having CR, PR, or |
| | stable disease (SD) as best on-study response. |

SECONDARY
Phase 1

| | |
|---|---|
| To determine the preliminary efficacy of the | ORR, defined as the percentage of participants |
| combinations of Antibody B + Antibody A and | having a CR or PR, will be determined by |
| of Antibody B + Antibody A + INCMGA00012 | investigator assessment of radiographic disease |
| in participants with solid tumors who have failed | assessments per RECIST v1.1. |
| anti-PD-1 therapy. | Progression-free survival (PFS), defined as the |
| | time from date of first dose of study treatment |
| | until the earliest date of disease progression, as |
| | determined by assessment of objective |
| | radiographic disease per RECIST v1.1 |

EXPLORATORY
Phase 1 and Phase 2

| | |
|---|---|
| To determine the PK of Antibody B, Antibody | PK of Antibody B, Antibody A, and |
| A and INCMGA00012 when | INCMGA00012, including Cₘₐₓ, Tₘₐₓ, Cₘᵢₙ, and |
| given in combination. | AUCₜ. |
| To evaluate immunogenicity of Antibody B, | Immunogenicity, defined as the occurrence of |
| Antibody A, and INCMGA00012 when given in | specific antidrug antibody (ADA) to the |
| combination. | antibodies being tested. |
| To explore tumor and immune cell infiltrate | Biomarker effects will be assessed, including |
| biomarkers that may predict the pharmacologic | (a) Whole blood immune cell population profile, |
| activity of the combinations of immuno- | (b) Plasma markers of inflammation or immune |
| oncology drugs being tested. | modulation, (c) Monitoring of changes in RNA |
| | expression, (d) Tumor biopsy analysis to |
| | evaluate expression of PD-1, LAG-3, TIM-3, and |
| | their ligands and immune cell infiltrate in tumor |
| | tissue and correlate expression with efficacy. |
| To evaluate the efficacy of Antibody B + | ORR, DOR, and PFS will be determined by |
| Antibody A + INCMGA00012 using | assessment of radiographic disease assessments |
| iRECIST. | per iRECIST criteria. EQ-5D and EORTC QLQ- |
| | C30 will be assessed throughout the study. |

EXPLORATORY
Phase 2 Only

| | |
|---|---|
| To determine the preliminary efficacy of | ORR, defined as the percentage of participants |
| Antibody B + Antibody A + INCMGA00012 in | having a CR or PR, as determined by assessment |
| participants with LAG-3- positive (≥5%) | of radiographic disease assessments per RECIST |
| melanoma who have progressed or relapsed on or | v1.1. |
| after anti-PD-L1 therapy as their most recent line | DOR, defined as the time from earliest date of |
| of therapy. | disease response (CR or PR) until earliest date of |
| | disease progression, as determined by |
| | investigator assessment of radiographic disease |
| | per RECIST v1.1, or death from any cause, if |
| | occurring sooner than progression. |
| | DCR, defined as percentage of participants |
| | having CR, PR, or SD as best on-study response. |
| To compare the efficacy of Antibody B + | ORR, DOR, and PFS will be determined by |
| Antibody A + INCMGA00012 in participants | assessment of radiographic disease assessments |
| with melanoma with primary versus secondary | per iRECIST criteria. |
| resistance to prior anti- PD-(L)1 therapy (shall | |
| collected data allow such analysis). | |
| Determine the effect of combination treatment on | EQ-5D and EORTC QLQ-C30 will be assessed |
| health outcomes and quality of life of the | throughout the study. |
| participants. | |

f. Study Results

Combination regimens were generally well tolerated with a safety profile consistent with that of the immune checkpoint inhibitor class. No novel toxicities were observed. Immune related TEAEs were reported in 2 patients on the triplet regimen (1 with grade 3 myocarditis and pericardial effusion, and 1 with infusion related reaction), and none on the doublet regimen; no grade≥3 infusion related adverse events were reported with either regimen.

Overall Antitumor Activity Response (per RECIST v1.1) is summarized in Table 2 [Disease control was defined as patients having complete response, partial response, or stable disease on or after day 42]:

TABLE 2
Overall Antitumor Activity

| | PART 1 | | PART 2 | |
|---|---|---|---|---|
| | DOUBLET | | TRIPLET | |
| VARIABLE | (n = 10) | | (n = 11) | |
| Objective response rate (95% CI), % | 0 | (0.0-30.8) | 9.1 | (0.2-41.3) |

Best overall response, n (%)

| | | | | |
|---|---|---|---|---|
| Partial Response (PR) | 0 | (0.0) | 1 | (9.1) |
| Stable Disease (SD) | 3 | (30.0) | 3 | (27.3) |
| Progressive Disease (PD) | 6 | (60.0) | 5 | (45.5) |
| Not assessed/No data available | 1 | (10.0) | 2 | (18.2) |
| Disease control, n (%) | 3 | (30.0) | 4 | (36.4) |

The objective response rate was 4.8%. The best overall response in part 1 was stable disease, and in part 2 was a confirmed and durable partial response in one patient with checkpoint therapy refractory melanoma (failed both pembrolizumab in adjuvant and ipilimumab plus nivolumab in first line settings). This patient completed 2 years of triplet therapy and was ongoing with a sustained response at the data cutoff in the first follow up assessment.

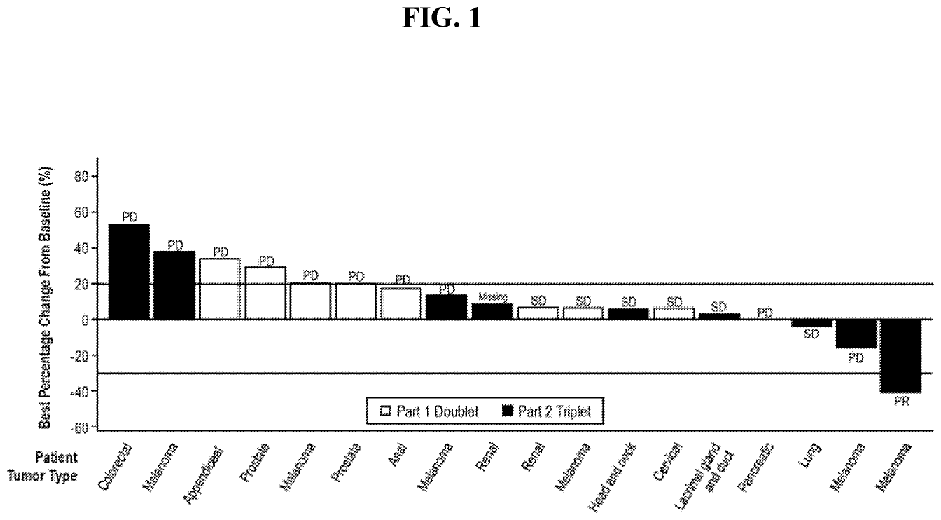

The best percentage change from baseline in target lesion size (sum of diameters) among individual patients assessable for response (n=21) is shown in FIG. 1. The Upper Limit line indicates a criterion for PD (≥20% increase in sum of target lesion diameters) and the lower limit line indicates a criterion for PR (≥30% decrease in sum of target lesion diameters).

### Example 3. Combination Therapy Study of Anti-PD-1 Active Agent, Anti-TIM-3 Active Agent, and Anti-LAG-3 Active Agent in Squamous Cell Carcinoma of the Head and Neck (SCCHN)

A study is being conducted to evaluate the efficacy of the combination of an anti-PD-1 active agent, an anti-TIM-3 active agent, and/or an anti-LAG-3 active agent in participants with squamous cell carcinoma of the head and neck (SCCHN). A study population of about 164 patients is being selected with treatment-naive recurrent or metastatic PD-L1+ SCCHN.

The study includes three patient cohorts of about 54 patients each, being randomized 1:1:1 to receive one of three treatments intravenously for up to 2 years: (Group 1)—INCMGA00012 monotherapy at 500 mg every 4 weeks (q4w), plus 2 placebo control patients; (Group 2) INCMGA00012 at 500 mg q4w, and Antibody B at 350 mg every 2 weeks (q2w), plus one placebo control patient; and (Group 3) INCMGA00012 at 500 mg q4w, Antibody B at 350 mg (q2w), and Antibody A at 400 mg (q2w). Patient randomization is being stratified by LAG-3 expression status (positive [≥5%] or negative [<5%] tumor proportion score), PD-L1 expression status (combined positive scores 1%-19% vs ≥20%), and human papillomavirus p16 status (positive vs negative; oropharyngeal tumors only).

Patient inclusion criteria includes: (i) ≥18 years of age; (ii) ECOG performance status of 0 or 1; (iii) Recurrent or metastatic SCCHN: (iii.a) that is histologically or cytologically confirmed, (iii.b) has primary tumors in oropharynx, oral cavity, hypopharynx, or larynx, and (iii.c) is not amenable to potentially curative surgery or radiation therapy; (iv) a PD-L1-positive tumor status of 11% combined positive score determined at a central laboratory; and (v) ≥1 measurable tumor lesion per RECIST v1.1, wherein the tumor for measurement either (a) must not have been biopsied or irradiated or (b) must have shown clear evidence of progression since biopsy/radiotherapy.

Patient exclusion criteria includes: (i) Prior systemic therapy for metastatic or recurrent SCCHN; (ii) Progressive or recurrent disease≤6 months from end of last systemic treatment for locally advanced SCCHN; (iii) SCCHN primary tumors of the nasopharynx, sinonasal cavity, or salivary gland; (iv) Prior PD-(L)1, LAG-3, TIM-3, or other ICI therapies in any treatment setting; (v) Treatment with any other therapy or participation in a clinical study≤21 days before first study dose; (vi) Tumor invasion of major blood vessels with active bleeding; (vii) Less than 3-month life expectancy; (viii) Active autoimmune disease requiring systemic immunosuppression with corticosteroids (>10 mg/day of prednisone or equivalent) or immunosuppressive drugs 2 years of first dose of study treatment; (ix) <4 weeks since radiation therapy of CNS or known active CNS metastases and/or carcinomatous meningitis; and (x) Laboratory values indicative of cytopenia, renal/hepatic impairment, or hypercoagulable state.

Primary, secondary, and safety objectives and endpoints for Phase 1 and Phase 2 include the following: Primary Efficacy—PFS (randomization date to earliest date of disease progression or death); Secondary Efficacy—(i) Objective response (PR+CR); (ii) Duration of response (earliest date of PR or CR until earliest date of disease progression or death); (iii) Disease control (CR+PR+SD†); and (iv) OS (randomization date until death from any cause); and Safety—AE (physical examinations, changes in vital signs, blood chemistry) and AE leading to study interruption, reduction, or discontinuation. Radiologic tumor assessments (tomography or magnetic resonance imaging) will be performed every 8 weeks.

### Example 4. Combination Therapy Study of Anti-PD-1 Active Agent, Anti-TIM-3 Active Agent, and Anti-LAG-3 Active Agent in MSI-H Advanced or Metastatic Endometrial Cancer

A study is being conducted to evaluate the efficacy of the combination of an anti-PD-1 active agent, an anti-TIM-3 active agent, and/or an anti-LAG-3 active agent in participants who have MSI-H advanced or metastatic endometrial cancer with evidence of disease progression on or following platinum-based chemotherapy and previous PD-L1 inhibitory therapy. A study population of up to about 40 female participants (at least 18 years of age) is being selected.

The study includes each patient receiving the following treatment intravenously (over 30 minutes) for up to 2 years: INCMGA00012 at 500 mg q4w, Antibody B at 350 mg (q2w), and Antibody A at 400 mg (q2w).

Patient inclusion criteria includes: (i) Women≥18 years of age; (ii) Histologically confirmed diagnosis of advanced or metastatic endometrial cancer with disease progression on or after treatment with at least 1 platinum-containing regimen for advanced or metastatic disease; (iii) Radiological evidence of disease progression on or after prior PD-(L)1 therapy—(iii.a) Participant must have received at least 2 doses of prior PD-(L)1 therapy, (iii.b) Participant may have received PD-(L)1 therapy either alone or in combination, (iii.c) Disease progression must have occurred on or after the first on-treatment scan and must have been confirmed subsequently by imaging≥4 weeks after evidence of initial disease progression. Note: baseline scan within the study may serve as a confirmatory scan for progressive disease, (iii.d) Participant may have achieved objective response (CR or PR) to prior PD-L1 therapy followed by disease recurrence; (iv) Tumor tissue tested as MSI-H centrally or locally using PCR assay; (v) Must have at least 1 measurable tumor lesion per RECIST v1.1; and (vi) ECOG performance status 0 to 1.

Patient exclusion criteria includes: (i) Histologically confirmed diagnosis of sarcoma of the uterus; (ii) Has disease eligible for potentially curative treatment with standard chemotherapy, surgical resection, or chemoradiotherapy; (iii) Receipt of anticancer therapy within 28 days of the first administration of study treatment, with the exception of localized radiotherapy; (iv) Toxicity of prior therapy that has not recovered to ≤Grade 1 (with the exception of alopecia and anemia not requiring transfusional support), unless approved by the medical monitor; (v) Immune-related toxicity during prior checkpoint inhibitor therapy for which permanent discontinuation of therapy is recommended (per product label or consensus guidelines), or severe immune-related toxicity requiring intensive (eg, use of infliximab) or prolonged immunosuppression (eg, >6 weeks) to manage (with the exception of endocrinopathy that is well-controlled on replacement hormones); (vi) Previous treatment with LAG-3 or TIM-3 directed therapy or lenvatinib; (vii) Participants with multiple metastases that achieved mixed tumor response to prior anti-PD-(L)1 therapy (such as isolated progressive lesion in a context of PR/CR or SD for other lesions) or achieved overall disease progression based only on a single new lesion; (viii) Exclusionary Laboratory Values—Platelets [<100×109/L], Hemoglobin [<9 g/dL], ANC [<1.5×109/L], ALT [>2×ULN or >5×ULN for participants with liver metastases], AST [>2×ULN or >5×ULN for participants with liver metastases], Total bilirubin [≥1.5×ULN unless conjugated bilirubin≤ULN (conjugated bilirubin only needs to be tested if total bilirubin exceeds ULN), if there is no institutional ULN, then direct bilirubin must be <40% of total bilirubin], CrCl [<30 mL/min calculated by Cockcroft-Gault equation (glomerular filtration rate can also be used in place of CrCl)], INR or PT [>1.5×ULN unless on therapeutic anticoagulants], aPTT [>1.5×ULN], cardiac function [>2×institutional ULN]; (ix) Has an active autoimmune disease requiring systemic immunosuppression with corticosteroids (>10 mg/day of prednisone or equivalent) or immunosuppressive drugs within 14 days before the first dose of study treatment; (x) Receiving chronic systemic corticosteroids (>10 mg/day of prednisone or equivalent); (xi) Active infections requiring systemic antibiotics, or antifungal or antiviral treatment; (xii) History of organ transplant, including allogeneic stem cell transplantation; (xiii) Receiving probiotics as of the first dose of study treatment; (xiv) Known active CNS metastases and/or carcinomatous meningitis; (xv) Known additional malignancy that is progressing or requires active treatment, or history of other malignancy within 2 years of study entry with the exception of cured basal cell or squamous cell carcinoma of the skin, superficial bladder cancer, carcinoma in situ of the cervix, or other noninvasive or indolent malignancy, or cancers from which the participant has been disease-free for >1 year, after treatment with curative intent; (xvi) Has known active hepatitis B or C (positive HBsAg and positive total anti-HBc results) or HIV, HBV, HCV, or HDV coinfection; (xvii) Known hypersensitivity to any of the study drugs, excipients, or another monoclonal antibody that cannot be controlled with standard measures (e.g., antihistamines and corticosteroids); (xviii) Participants with impaired cardiac function or clinically significant cardiac disease; (xix) Women who are pregnant or breast-feeding; (xx) If participant received major surgery, then they must have recovered adequately from toxicities and/or complications from the intervention before starting study treatment; (xxi) Has received a live vaccine within 28 days of the planned start of study treatment; (xxii) Evidence of interstitial lung disease or active, noninfectious pneumonitis; (xxiii) Current use of prohibited medication; (xxiv) History of a gastrointestinal condition (e.g., inflammatory bowel disease, Crohn's disease, ulcerative colitis) that may affect oral drug absorption.

Primary, secondary, and safety objectives and endpoints include the following: (i) overall response rate (ORR), defined as the proportion of participants having a complete response (CR) or partial response (PR) according to RECIST v1.1 (as determined by the investigator); (ii) To evaluate the safety and tolerability of INCMGA00012 in combination; (iii) to assess additional measures of clinical activity for INCMGA00012 combinations—(iii.a) DOR may be provided if a sufficient number of responders are observed in an individual treatment group, (iii.b) DCR, defined as the proportion of participants with CR, PR, or SD (as determined by investigator) as best response, (iii.c) PFS, defined as the time from the first dose of study treatment until disease progression (as defined by the investigator) or death due to any cause, (iii.d) OS, defined as the time from the first dose of study treatment until death due to any cause; (iv) To evaluate the PK of INCMGA00012 when given in combination with other therapies (including Cₘₐₓ, Tₘₐₓ, Cₘᵢₙ, and AUC₀₋ₜ); (v) To evaluate the PK of each of the other study drugs given in combination with INCMGA00012 (including Cₘₐₓ, Tₘₐₓ, Cₘᵢₙ, and AUC₀₋ₜ); (vi) To evaluate the immunogenicity of INCMGA00012 when given in combination with Antibody B and Antibody A (proportion of participants who develop anti-drug antibodies (ADAs)); (vii) To evaluate the relationship between baseline and treatment biomarkers of clinical activity (Association of blood or tumor analytes, immune cell profile and other relevant marker levels from baseline with treatment outcomes).

1. A method for treating cancer in a human subject in need thereof, the method comprising administering to the subject at least two of: (i) about 375-500 mg of an anti-PD-1 active agent once every three weeks or four weeks; (ii) about 400-1000 mg of an anti-TIM-3 active agent once every two weeks, three weeks, or four weeks; and (iii) about 350-750 mg of an anti-LAG-3 active agent once every two weeks, three weeks, or four weeks.

2. The method of claim 1, wherein the method comprises administering to the subject at least two of: (i) 500 mg of an anti-PD-1 active agent once every four weeks; (ii) 400 mg of an anti-TIM-3 active agent once every two weeks; and (iii) 350 mg of an anti-LAG-3 active agent once every two weeks.

3. The method of claim 1, wherein the method comprises administering to the subject at least two of: (i) 375 mg of an anti-PD-1 active agent once every three weeks; (ii) 500 mg of an anti-TIM-3 active agent once every three weeks; and (iii) 450 mg of an anti-LAG-3 active agent once every three weeks.

4. The method of claim 1, wherein the method comprises administering to the subject at least two of: (i) 375 mg of an anti-PD-1 active agent once every three weeks; (ii) 1000 mg of an anti-TIM-3 active agent once every three weeks; and (iii) 750 mg of an anti-LAG-3 active agent once every three weeks.

5. (canceled)

6. A combination therapy comprising at least two of: (i) about 375-500 mg of an anti-PD-1 active agent; (ii) about 400-1000 mg of an anti-TIM-3 active agent; and (iii) about 350-750 mg of an anti-LAG-3 active agent.

7-33. (canceled)

34. A kit for treating a cancer in a human subject in need thereof, the kit comprising at least two of: (i) about 375-500 mg of an anti-PD-1 active agent; about (ii) 400-1000 mg of an anti-TIM-3 active agent; and (iii) about 350-750 mg of an anti-LAG-3 active agent; and instructions to administer the anti-PD-1 active agent once every three weeks or four weeks and to administer the anti-TIM-3 and anti-LAG-3 active agents once every two weeks, three weeks, or four weeks.

35-38. (canceled)

39. The method of claim 1, wherein the anti-PD-1 active agent comprises an anti-PD-1 antibody, or PD-1 binding-fragment thereof, which comprises:
(i) a Heavy Chain Variable Domain (VH), comprising a CDRH1 Domain having the amino acid sequence SYWMN (SEQ ID NO: 1), a CDRH2 Domain having the amino acid sequence VIHPSDSETWLDQKFK (SEQ ID NO: 2), and a CDRH3 Domain having the amino acid sequence EHYGTSPFAY (SEQ ID NO: 3); and
(ii) a Light Chain Variable Domain (VL), comprising a CDRL1 Domain having the amino acid sequence RASESVDNYGMSFMNW (SEQ ID NO: 4), a CDRL2 Domain having the amino acid sequence AASNQGS (SEQ ID NO: 5), and a CDRL3 Domain having the amino acid sequence QQSKEVPYT (SEQ ID NO: 6).

40. The method of claim 39, wherein the Heavy Chain Variable Domain of the anti-PD-1 antibody or fragment thereof comprises an amino acid sequence which is at least 75%, 80%, 85%, 90%, 95%, 99%, or 100% identical to: QVQLVQSGAEVKKPGASVKVSCKASGYSFTSYWMNWVRQAPGQGLEWIGVIHP SDSETWLDQKFKDRVTITVDKSTSTAYMELSSLRSEDTAVYYCAREHYGTSPFAY WGQGTLVTVSS (SEQ ID NO: 7); and
wherein the Light Chain Variable Domain of the anti-PD-1 antibody or fragment thereof comprises an amino acid sequence which is at least 75%, 80%, 85%, 90%, 95%, 99%, or 100% identical to:
(SEQ ID NO: 8)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIK.

41. (canceled)

42. The method of claim 39, wherein the anti-PD-1 antibody or fragment thereof comprises a Heavy Chain (HC) sequence which is at least 75%, 80%, 85%, 90%, 95%, 99%, or 100% identical to: QVQLVQSGAEVKKPGASVKVSCKASGYSFTSYWMNWVRQAPGQGLEWIGVIHP SDSETWLDQKFKDRVTITVDKSTSTAYMELSSLRSEDTAVYYCAREHYGTSPFAY WGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRV ESKYGPPCPPCPAPEFLGGPSVFLFPPKPKDTLMISRTPEVTCVVVDVSQEDPEV QFNWYVDGVEVHNAKTKPREEQFNSTYRVVSVLTVLHQDWLNGKEYKCKVSNK GLPSSIEKTISKAKGQPREPQVYTLPPSQEEMTKNQVSLTCLVKGFYPSDIAVEW ESNGQPENNYKTTPPVLDSDGSFFLYSRLTVDKSRWQEGNVFSCSVMHEALHN HYTQKSLSLSLG (SEQ ID NO: 9); and
wherein the anti-PD-1 antibody or fragment thereof comprises a Light Chain (LC) sequence which is at least 75%, 80%, 85%, 90%, 95%, 99%, or 100% identical to:
(SEQ ID NO: 10)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

43-45. (canceled)

46. The method of claim 39, wherein the anti-PD-1 antibody is retifanlimab.

47. The method of claim 1, wherein the anti-PD-1 active agent is administered intravenously.

48. The method of claim 1, wherein the anti-TIM-3 active agent comprises an anti-TIM-3 antibody, or TIM-3 binding-fragment thereof, which comprises:
(i) a Heavy Chain Variable Domain (V_{H}), comprising a CDRH1 Domain having the amino acid sequence RQNAWS (SEQ ID NO: 11), a CDRH2 Domain having the amino acid sequence WVSAISGSGGSTY (SEQ ID NO: 12), and a CDRH3 Domain having the amino acid sequence AKGGDYGGNYFD (SEQ ID NO: 13); and
(ii) a Light Chain Variable Domain (V_{L}), comprising a CDRL1 Domain having the amino acid sequence RASQSVSSYLA (SEQ ID NO: 14), a CDRL2 Domain having the amino acid sequence DASNRAT (SEQ ID NO: 15), and a CDRL3 Domain having the amino acid sequence QQYGSSPLT (SEQ ID NO: 16).

49. The method of claim 48, wherein the Heavy Chain Variable Domain of the anti-TIM-3 antibody or fragment thereof comprises an amino acid sequence which is at least 75%, 80%, 85%, 90%, 95%, 99%, or 100% identical to: EVQLVESGGGLVQPGGSLRLSCAASGFTFRQNAWSWVRRAPGKGLEWVSAISG SGGSTYYADSVKGRFTISRDNSKNTLYLQMNSLRAEDTAVYYCAKGGDYGGNYF DYWGQGTLVTVSS (SEQ ID NO: 17); and
wherein the Light Chain Variable Domain of the anti-TIM-3 antibody or fragment thereof comprises an amino acid sequence which is at least 75%, 80%, 85%, 90%, 95%, 99%, or 100% identical to:
(SEQ ID NO: 18)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIK.

50. (canceled)

51. The method of claim 48, wherein the anti-TIM-3 antibody or fragment thereof comprises a Heavy Chain (HC) sequence which is at least 75%, 80%, 85%, 90%, 95%, 99%, or 100% identical to: EVQLVESGGGLVQPGGSLRLSCAASGFTFRQNAWSWVRRAPGKGLEWVSAISG SGGSTYYADSVKGRFTISRDNSKNTLYLQMNSLRAEDTAVYYCAKGGDYGGNYF DYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYASTYRVVSVLTVLHQDWLNGKEYKC KVSNKALPAPIEKTISKAKGQPREPQVYTLPPSREEMTKNQVSLTCLVKGFYPSDI AVEWESNGQPENNYKTTPPVLDSDGSFFLYSKLTVDKSRWQQGNVFSCSVMHE ALHNHYTQKSLSLSPG (SEQ ID NO: 19); and wherein the anti-TIM-3 antibody or fragment thereof comprises a Light Chain (LC) sequence which is at least 75%, 80%, 85%, 90%, 95%, 99%, or 100% identical to:
(SEQ ID NO: 20)
EIVLTQSPATLSLSPGERATLSCRASQSVSSYLAWYQQKPGQAPRLLIY
DASNRATGIPASFSGSGSGTDFTLTISRLEPEDFAVYYCQQYGSSPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

52-56. (canceled)

57. The method of claim 51, wherein the anti-TIM-3 antibody comprises a Heavy Chain (HC) sequence comprising SEQ ID NO: 19 and a Light Chain (LC) sequence comprising SEQ ID NO: 20.

58. The method of claim 1, wherein the anti-TIM-3 active agent is administered intravenously.

59. The method of claim 1, wherein the anti-LAG-3 active agent comprises an anti-LAG-3 antibody, or LAG-3 binding-fragment thereof, which comprises:
i) a Heavy Chain Variable Domain (V_{H}), comprising a CDRH1 Domain having the amino acid sequence DTYIH (SEQ ID NO: 21), a CDRH2 Domain having the amino acid sequence EIDPANDNTKYDPKFQG (SEQ ID NO: 22), and a CDRH3 Domain having the amino acid sequence YYYKYDVGGFDY (SEQ ID NO: 23); and
ii) a Light Chain Variable Domain (V_{L}), comprising a CDRL1 Domain having the amino acid sequence SVSSSISSSNLH (SEQ ID NO: 24), a CDRL2 Domain having the amino acid sequence GTSNLAS (SEQ ID NO: 25), and a CDRL3 Domain having the amino acid sequence QQWSSYPFT (SEQ ID NO: 26).

60. The method of claim 59, wherein the Heavy Chain Variable Domain of the anti-LAG-3 antibody or fragment thereof comprises an amino acid sequence which is at least 75%, 80%, 85%, 90%, 95%, 99%, or 100% identical to: QVQMVQSGAEVKKPGASVKVSCKASGFNIKDTYIHWVRQAPGQGLEWMGEIDP ANDNTKYDPKFQGRVTITADTSTSTVYMELSSLRSEDTAVYYCATYYYKYDVGGF DYWGQGTLVTVSS (SEQ ID NO: 27); and
wherein the Light Chain Variable Domain of the anti-LAG-3 antibody or fragment thereof comprises an amino acid sequence which is at least 75%, 80%, 85%, 90%, 95%, 99%, or 100% identical to:
(SEQ ID NO: 28)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIK.

61. (canceled)

62. The method of claim 59, wherein the anti-LAG-3 antibody or fragment thereof comprises a Heavy Chain (HC) sequence which is at least 75%, 80%, 85%, 90%, 95%, 99%, or 100% identical to: QVQMVQSGAEVKKPGASVKVSCKASGFNIKDTYIHWVRQAPGQGLEWMGEIDP ANDNTKYDPKFQGRVTITADTSTSTVYMELSSLRSEDTAVYYCATYYYKYDVGGF DYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK RVEPKSCDKTHTCPPCPAPELLGGPSVFLFPPKPKDTLMISRTPEVTCVVVDVSH EDPEVKFNWYVDGVEVHNAKTKPREEQYASTYRVVSVLTVLHQDWLNGKEYKC KVSNKALPAPIEKTISKAKGQPREPQVYTLPPSREEMTKNQVSLTCLVKGFYPSDI AVEWESNGQPENNYKTTPPVLDSDGSFFLYSKLTVDKSRWQQGNVFSCSVMHE ALHNHYTQKSLSLSPG (SEQ ID NO: 29); and
wherein the anti-LAG-3 antibody or fragment thereof comprises a Light Chain (LC) sequence which is at least 75%, 80%, 85%, 90%, 95%, 99%, or 100% identical to:
(SEQ ID NO: 30)
EIVLTQSPGTLSLSPGERATLSCSVSSSISSSNLHWYQQKPGQAPRLLI
YGTSNLASGIPDRFSGSGSGTDFTLTISRLEPEDFAVYYCQQWSSYPFT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

63-67. (canceled)

68. The method of claim 62, wherein the anti-LAG-3 antibody comprises a Heavy Chain (HC) sequence comprising SEQ ID NO: 29 and a Light Chain (LC) sequence comprising SEQ ID NO: 30.

69. The method of claim 1, wherein the anti-LAG-3 active agent is administered intravenously.

70. The method of claim 1, wherein the anti-PD-1 active agent is administered before the anti-TIM-3 active agent and the anti-LAG-3 active agent; and optionally wherein (a) the anti-TIM-3 composition is administered before the anti-LAG-3 composition, (b) the anti-LAG-3 composition is administered before the anti-TIM-3 composition, or (c) the anti-TIM-3 composition and the anti-LAG-3 composition are administered concurrently.

71. The method of claim 1, wherein the anti-PD-1 active agent is administered after the anti-TIM-3 active agent and the anti-LAG-3 active agent; and optionally wherein (a) the anti-TIM-3 composition is administered before the anti-LAG-3 composition, (b) the anti-LAG-3 composition is administered before the anti-TIM-3 composition, or (c) the anti-TIM-3 composition and the anti-LAG-3 composition are administered concurrently.

72. The method of claim 1, wherein the anti-PD-1 active agent is administered in a pharmaceutical composition comprising: acetate, sucrose, polysorbate 80 (“PS80”), and water, and has a pH of about 4.0 to about 6.5; optionally wherein the concentration of the anti-PD-1 agent in the pharmaceutical composition is about 10 mg/mL to about 100 mg/mL.

73. The method of claim 1, wherein the anti-TIM-3 active agent is administered in a pharmaceutical composition comprising: sodium citrate, sucrose, arginine, polysorbate 80, and has a pH 6.0; optionally wherein the concentration of the anti-TIM-3 agent in the pharmaceutical composition is about 50 mg/mL.

74. The method of claim 1, wherein the anti-LAG-3 active agent is administered in a pharmaceutical composition comprising: sodium acetate, trehalose, polysorbate 80, and has a pH 5.5; optionally wherein the concentration of the anti-LAG-3 agent in the pharmaceutical composition is about 50 mg/mL.

75. The method of claim 1, wherein the cancer comprises a tumor; optionally wherein the tumor is a locally advanced tumor, a metastatic solid tumor, or a combination thereof.

76. The method of claim 75, wherein the cancer comprises a tumor for which a PD-1 inhibitor is indicated.

77. The method of claim 75, wherein the subject has received prior treatment with at least one anti-PD-1/anti-PDL-1 therapy; optionally wherein the subject has a cancer which failed the prior PD-1/PDL-1 inhibitor therapy; optionally wherein the subject has a cancer which continued to progress during the prior PD-1/PDL-1 inhibitor therapy.

78. The method of claim 75, wherein the tumor has acquired resistance to anti-PD-1 therapy, has innate resistance to anti-PD-1 therapy, or has a combination thereof.

79. The method of claim 75, wherein the tumor has a minimum LAG-3 expression of greater than or equal to 5% of LAG-3-positive immune cells (e.g., lymphocytes and macrophages) relative to all nucleated cells within the tumor region, as shown by immunohistochemical assay.

80. The method of claim 1, wherein the cancer is melanoma; optionally unresectable and/or metastatic melanoma.

81. The method of claim 1, wherein the cancer is squamous cell carcinoma of the head and neck (SCCHN); optionally recurrent or metastatic PD-L1+ SCCHN.

82. The method of claim 1, wherein the cancer is endometrial cancer; optionally MSI-H advanced or metastatic endometrial cancer; optionally with evidence of disease progression on or following platinum-based chemotherapy and previous PD-L1 inhibitory therapy.

83. The method of claim 1, wherein the administration or treatment produces at least one therapeutic effect chosen from: a reduction in size of a tumor, reduction in number of metastasic lesions over time, complete response, partial response, and stable disease, according to RECIST v1.1 criteria.


SEQUENCE LISTING (55 sequences)

SEQ ID NO: 1 - protein, 5 aa, Synthetic construct
SYWMN

SEQ ID NO: 2 - protein, 16 aa, Synthetic construct
VIHPSDSETWLDQKFK

SEQ ID NO: 3 - protein, 10 aa, Synthetic construct
EHYGTSPFAY

SEQ ID NO: 4 - protein, 16 aa, Synthetic construct
RASESVDNYGMSFMNW

SEQ ID NO: 5 - protein, 7 aa, Synthetic construct
AASNQGS

SEQ ID NO: 6 - protein, 9 aa, Synthetic construct
QQSKEVPYT

SEQ ID NO: 7 - protein, 119 aa, Synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 8 - protein, 111 aa, Synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 9 - protein, 445 aa, Synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 218 aa, Synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 6 aa, Synthetic construct
RQNAWS

SEQ ID NO: 12 - protein, 13 aa, Synthetic construct
WVSAISGSGGSTY

SEQ ID NO: 13 - protein, 12 aa, Synthetic construct
AKGGDYGGNYFD

SEQ ID NO: 14 - protein, 11 aa, Synthetic construct
RASQSVSSYLA

SEQ ID NO: 15 - protein, 7 aa, Synthetic construct
DASNRAT

SEQ ID NO: 16 - protein, 9 aa, Synthetic construct
QQYGSSPLT

SEQ ID NO: 17 - protein, 120 aa, Synthetic construct
EVQLVESGGGLVQPGGSLRLSCAASGFTFRQNAWSWVRRAPGKGLEWVSAISGSGGSTYYADSVKGRFTISRDNSKNTLYLQMNSLRAEDTAVYYCAKGGDYGGNYFDYWGQGTLVTVSS

SEQ ID NO: 18 - protein, 107 aa, Synthetic construct
EIVLTQSPATLSLSPGERATLSCRASQSVSSYLAWYQQKPGQAPRLLIYDASNRATGIPASFSGSGSGTDFTLTISRLEPEDFAVYYCQQYGSSPLTFGGGTKVEIK

SEQ ID NO: 19 - protein, 449 aa, Synthetic construct
EVQLVESGGGLVQPGGSLRLSCAASGFTFRQNAWSWVRRAPGKGLEWVSAISGSGGSTYYADSVKGRFTISRDNSKNTLYLQMNSLRAEDTAVYYCAKGGDYGGNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, Synthetic construct
EIVLTQSPATLSLSPGERATLSCRASQSVSSYLAWYQQKPGQAPRLLIYDASNRATGIPASFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, Synthetic construct
DTYIH

SEQ ID NO: 22 - protein, 17 aa, Synthetic construct
EIDPANDNTKYDPKFQG

SEQ ID NO: 23 - protein, 12 aa, Synthetic construct
YYYKYDVGGFDY

SEQ ID NO: 24 - protein, 12 aa, Synthetic construct
SVSSSISSSNLH

SEQ ID NO: 25 - protein, 7 aa, Synthetic construct
GTSNLAS

SEQ ID NO: 26 - protein, 9 aa, Synthetic construct
QQWSSYPFT

SEQ ID NO: 27 - protein, 121 aa, Synthetic construct
QVQMVQSGAEVKKPGASVKVSCKASGFNIKDTYIHWVRQAPGQGLEWMGEIDPANDNTKYDPKFQGRVTITADTSTSTVYMELSSLRSEDTAVYYCATYYYKYDVGGFDYWGQGTLVTVSS

SEQ ID NO: 28 - protein, 108 aa, Synthetic construct
EIVLTQSPGTLSLSPGERATLSCSVSSSISSSNLHWYQQKPGQAPRLLIYGTSNLASGIPDRFSGSGSGTDFTLTISRLEPEDFAVYYCQQWSSYPFTFGQGTKVEIK

SEQ ID NO: 29 - protein, 450 aa, Synthetic construct
QVQMVQSGAEVKKPGASVKVSCKASGFNIKDTYIHWVRQAPGQGLEWMGEIDPANDNTKYDPKFQGRVTITADTSTSTVYMELSSLRSEDTAVYYCATYYYKYDVGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 215 aa, Synthetic construct
EIVLTQSPGTLSLSPGERATLSCSVSSSISSSNLHWYQQKPGQAPRLLIYGTSNLASGIPDRFSGSGSGTDFTLTISRLEPEDFAVYYCQQWSSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 32 - protein, 7 aa, Synthetic construct
AASNRGS

SEQ ID NO: 33 - protein, 119 aa, Synthetic construct
QVQLQQPGAELVRPGASVKLSCKASGYSFTSYWMNWVKQRPGQGLEWIGVIHPSDSETWLDQKFKDKATLTVDKSSTTAYMQLISPTSEDSAVYYCAREHYGTSPFAYWGQGTLVTVSS

SEQ ID NO: 34 - DNA, 357 bp, Synthetic construct
  source 1..357: mol_type = other DNA
gaggtccaactgcagcagcctggggctgaactggtgaggcctggagcttcagtgaagctgtcctgcaaggcttctggctactccttcaccagctactggatgaactgggtgaagcagaggcctggacaaggccttgagtggattggcgtgattcatccttccgatagtgaaacttggttagatcagaagttcaaggacaaggccacattgactgtagacaaatcctccaccacagcctacatgcaactcatcagcccgacatctgaggactctgcggtctattactgtgcaagggagcactacggtactagcccgtttgcttactggggccaagggactctggtcactgtgtcttcc

SEQ ID NO: 35 - DNA, 357 bp, Synthetic construct
  source 1..357: mol_type = other DNA
caagttcaattggtacagagcggggcagaggtgaagaaacccggcgccagtgttaaggtgtcctgcaaagccagcggttacagctttacaagctattggatgaattgggtgcgtcaagcaccagggcagggtctggaatggattggggtgatacatccttctgacagcgaaacatggttggaccagaaatttaaagatcgtgtgacaattacagtcgataagtccacaagcactgcttacatggaactctccagcttgcggtccgaggacaccgctgtgtattattgcgccagagagcactacggcacatcaccttttgcatactggggccagggaactctcgtaaccgtatcctcc

SEQ ID NO: 36 - protein, 119 aa, Synthetic construct
QVQLVQSGAEVKKPGASVKVSCKASGYSFTSYWMNWVRQAPGQGLEWAGVIHPSDSETWLDQKFKDRVTITVDKSTSTAYMELSSLRSEDTAVYYCAREHYGTSPFAYWGQGTLVTVSS

SEQ ID NO: 37 - DNA, 357 bp, Synthetic construct
  source 1..357: mol_type = other DNA
caagttcaattggtacagagcggggcagaggtgaagaaacccggcgccagtgttaaggtgtcctgcaaagccagcggttacagctttacaagctattggatgaattgggtgcgtcaagcaccagggcagggtctggaatgggctggggtgatacatccttctgacagcgaaacatggttggaccagaaatttaaagatcgtgtgacaattacagtcgataagtccacaagcactgcttacatggaactctccagcttgcggtccgaggacaccgctgtgtattattgcgccagagagcactacggcacatcaccttttgcatactggggccagggaactctcgtaaccgtatcctcc

SEQ ID NO: 38 - protein, 111 aa, Synthetic construct
DIVLTQSPASLAVSLGQRATISCRANESVDNYGMSFMNWFQQKPGQPPKLLIHAASNQGSGVPARFSGSGFGTDFSLNIHPMEEDDAAMYFCQQSKEVPYTFGGGTKLEIK

SEQ ID NO: 39 - DNA, 333 bp, Synthetic construct
  source 1..333: mol_type = other DNA
gacattgtgctgacccaatctccagcttctttggctgtgtctctagggcagagggccaccatctcctgcagagccaacgaaagtgttgataattatggcatgagttttatgaactggttccaacagaaaccaggacagccacccaaactcctcatccatgctgcatccaaccaaggatccggggtccctgccaggtttagtggcagtgggtttgggacagacttcagcctcaacatccatcctatggaggaggatgatgctgcaatgtatttctgtcagcaaagtaaggaggttccgtacacgttcggaggggggaccaagctggaaataaaa

SEQ ID NO: 40 - DNA, 333 bp, Synthetic construct
  source 1..333: mol_type = other DNA
gaaatcgtactcacccagtcacctgcaaccctttctctgagccccggtgaacgtgccactctcagctgcagagcaagtgagagtgtggacaattacggcatgtccttcatgaactggtttcagcagaagcctgggcagccacctaagctgctcatccacgccgcctctaaccagggatctggggtgccttcacgtttttctggatcaggaagtggcactgacttcacccttacaatcagctctctggagccagaggactttgccgtctatttctgccagcaatctaaagaggtgccctatacttttggtggcgggaccaaggttgagatcaaa

SEQ ID NO: 41 - protein, 111 aa, Synthetic construct
EIVLTQSPATLSLSPGERATLSCRANESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 42 - DNA, 333 bp, Synthetic construct
  source 1..333: mol_type = other DNA
gaaatcgtactcacccagtcacctgcaaccctttctctgagccccggtgaacgtgccactctcagctgcagagcaaatgagagtgtggacaattacggcatgtccttcatgaactggtttcagcagaagcctgggcagccacctaagctgctcatccacgccgcctctaaccagggatctggggtgccttcacgtttttctggatcaggaagtggcactgacttcacccttacaatcagctctctggagccagaggactttgccgtctatttctgccagcaatctaaagaggtgccctatacttttggtggcgggaccaaggttgagatcaaa

SEQ ID NO: 43 - protein, 111 aa, Synthetic construct
EIVLTQSPATLSLSPGERATLSCRASESVDNYGMSFMNWFQQKPGQPPKLLIHAASNRGSGVPSRFSGSGSGTDFTLTISSLEPEDFAVYFCQQSKEVPYTFGGGTKVEIK

SEQ ID NO: 44 - DNA, 333 bp, Synthetic construct
  source 1..333: mol_type = other DNA
gaaatcgtactcacccagtcacctgcaaccctttctctgagccccggtgaacgtgccactctcagctgcagagcaagtgagagtgtggacaattacggcatgtccttcatgaactggtttcagcagaagcctgggcagccacctaagctgctcatccacgccgcctctaaccgcggatctggggtgccttcacgtttttctggatcaggaagtggcactgacttcacccttacaatcagctctctggagccagaggactttgccgtctatttctgccagcaatctaaagaggtgccctatacttttggtggcgggaccaaggttgagatcaaa

SEQ ID NO: 45 - protein, 280 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 46 - protein, 6 aa, Synthetic construct
SSYAMS

SEQ ID NO: 47 - protein, 12 aa, Synthetic construct
  VARIANT 1: note = X is R or G
XASQSVSSSYLA

SEQ ID NO: 48 - protein, 11 aa, Synthetic construct
  VARIANT 1: note = X is R or G
XASQSVSSYLA

SEQ ID NO: 49 - protein, 12 aa, Synthetic construct
RASQSVSSSYLA

SEQ ID NO: 50 - protein, 7 aa, Synthetic construct
  VARIANT 1: note = X is D or G
  VARIANT 4: note = X is N, S, or T
XASXRAT

SEQ ID NO: 51 - protein, 7 aa, Synthetic construct
DASSRAT

SEQ ID NO: 52 - protein, 9 aa, Synthetic construct
  VARIANT 8: note = J is L or I
QQYGSSPJT

SEQ ID NO: 53 - protein, 9 aa, Synthetic construct
QQYGSSPIT

SEQ ID NO: 54 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 55 - protein, 12 aa, Synthetic construct
  VARIANT 4: note = X is K or R
  VARIANT 6: note = X is D or E
YYYXYXVGGFDY